(19) United States
(12) Patent Application Publication
(10) Pub. No.: US 2012/0035244 A1
(43) Pub. Date: Feb. 9, 2012

(54) PARP1 TARGETED THERAPY

(75) Inventors: Arul M. Chinnaiyan, Plymouth, MI (US); J. Chad Brenner, Ann Arbor, MI (US); Anastasia Yocum, Ann Arbor, MI (US)

(73) Assignee: THE REGENTS OF THE UNIVERSITY OF MICHIGAN, ANN ARBOR, MI (US)

(21) Appl. No.: 13/192,159

(22) Filed: Jul. 27, 2011

Related U.S. Application Data
(60) Provisional application No. 61/368,810, filed on Jul. 29, 2010.

(51) Int. Cl. A61K 31/713; A61P 35/00; A61K 31/502; C12N 5/09; C12N 9/99

(52) U.S. Cl. 514/44 A; 435/375; 435/184; 514/248

(57) ABSTRACT

The present invention relates to compositions and methods for cancer therapy, including but not limited to, targeted inhibition of cancer markers. In particular, the present invention relates to PARP1 proteins and nucleic acids as clinical and research targets for cancer.

This application claims priority to provisional application 61/368,810, filed Jul. 29, 2010, which is herein incorporated by reference in its entirety.

### STATEMENT REGARDING FEDERALLY SPONSORED RESEARCH OR DEVELOPMENT

This invention was made with government support under CA069568, CA132874 and CA111275 awarded by the National Institutes of Health and W81XWH-06-1-0224 awarded by the Army Medical Research and Material Command. The government has certain rights in the invention.

### FIELD OF THE INVENTION

The present invention relates to compositions and methods for cancer therapy, including but not limited to, targeted inhibition of cancer markers. In particular, the present invention relates to PARP1 proteins and nucleic acids as clinical and research targets for cancer.

### BACKGROUND OF THE INVENTION

A central aim in cancer research is to identify altered genes that are causally implicated in oncogenesis. Several types of somatic mutations have been identified including base substitutions, insertions, deletions, translocations, and chromosomal gains and losses, all of which result in altered activity of an oncogene or tumor suppressor gene. First hypothesized in the early 1900's, there is now compelling evidence for a causal role for chromosomal rearrangements in cancer (Rowley, Nat Rev Cancer 1: 245 (2001)). Recurrent chromosomal aberrations were thought to be primarily characteristic of leukemias, lymphomas, and sarcomas. Epithelial tumors (carcinomas), which are much more common and contribute to a relatively large fraction of the morbidity and mortality associated with human cancer, comprise less than 1% of the known, disease-specific chromosomal rearrangements (Mitelman, Mutat Res 462: 247 (2000)). While hematological malignancies are often characterized by balanced, disease-specific chromosomal rearrangements, most solid tumors have a plethora of non-specific chromosomal aberrations. It is thought that the karyotypic complexity of solid tumors is due to secondary alterations acquired through cancer evolution or progression.

Two primary mechanisms of chromosomal rearrangements have been described. In one mechanism, promoter/enhancer elements of one gene are rearranged adjacent to a proto-oncogene, thus causing altered expression of an oncogenic protein. This type of translocation is exemplified by the apposition of immunoglobulin (IG) and T-cell receptor (TCR) genes to MYC leading to activation of this oncogene in B- and T-cell malignancies, respectively (Rabbitts, Nature 372: 143 (1994)). In the second mechanism, rearrangement results in the fusion of two genes, which produces a fusion protein that may have a new function or altered activity. The prototypic example of this translocation is the BCR-ABL gene fusion in chronic myelogenous leukemia (CML) (Rowley, Nature 243: 290 (1973); de Klein et al., Nature 300: 765 (1982)). Importantly, this finding led to the rational development of imatinib mesylate (Gleevec), which successfully targets the BCR-ABL kinase (Deininger et al., Blood 105: 2640 (2005)). Thus, therapies that specifically target common epithelial tumors are needed.

### SUMMARY OF THE INVENTION

The present invention relates to compositions and methods for cancer therapy, including but not limited to, targeted inhibition of cancer markers. In particular, the present invention relates to PARP1 proteins and nucleic acids as clinical and research targets for cancer.

Embodiments of the present invention provide compositions and methods for determining if a subject's cancer harbors ETS gene fusions and determining a treatment course of action based on the presence of absence of the gene fusions. In some embodiments, in subjects having ETS gene fusions, PARP1 inhibitors find use as anti-cancer therapeutic agents.

For example, in some embodiments, the present invention provides a method of inhibiting a biological activity of PARP1 in a cell, wherein the cell comprises a gene fusion of an ETS family member gene, comprising contacting the cell with a molecule that inhibits at least one biological activity (e.g., promoting growth or invasion of the cell) of PARP1. In some embodiments, the ETS family member gene is ERG or ETV1. In some embodiments, the ETS family member gene is fused to an androgen regulated gene. In some embodiments, the ETS family member gene is ERG and the androgen regulated gene is TMPRSS2. In some embodiments, the molecule is an siRNA or a small molecule (e.g., including, but not limited to, 8-hydroxy-2-methylquinazolinone (NU1025), AZD2281 (Olaparib), BSI-201, ABT-888, AG014699, CEP 9722, MK 4827, LT-673 or 3-aminobenzamide). In some embodiments, the cell is a cancer cell (e.g., a prostate cancer cell, a Ewing's sarcoma cell or an T-cell acute lymphoblastic leukemia cell). In some embodiments, the cell is in vivo (e.g., in an animal such as a human or a non-human mammal). In some embodiments, the cell is ex vivo. In some embodiments, the cell has an increased level of DNA damage (e.g., double stranded breaks) relative to a control cell (e.g., a cancer cell that does not have an ETS family member fusion or a non-cancerous cell).

Further embodiments of the present invention provide a method, comprising: a) assaying a cancer sample from a subject (e.g., a subject diagnosed with prostate cancer) for the presence or absence of a gene fusion comprising an ETS family member; and b) administering a PARP1 inhibitor to the subject when the cancer sample has the presence of the gene fusion comprising an ETS family member. In some embodiments, the PARP1 inhibitor inhibits at least one biological activity of PARP1 (e.g., growth of the cell or invasion of the cell). In some embodiments, subjects are tested for the presence or absence of ETS family member gene fusions prior to treatment with a PARP1 inhibitor or other anti-cancer therapeutic. In some embodiments, individuals are re-tested for the presence or absence of ETS family member gene fusions after treatment is administered. In some embodiments, subjects are treated with a PARP1 inhibitor or other anti-cancer agent prior to testing for the presence or absence of an ETS family member gene fusion. In some embodiments, treatment is altered based on the results of the assay for an ETS family member gene fusion. In some embodiments, a second agent (e.g., a known chemotherapeutic agent such as, for example, temozolomide) is administered in combination with the PARP1 inhibitor.

Additional embodiments are described herein.

### DESCRIPTION OF THE FIGURES

FIG. 1 shows that the TMPRSS-ERG gene fusion product interacts with PARP1 and the DNA-PK complex. a, Mass spectrometric analysis of proteins interacting with ERG. Histograms show peptide coverage of ERG, DNA-PKcs, Ku70 and Ku80. b, ERG, DNA-PKcs, PARP1 but not Ku70 or Ku80, interact independent of DNA. c, ERG, DNA-PKcs, PARP1, Ku70 and Ku80 associate in ERG gene fusion positive human prostate cancer tissues. d, Schematic of TMPRSS2-ERG gene fusion expression vectors. e, Immunoprecipitation of DNA-PKcs, Ku70, Ku80 and PARP1 from HEK293 cells transfected with ERG expression vectors depicted in d. f. Schematic representation of halo-tagged ERG fragment vectors. g. Arrow indicates Y373, the amino acid required for the ERG:DNA-PKcs interaction.

FIG. 2 shows that PARP1 and DNA-PKcs are required for ERG-regulated transcription. a, Chromatin immunoprecipitation (ChIP) of PARP1 and the DNAPK complex shows an association with ERG-regulated targets including the PLA1A promoter as well as FKBP5, PSA and TMPRSS2 enhancers, but not the negative control gene KIAA0066. b, PLA1A promoter activity as assessed by relative luminescence following transduction with LACZ or ERG adenovirus and siRNA as indicated in RWPE cells. c, Gene expression arrays were performed using RNA from VCaP cells treated with either PARP1 or DNA-PKcs siRNA. d, qPCR data from VCaP cells treated with siRNA of indicated. e, VCaP cells were treated with either NU7026 or Olaparib for 48 hours as indicated and qPCR analysis of ERG-target genes was performed.

FIG. 3 shows that ERG-mediated invasion requires engagement of PARP1 and DNA-PKcs. a, RWPE cells were infected with ERG and indicated siRNAs or different doses of the DNA-PKcs inhibitor NU7026 or the small molecule PARP1 inhibitor NU1025 and cell invasion quantified. b, As in a, except RWPE-ETV1 cells. c, As in a, except VCaP cells. d, as in a, except PC3 or RWPE-SLC45A3-BRAF cells. e, Chiecken chorioallantoic membrane (CAM) intravasation assay performed using stable RWPE-ERG cells and siRNA or 40 mg/kg Olaparib. f, Liver metastasis in chicken embryos was assessed 8 days following implantation of cells into the upper CAM. Eggs were treated with Olaparib as in e as indicated (for all experiments mean+/−SEM shown) * P<0.05, ** P<0.01. g. ETS-positive (VCaP and LNCaP) and ETS-negative (PC3 and 22RV1) prostate cancer cells as well as BRCA1 mutant (HCC1937) and BRCA1/2 WT (MDAMB-231) breast cancer cells were implanted onto the upper CAM.

FIG. 4 shows that inhibition of PARP1 attentuates ETS positive, but not ETS negative cell line xenograft growth. a, VCaP (ERG+) and, b, PC3 (ETS−) cell line xenografted mice were treated with Olaparib or NU7026 as indicated. c, DU145 (ETS−) and, d, 22RV1 (ETS−) xenografted mice were treated with Olaparib as indicated. Caliper measurements were taken weekly. * P<0.01.

FIG. 5 shows that PARP1 inhibition selectively attentuates ETS positive xenograft and primagraft growth. a and b, Overexpression of ERG in PC3 cells (ETS−) confers sensitivity PARP inhibition. PC3-LACZ and PC3-ERG cells were used as isogenic controls to assess specific effects of Olaparib or NU7026 as indicated. c, Mice with ERG positive primagrafts (MDA-PCa-133) were treated as indicated with or without 40 mg/kg Olaparib. d, ETV1 positive (MDA-PCa-2b-T) and, e, ETS negative (MDA-PCa-118b) primagrafts used as in c. * P<0.01. f. Mice xenografted with VCaP cells were treated as in (A) except with 100 mg/kg Olaparib and/or 50 mg/kg TMZ as indicated

FIG. 6 shows that ETS transcription factors induce DNA damage which is potentiated by PARP inhibition. a, γ-H2A.X immunofluorescence staining shows that ERG induces the formation of γ-H2A.X foci. b, Quantification of γ-H2A.X and 53BP1 immunofluorescence staining in PrEC or VCaP cells. c, ETS overexpression or BRCA2 knockdown (with shRNA) induces DNA damage as assessed by neutral COMET assay in VCaP cells. d, Quantification of average COMET tail moments following treatment as noted in the box plot. e, Exemplary model to therapeutically target ETS gene fusions via their interacting enzyme, PARP1 and interacting kinase DNA-PKcs.

FIG. 7 shows a schematic representation of mass spectrometry workflow used for identification of ERG interacting proteins.

FIG. 8 shows that ERG interacts with DNAPK-cs, Ku70 and Ku80. a, Co-immunoprecipitation of V5-ERG, DNA-PKcs, Ku70 and Ku80 confirmed by immunoblot. VCaP cells were infected with adenovirus encoding V5-ERG. b, Untreated VCaP cells were used to show that the endogenous gene fusion product interacts with DNA-PKcs, Ku70 and Ku80, but not ATR. C. IP-Western blot analysis of RWPE cells (low endogenous ERG) transduced with ERG-V5 adenovirus 48 hours prior to harvesting total cell lysate. Immunoprecipitations were performed with different antibodies as indicated.

FIG. 9 shows a. that androgen receptor interacts with DNA-PKcs, Ku70, Ku80 and PARP1 in VCaP cells. b. IP-Western performed on VCaP cells using agarose coupled-PARP1 or IgG antibodies. c. Human prostate cancer tissue samples were collected from the UM warm autopsy program. d. HEK293 cells were transfected with full length wild type ERG and used for IP-Western blot analysis. e. Three tissues with ERG rearrangement were used to perform IP-Western blot analysis using an agarose coupled ERG antibody in the presence or absence of ethidium bromide as indicated. Membranes were blot for PARP1 and ERG expression.

FIG. 10 shows that domain and sequence mapping of the DNA-PKcs and PARP1-ETS interaction. a, Absolute complexity of the three additional ETS gene paralogues selected for immunoprecipitation experiments. Outside of the conserved pointed and ETS DNA binding domains, this plot shows low amino acid sequence homology with ERG. b, c and d, Immunoprecipitation-Western blot analysis of HEK293 cells transfected with either an FLAG-ETS1, FLAG-SPI1 or FLAG-ETV1 expression vector, respectively. Experiments were completed three times. e. IP-Western performed using purified halo-tagged ERG, ETS1, ETV1 and SPI1 as well as purified DNA-PKcs. f. and g IP-Western blots performed using HALO ligand linked bead to map DNA-PKcs:ERG and PARP1:ERG interactions, respectively. h. IP-Western using purified HALO-ETS constructs with different alanine mutations as indicated and purified DNA-PKcs.

FIG. 11 shows that the DNA-PKcs:PARP1 complex associates with ERG regulated genomic loci in VCaP cells. a, Chromatin immunoprecipitation assays performed on untreated VCaP cells. b, VCaP cells were treated with either scrambled control or ERG siRNA and mRNA expression changes were analyzed by qRT-PCR. Experiment was run in triplicate. c. Co-recruitment as assessed by re-ChIP assays. d. IP-Western blot analysis of a DNA-PKcs pulldown from VCaP cells. Representative images are shown. e. RWPE cells transduced with either LACZ or ERG adenovirus were treated with siRNA for 48 hours prior to analysis for ETS target gene expression (PLA1A) by qPCR or promoter activity by luminescence assay. f and g, Analysis of genes greater than 2-fold up- or down-regulated, respectively, following siRNA treatments as indicated.

FIG. 12 shows validation of siRNA knockdown in RWPE cell line model. a, qPCR analysis of RNA isolated in parallel to the invasion and promoter reporter assays forty eight hours after adenoviral transduction and siRNA treatment.

FIG. 13 shows validation of siRNA knockdown in VCaP cell line model. a, qPCR analysis of RNA isolated in parallel to the invasion assay.

FIG. 14 shows Venn diagram analysis of gene expression array data. a and b, Analysis of genes greater than 2-fold up- or down-regulated, respectively, following siRNA treatments as indicated. c, RWPE cells transduced with either LACZ or ETV1 lentivirus (as in FIG. 3c) were treated with siRNA for 48 hours prior to analysis for ETS target gene expression by qPCR.

FIG. 15 shows that Olaparib blocks ERG-mediated RWPE cell invasion. a, qRT-PCR analysis of cell treated with siRNA or 25 mM Olaparib as indicated. Additional DNA-PKcs, ATM and XRCC4 siRNAs were analyzed. b, Matrigel-coated Boyden chamber cell invasion assays were quantified. Representative images are shown for each treatment condition. Experiment was run three times in triplicate (with mean+/−SEM shown). * p<0.05, ** p<0.01.

FIG. 16 shows that Olaparib blocks VCaP cell invasion. a, qRT-PCR analysis of cell treated with siRNA or 25 mM Olaparib as indicated. b, Matrigel-coated Boyden chamber cell invasion assays were quantified.

FIG. 17 shows qPCR confirmation of knockdown in RWPE-ETV1, PC3 and RWPE_SLC45A3-BRAF cell models and that Olaparib does now effect invasion in these models. a, qRT-PCR analysis of RWPE-ETV1 cells treated with siRNA, 100 mM NU7026 or 25 mM Olaparib as indicated. b, As in a except with PC3, and RWPE_SLC45A3-BRAF models.

FIG. 18 shows a. Panel of cell lines treated with Olaparib. b. Olaparib concentrations were extended for several cell lines in order to determine the short term (72 hour) EC50 value.

c. NU7026 chemosensitivity assay.

FIG. 19 shows that PARP1 expression and activity is required for ERG-mediated intravasation in vivo. a, qPCR analysis of gene expression changes following siRNA treatment of cells at the time of implantation into the upper CAM and at the time of harvest from the CAM. b, Tumor weight was measured from xenografted cell lines treated with Olaparib for 10 days.

FIG. 20 shows establishment of PC3-ERG model cell line. a, Western blot analysis of LACZ and ERG overexpression in PC3 model. b, Immunoprecipitation-Western blot analysis confirms that ERG interacts with DNA-PKcs, PARP1, Ku70 and Ku80 in PC3 cells. c, Chromatin immunoprecipitation assay using anti-ERG antibody. d, qPCR validation of ERG overexpression and increased expression of the ERG target gene PLA1A confirms functional ERG expression.

FIG. 21 shows establishment of in vivo effects of Olaparib and characterization of primagraft model. a, Western blot analysis to assess total PAR levels of ERG overexpressing PC3 cells treated with or without 40 mg/kg Olaparib 4 hours prior to harvesting tumor. b, RNA harvested from 4 hour staged tumors as in a or with 25 mg/kg NU7026 was analyzed by qPCR. c and d, as in b, analyzing for total ERG mRNA expression or several ERG target genes as indicated, respectively. e, qPCR assessment of ETS gene expression in the primagraft models and control cell lines. f, qPCR was performed for several ETS-regulated target genes as in e.

FIG. 22 shows that Olaparib does not reduce total mouse body weight. a, Mice from VCaP cell xenograft experiments were weighed at the time of caliper measurement. b, c and d, As with a, except PC3-Control, PC3-LACZ and PC3-ERG models, respectively. e, f, g, h, i, As with a, except DU145, 22RV1 xenografts and MDA-PCa-133, MDA-PCa-2b-T as well as MDA-PCa-118b primagrafts. j, Serum from PC3-ERG mice treated as indicated were assessed for alanine aminotransferase (ALT) and aspartate aminotransferase (AST) to determine liver toxicity.

FIG. 23 shows characterization of xenograft tumors reveals no significant change in proliferation or microvessel density. a, Representative photomicrographs of Ki67 immunohistochemical (IHC) staining in PC3-ERG xenografts treated with or without Olaparib. b, Quantification of Ki67 staining from VCaP and PC3 xenograft experiments. c, Representative images of CD31 IHC staining from PC3-ERG xenografts treated as indicated. d, Quantification of microvessel density as assessed by CD31 positivity.

FIG. 24 shows QRT-PCR confirmation of expression changes in PrEC and VCaP cells. PrEC cells treated with LACZ, EZH2, ETV1 or ERG lentivirus or VCaP cells treated with siRNA for 48 hours and used for g-H2A.X imaging were assessed for changes in the total RNA expression level of a, EZH2, b, ETV1 or, c and d, ERG as compared to GAPDH control.

FIG. 25 shows ETS gene fusion proteins induce g-H2A.X and 53BP1 foci formation. a, RWPE cells transduced with lentivirus overexpressing ERG, ETV1 or ETV5 were analyzed by immunofluorescence. b, Various prostate cell lines were assessed for percentage of cells displaying g-H2A.X foci following lentiviral infection as indicated. Expression changes were confirmed by qPCR (Data not shown). c, VCaP cells treated with siRNA or PC3 cell transduced with lentivirus as indicated were treated with or without Olaparib for 48 hours. d, PC3 cells transduced with lentivirus expressing BRCA2 shRNAs were analyzed for changes in BRCA2 mRNA expression as compared to GAPDH.

FIG. 26 shows a. Cells were treated with Olaparib for indicated times and harvested for COMET assays. b. Targeted DNA damage qRT-PCR arrays were run with PC3-LACZ versus PC3-ERG overexpressing cells. c. COMET assays run on PC3-LACZ, PC3-ERG or HCC1937 (BRCA1 mutant) cells treated with siRNA for 48 hours as indicated. d. qPCR quantification of knockdown efficiency of cells treated as in c. e. Homologous recombination efficiency assays were run by transfecting reporter constructs into stable cell lines.

FIG. 27 shows that Ewing's sarcoma cell lines are sensitive to PARP inhibition. a. CADO-ES1 or RD-ES1 cells were treated with siRNA for 48 hours. b. Soft agar colony formation was performed on cells treated with or without Olaparib as indicated. c. Neutral COMET assays were performed on a panel of Ewing's Sarcoma cell lines. d. As in C, except cells were pre-treated with siRNA for 48 hours. All experiments were run in triplicate. Error bars are standard error of the mean.

FIG. 28 shows that T-ALL cell lines are sensitive to PARP inhibition. a. Western blot analysis of several T-ALL cell lines comparing total ERG expression levels. b. Neutral COMET assays were performed on a panel of T-ALL cell lines. c. Soft agar colony formation was performed on cells treated with or without Olaparib as indicated.

FIG. 29 shows that ETS overexpression causes radioresistance, which is reversed by PARP inhibition. a. Isogenic PC3 or DU145 models overexpressing either T2:ERG or ERG with a deleted ETS domain were pre-treated with or without 10 μM Olaparib and then treated with different doses of radiation as indicated. Colony formation was assessed. b. As in A, except cells were pre-treated with either PARP1 or control siRNA prior to radiation. Western blot analysis used to assess PARP knockdown efficiency in the inset. c. Total Poly(ADP-ribose) (or PAR) levels were assessed by Western blot analysis from total cell lysates.

FIG. 30 shows that ETS overexpression causes faster repair of DNA double strand breaks, which is reversed by PARP inhibition. a. and b. Isogenic PC3 and DU145 models, respectively, overexpressing either T2:ERG or ERG with a deleted ETS domain were pre-treated with or without 10 μM Olaparib and then treated with radiation as indicated. Immunofluorescence staining of γH2A.X foci was used to assess total levels of DNA damage and repair rate.

### DEFINITIONS

To facilitate an understanding of the present invention, a number of terms and phrases are defined below:

As used herein, the term “inhibits at least one biological activity of PARP1” refers to any agent that decreases any activity of PARP1 (e.g., including, but not limited to, the activities described herein), via directly contacting PARP1 protein, contacting PARP1 mRNA or genomic DNA, causing conformational changes of PARP1 polypeptides, decreasing PARP1 protein levels, or interfering with PARP1 interactions with signaling partners, and affecting the expression of PARP1 target genes. Inhibitors also include molecules that indirectly regulate PARP1 biological activity by intercepting upstream signaling molecules.

As used herein, the term “gene fusion” refers to a chimeric genomic DNA, a chimeric messenger RNA, a truncated protein or a chimeric protein resulting from the fusion of at least a portion of a first gene to at least a portion of a second gene. The gene fusion need not include entire genes or exons of genes.

As used herein, the terms “detect”, “detecting” or “detection” may describe either the general act of discovering or discerning or the specific observation of a detectably labeled composition.

As used herein, the term “subject” refers to organisms to be treated by the methods of the present invention. Such organisms preferably include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of a PARP1 inhibitor and optionally one or more other agents) for prostate cancer.

The term “diagnosed,” as used herein, refers to the recognition of a disease by its signs and symptoms, or genetic analysis, pathological analysis, histological analysis, and the like.

A “subject suspected of having cancer” encompasses an individual who has received an initial diagnosis (e.g., a CT scan showing a mass or increased PSA level) but for whom the stage of cancer or presence or absence of ETS family member gene fusions is not known. The term further includes people who once had cancer (e.g., an individual in remission). In some embodiments, “subjects” are control subjects that are suspected of having cancer or diagnosed with cancer.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the ETS family gene fusions disclosed herein.

As used herein, the term “characterizing prostate tissue in a subject” refers to the identification of one or more properties of a prostate tissue sample (e.g., including but not limited to, the presence of cancerous tissue, the presence or absence of ETS family member gene fusions, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “effective amount” refers to the amount of a compound (e.g., a PARP1 inhibitor) sufficient to effect beneficial or desired results. An effective amount can be administered in one or more administrations, applications or dosages and is not limited intended to be limited to a particular formulation or administration route.

As used herein, the term “co-administration” refers to the administration of at least two agent(s) (e.g., a PARP1 inhibitor) or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “toxic” refers to any detrimental or harmful effects on a cell or tissue as compared to the same cell or tissue prior to the administration of the toxicant.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION OF THE INVENTION

The present invention relates to compositions and methods for cancer therapy, including but not limited to, targeted inhibition of cancer markers. In particular, the present invention relates to PARP1 proteins and nucleic acids as clinical and research targets for cancer.

ETS transcription factors are aberrantly expressed in a diverse array of cancers including prostate, breast, melanoma and Ewing's sarcoma (Jeon et al. Oncogene 10, 1229-1234 (1995); Sorensen et al. Nature genetics 6, 146-151 (1994); Tognon et al. Cancer cell 2, 367-376 (2002); Shurtleff et al. Leukemia 9, 1985-1989 (1995); Tomlins et al. Science (New York, N.Y. 310, 644-648 (2005); Jane-Valbuena et al. Cancer research 70, 2075-2084). In prostate cancer, recurrent gene fusions of the androgen-regulated gene, TMPRSS2, to the oncogenic ETS transcription factor ERG are present in greater than 50% of prostate cancers (Tomlins et al., 2005; supra; Kumar-Sinha et al. Nature reviews 8, 497-511 (2008); Brenner and Chinnaiyan, Biochimica et biophysica acta (2009)). Although ERG is the predominant ETS gene rearrangement observed, other ETS transcription factors are found at a much lower frequency in prostate cancer, including ETV1, ETV4 and ETV5 (Helgeson et al. prostate cancer. Cancer research 68, 73-80 (2008)). ETS gene fusions appear early in prostatic disease during the transition from high-grade prostatic intraepithelial neoplasia (PIN) lesions to invasive carcinoma (Helgeson et al. prostate cancer. Cancer research 68, 73-80 (2008); Tomlins et al. Nature 448, 595-599 (2007); Tomlins, S. A., et al. Neoplasia (New York, N.Y. 10, 177-188 (2008); Klezovitch et al. Proceedings of the National Academy of Sciences of the United States of America 105, 2105-2110 (2008); Wang et al. Cancer research 68, 8516-8524 (2008); Hermans et al. Cancer research 68, 7541-7549 (2008)) and are formed by several mechanisms including interstitial deletion and genomic insertion (Perner et al. The American journal of surgical pathology 31, 882-888 (2007)). In prostate cell lines devoid of the TMPRSS2:ERG gene fusion, it has been shown that androgen receptor-induced proximity combined with ionizing radiation triggers spontaneous TMPRSS2:ERG gene fusion formation (Lin et al. Cell 139, 1069-1083 (2009); Mani, et al. Science (New York, N.Y. 326, 1230 (2009)).

Once an ETS gene fusion is formed through genomic rearrangement, the subsequent overexpression of an ETS gene fusion protein can contribute to cancer progression through several different mechanisms. For example, TMPRSS2-ERG gene fusion expression is required for cell growth in cell line models that harbor an endogenous gene fusion both in vitro and in vivo (Tomlins et al., 2007 Nature 448, 595-599 (2007); Tomlins et al., 2008; supra; Wang et al., 2008; supra; Sun, et al. Oncogene 27, 5348-5353 (2008)). Likewise, ETS proteins are active transcription factors that drive cellular invasion through the induction of a transcriptional program highly enriched for invasion-associated genes (Helgeson et al., supra; Tomlins et al. 2007 Nature 448, 595-599 (2007); Tomlins et al., 2008; supra; Klezovitch et al., supra; Want et al., 2008; supra; Hermens et al., supra). Genetically-engineered mice expressing ERG or ETV1 under androgen regulation exhibit PIN-like lesions, but do not develop frank carcinoma, indicating that additional collaborating mutations may be required for de novo carcinogenesis (Tomlins et al. 2007 Nature 448, 595-599 (2007); Tomlins et al., 2008; supra; Klezovitch et al., supra; Carver, B. S., et al. Aberrant ERG expression cooperates with loss of PTEN to promote cancer progression in the prostate. Nature genetics 41, 619-624 (2009); King et al. Nature genetics 41, 524-526 (2009); Zong et al. Proceedings of the National Academy of Sciences of the United States of America (2009)). Overexpression of ERG leads to accelerated carcinogenesis in mouse prostates with deletion of the tumor suppressor PTEN (Carver al., supra; King et al., supra). Additionally, in a transplant model, mouse prostate epithelial cells that are forced to overexpress both ERG and the androgen receptor gene form invasive prostate cancer (Zong et al., supra). Experiments conducted during the course of development of the present invention demonstrated that ERG overexpression induces DNA damage and sensitizes cells to the action of PARP1 inhibitors. This indicates that ERG can function to accelerate prostate carcinogenesis.

### I. Diagnostic Methods

As described herein, embodiments of the present invention provide compositions and methods for inhibiting the activity of PARP1 proteins associated with cancer (e.g., prostate cancer, Ewing's sarcoma or T-cell acute lymphoblastic leukemia (T-ALL)). In some embodiments, PARP1 polypeptides or nucleic acids are targeted as anti-cancer therapeutics. In some embodiments, individuals are assayed for the presence of a gene fusion prior to, during, or following administering the anti-PARP1 therapeutic.

### A. Gene Fusions

In some embodiments, anti-PARP1 therapeutics are administered to subjects whose tumors exhibit ETS gene fusions. Exemplary ETS gene fusions are described below. In some embodiments, gene fusions are fusions between androgen regulated genes and ETS family member genes. Exemplary gene fusions are described, for example in U.S. Pat. No. 7,718,369, US patent publication 2009-0208937 and US patent publication 2009-0239221, each of which is herein incorporated by reference.

Genes regulated by androgenic hormones are of critical importance for the normal physiological function of the human prostate gland. They also contribute to the development and progression of prostate carcinoma. Recognized ARGs include, but are not limited to: DDX5; TMPRSS2; PSA; PSMA; KLK2; SNRK; Seladin-1; and, FKBP51 (Paoloni-Giacobino et al., Genomics 44: 309 (1997); Velasco et al., Endocrinology 145(8): 3913 (2004)). Transmembrane protease, serine 2 (TMPRSS2; NM_{—}005656), has been demonstrated to be highly expressed in prostate epithelium relative to other normal human tissues (Lin et al., Cancer Research 59: 4180 (1999)). The TMPRSS2 gene is located on chromosome 21. This gene is located at 41,750,797-41,801,948 bp from the pter (51,151 total bp; minus strand orientation). The human TMPRSS2 protein sequence may be found at GenBank accession no. AAC51784 (Swiss Protein accession no. O15393)) and the corresponding cDNA at GenBank accession no. U75329 (see also, Paoloni-Giacobino, et al., Genomics 44: 309 (1997)).

In some embodiments, gene fusions comprise transcriptional regulatory regions of an ARG. The transcriptional regulatory region of an ARG may contain coding or non-coding regions of the ARG, including the promoter region. The promoter region of the ARG may further contain an androgen response element (ARE) of the ARG. The promoter region for TMPRSS2, in particular, is provided by GenBank accession number AJ276404.

The E-twenty six (ETS) family of transcription factors regulate the intra-cellular signaling pathways controlling gene expression. As downstream effectors, they activate or repress specific target genes. As upstream effectors, they are responsible for the spacial and temporal expression of numerous growth factor receptors. Almost 30 members of this family have been identified and implicated in a wide range of physiological and pathological processes. These include, but are not limited to: ERG; ETV1 (ER81); FLI1; ETS1; ETS2; ELK1; ETV6 (TEL1); ETV7 (TEL2); GABPα; ELF1; ETV4 (E1AF; PEA3); ETV5 (ERM); ERF; PEA3/E1AF; PU.1; ESE1/ESX; SAP1 (ELK4); ETV3 (METS); EWS/FLI1; ESE1; ESE2 (ELF5); ESE3; PDEF; NET (ELKS; SAP2); NERF (ELF2); and FEV.

Ets Related Gene (ERG; NM_{—}004449), in particular, has been demonstrated to be highly expressed in prostate epithelium relative to other normal human tissues. The ERG gene is located on chromosome 21. The gene is located at 38,675,671-38,955,488 base pairs from the pter. The ERG gene is 279,817 total bp; minus strand orientation. The corresponding ERG cDNA and protein sequences are given at GenBank accession no. M17254 and GenBank accession no. NP04440 (Swiss Protein acc. no. P11308), respectively.

The ETS translocation variant 1 (ETV1) gene is located on chromosome 7 (GenBank accession nos. NC_{—}000007.11; NC_{—}086703.11; and NT_{—}007819.15). The gene is located at 13,708330-13,803,555 base pairs from the pter. The ETV1 gene is 95,225 bp total, minus strand orientation. The corresponding ETV1 cDNA and protein sequences are given at GenBank accession no. NM_{—}004956 and GenBank accession no. NP_{—}004947 (Swiss protein acc. no. P50549), respectively.

The human ETV4 gene is located on chromosome 14 (GenBank accession nos. NC_{—}000017.9; NT_{—}010783.14; and NT_{—}086880.1). The gene is at 38,960,740-38,979,228 base pairs from the pter. The ETV4 gene is 18,488 bp total, minus strand orientation. The corresponding ETV4 cDNA and protein sequences are given at GenBank accession no. NM_{—}001986 and GenBank accession no. NP_{—}01977 (Swiss protein acc. no. P43268), respectively.

### B. Diagnostic Methods

As described above, in some embodiments of the present invention, the gene fusion status of a subject's cancer (e.g., tumor) is determined prior to, during, or following administration of an anti-PARP1 therapeutic. Exemplary diagnostic methods are described below.

Any patient sample suspected of containing the gene fusions may be tested according to the methods of the present invention. By way of non-limiting examples, the sample may be tissue (e.g., a prostate biopsy sample or a tissue sample obtained by prostatectomy), blood, urine, semen, prostatic secretions or a fraction thereof (e.g., plasma, serum, urine supernatant, urine cell pellet or prostate cells). A urine sample is preferably collected immediately following an attentive digital rectal examination (DRE), which causes prostate cells from the prostate gland to shed into the urinary tract.

The patient sample typically requires preliminary processing designed to isolate or enrich the sample for the gene fusions or cells that contain the gene fusions. A variety of techniques known to those of ordinary skill in the art may be used for this purpose, including but not limited to: centrifugation; immunocapture; cell lysis; and, nucleic acid target capture (See, e.g., EP Pat. No. 1 409 727, herein incorporated by reference in its entirety).

In some embodiments, individuals are tested for the presence or absence of ETS family member gene fusions prior to, during or after treatment for prostate cancer. In some embodiments, treatment is determined or altered based on the presence or absence of ETS family member gene fusions (e.g., individuals with ETS family member gene fusions are administered PARP1 inhibitors). In some embodiments, treatment is altered after it has begun based on the presence or absence of ETS family member gene fusions. In some embodiments, a subject's cancer is assayed for ETS family member gene fusion status at intervals throughout treatment (e.g., to determine if treatment is effective) and treatment is altered (e.g., dosage, timing, therapeutic agent, etc.) as needed. In some embodiments, treatment is stopped based on the presence or absence of ETS family member gene fusions.

i. DNA and RNA Detection

The gene fusions of the present invention may be detected as chromosomal rearrangements of genomic DNA or chimeric mRNA using a variety of nucleic acid techniques known to those of ordinary skill in the art, including but not limited to: nucleic acid sequencing; nucleic acid hybridization; and, nucleic acid amplification.

1. Sequencing

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing, can be performed in a single reaction by labeling each of the di-deoxynucleotide chain-terminators with a separate fluorescent dye, which fluoresces at a different wavelength.

2. Hybridization

Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

In some embodiments, fusion sequences are detected using fluorescence in situ hybridization (FISH). The preferred FISH assays for the present invention utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor. Exemplary BAC probes can be found, for example in U.S. Pat. No. 7,718,369, herein incorporated by reference in its entirety.

The present invention further provides a method of performing a FISH assay on human prostate cells, human prostate tissue or on the fluid surrounding said human prostate cells or human prostate tissue. Specific protocols are well known in the art and can be readily adapted for the present invention. Guidance regarding methodology may be obtained from many references including: In situ Hybridization: Medical Applications (eds. G. R. Coulton and J. de Belleroche), Kluwer Academic Publishers, Boston (1992); In situ Hybridization: In Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo, et al., Am. J. Hum. Genet. 49:112-119 (1991); Klinger, et al., Am. J. Hum. Genet. 51:55-65 (1992); and Ward, et al., Am. J. Hum. Genet. 52:854-865 (1993)). There are also kits that are commercially available and that provide protocols for performing FISH assays (available from e.g., Oncor, Inc., Gaithersburg, Md.). Patents providing guidance on methodology include U.S. Pat. Nos. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

3. Microarrays

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

3. Amplification

Chromosomal rearrangements of genomic DNA and chimeric mRNA may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product. Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

4. Detection Methods

Non-amplified or amplified gene fusion nucleic acids can be detected by any conventional means. For example, the gene fusions can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

5. Detection of DNA Damage

In some embodiments, diagnostic methods detect DNA damage that may be indicative of ERG overexpression and an increased susceptibility of cancer cells to PARP1 inhibitors. In some embodiments, DNA double stranded breaks are detected using a histone marker of DNA double strand breaks called γ-H2A.X or the COMET assay (See e.g., Example 1 below).

ii. Protein Detection

The gene fusions of the present invention may be detected as truncated ETS family member proteins or chimeric proteins using a variety of protein techniques known to those of ordinary skill in the art, including but not limited to: protein sequencing; and, immunoassays.

1. Sequencing

Illustrative non-limiting examples of protein sequencing techniques include, but are not limited to, mass spectrometry and Edman degradation.

Mass spectrometry can, in principle, sequence any size protein but becomes computationally more difficult as size increases. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction, the peptide to be sequenced is adsorbed onto a solid surface (e.g., a glass fiber coated with polybrene). The Edman reagent, phenylisothiocyanate (PTC), is added to the adsorbed peptide, together with a mildly basic buffer solution of 12% trimethylamine, and reacts with the amine group of the N-terminal amino acid. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about 98%, which allows about 50 amino acids to be reliably determined.

2. Immunoassays

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex. A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups. An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

iii. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence or absence of a gene fusion) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

iv. In Vivo Imaging

Gene fusions may also be detected using in vivo imaging techniques, including but not limited to: radionuclide imaging; positron emission tomography (PET); computerized axial tomography, X-ray or magnetic resonance imaging method, fluorescence detection, and chemiluminescent detection. In some embodiments, in vivo imaging techniques are used to visualize the presence of or expression of cancer markers in an animal (e.g., a human or non-human mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of the present invention are useful in the identification of cancers that express gene fusions (e.g., prostate cancer). In vivo imaging is used to visualize the presence of a gene fusion. Such techniques allow for diagnosis without the use of an unpleasant biopsy. The in vivo imaging methods of the present invention can further be used to detect metastatic cancers in other parts of the body.

In some embodiments, reagents (e.g., antibodies) specific for the cancer markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin Onc 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl) EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the gene fusion, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

v. Compositions & Kits

Compositions for use in the diagnostic methods described herein include, but are not limited to, probes, amplification oligonucleotides, and antibodies. Particularly preferred compositions detect a product only when an ARG fuses to ETS family member gene. These compositions include, but are not limited to: a single labeled probe comprising a sequence that hybridizes to the junction at which a 5′ portion from a transcriptional regulatory region of an ARG fuses to a 3′ portion from an ETS family member gene (i.e., spans the gene fusion junction); a pair of amplification oligonucleotides wherein the first amplification oligonucleotide comprises a sequence that hybridizes to a transcriptional regulatory region of an ARG and the second amplification oligonucleotide comprises a sequence that hybridizes to an ETS family member gene; an antibody to an amino-terminally truncated ETS family member protein resulting from a fusion of a transcriptional regulatory region of an ARG to an ETS family member gene; or, an antibody to a chimeric protein having an amino-terminal portion from a transcriptional regulatory region of an ARG and a carboxy-terminal portion from an ETS family member gene.

Other useful compositions, however, include: a pair of labeled probes wherein the first labeled probe comprises a sequence that hybridizes to a transcriptional regulatory region of an ARG and the second labeled probe comprises a sequence that hybridizes to an ETS family member gene. Any of these compositions, alone or in combination with other compositions of the present invention, may be provided in the form of a kit. For example, the single labeled probe and pair of amplification oligonucleotides may be provided in a kit for the amplification and detection of gene fusions of the present invention. Kits may further comprise appropriate controls, detection reagents or analysis software.

The probe and antibody compositions of the present invention may also be provided in the form of an array.

### II. Therapeutic Applications

In some embodiments, the present invention provides therapies for cancer (e.g., prostate cancer, ALL or Ewing's sarcoma). In some embodiments, therapies directly or indirectly target PARP1 (e.g., in ETS gene fusion positive cancers).

A. RNA Interference and Antisense Therapies

In some embodiments, the present invention targets the expression of PARP1. For example, in some embodiments, the present invention employs compositions comprising oligomeric antisense or RNAi compounds, particularly oligonucleotides (e.g., those described herein), for use in modulating the function of nucleic acid molecules encoding gene fusions, ultimately modulating the amount of PARP1 expressed.

1. RNA Interference (RNAi)

In some embodiments, RNAi is utilized to inhibit PARP1 function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC(RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments. In some embodiments, RNAi oligonucleotides are designed to target the junction region of fusion proteins.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Comers, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7mers to 25mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol Biol. 2005 May 13; 348(4):883-93, J Mol Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

In some embodiments, the present invention utilizes siRNA including blunt ends (See e.g., US20080200420, herein incorporated by reference in its entirety), overhangs (See e.g., US20080269147A1, herein incorporated by reference in its entirety), locked nucleic acids (See e.g., WO2008/006369, WO2008/043753, and WO2008/051306, each of which is herein incorporated by reference in its entirety). In some embodiments, siRNAs are delivered via gene expression or using bacteria (See e.g., Xiang et al., Nature 24: 6 (2006) and WO06066048, each of which is herein incorporated by reference in its entirety).

In other embodiments, shRNA techniques (See e.g., 20080025958, herein incorporated by reference in its entirety) are utilized. A small hairpin RNA or short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. shRNA is transcribed by RNA polymerase III.

The present invention also includes pharmaceutical compositions and formulations that include the RNAi compounds of the present invention as described below.

2. Antisense

In other embodiments, PARP1 expression is modulated using antisense compounds that specifically hybridize with one or more nucleic acids encoding gene fusions. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of gene fusions. In the context of the present invention, “modulation” means either an increase (stimulation) or a decrease (inhibition) in the expression of a gene. For example, expression may be inhibited to prevent tumor proliferation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a gene fusion of the present invention. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in PCT Publ. No. WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy(2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

B. Genetic Therapy

The present invention contemplates the use of any genetic manipulation for use in modulating the expression of PARP1. Examples of genetic manipulation include, but are not limited to, gene knockout (e.g., removing the PARP1 gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter (e.g., an androgen-responsive promoter)).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subjects in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

C. Antibody Therapy

In some embodiments, the present invention provides antibodies that target prostate tumors that express a gene fusion (e.g., by targeting PARP1). Any suitable antibody (e.g., monoclonal, polyclonal, or synthetic) may be utilized in the therapeutic methods disclosed herein. In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against a gene fusion of the present invention, wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments include agents such as a coagulant, a cytokine, growth factor, bacterial endotoxin or the lipid A moiety of bacterial endotoxin. For example, in some embodiments, therapeutic agents include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeting PARP1. Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

The present invention also includes pharmaceutical compositions and formulations that include the antibody compounds of the present invention as described below.

D. Small Molecules

In some embodiments, small molecule inhibitors of PARP are utilized. Exemplary small molecule PARP inhibitors include, but are not limited to, those described below. 8-hydroxy-2-methylquinazolinone (NU1025) has a structure of

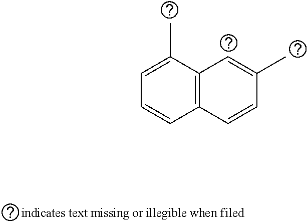

(See e.g., Bowman, K. J., White, A., Golding, B. T., Griffin, R. J., Curtin, N. J. Br. J. Cancer (1998); herein incorporated by reference in its entirety). AZD2281 (Olaparib; CAS No: 937799-91-2,763113-22-0; 4-[3-(4-Cyclopropanecarbonylpiperazine-1-carbonyl)-4-fluorobenzyl]-2H-phthalazin-1-one; AstraZenica) has a chemical formula of C₂₄H₂₃FN₄O₃ and a structure of

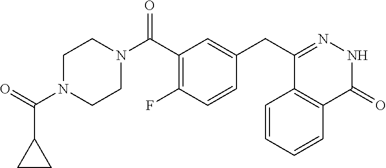

(See e.g., Menear et al., J. Med. Chem., 2008, 51 (20), pp 6581-6591, herein incorporated by reference in its entirety). BSI-201 (4-iodo-3-nitrobenzamide; Iniparib; BiPar Sciences) has a structure of

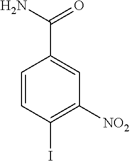

(See e.g., U.S. Pat. No. 7,732,491, herein incorporated by reference in its entirety). ABT-888 (2-[(R)-2-methylpyrrolidin-2-yl]-1H-benzimidazole-4-carboxamide; veliparib) has a structure of

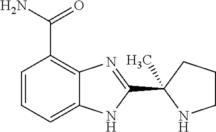

(See e.g., Donawho et al., Clinical Cancer Research May 2007 13:2728-2737, herein incorporated by reference in its entirety). AG014699 has a structure of

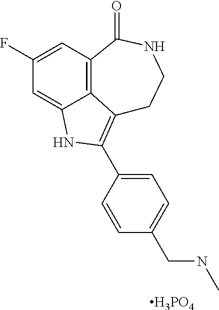

(See e.g., Plummer et al., Clinical Cancer Research December 2008 14; 7917; herein incorporated by reference in its entirety). CEP 9722 hs the structure

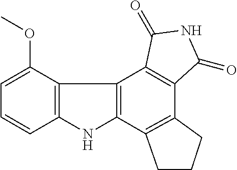

(See e.g., Miknyoczki et al., Mol Cancer Ther August 2007 6; 2290; herein incorporated by reference in its entirety). MK 4827 ((S)-2-(4-(piperidin-3-yl)phenyl)-2H-indazole-7-carboxamide hydrochloride; Merck) has the structure

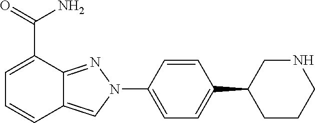

(See e.g., Jones et al., Journal of Medicinal Chemistry (2009), 52(22), 7170-7185; herein incorporated by reference in its entirety). LT-673 (Lead therapeutics, Inc). 3-aminobenzamide is described, for example in DeSoto et al. (Int J Med Sci 2006; 3:117-123 and Jacob, et al. J. Gastroenterol. Hepatol. 2007; 22:738-748).

E. Pharmaceutical Compositions

The present invention further provides pharmaceutical compositions (e.g., comprising pharmaceutical agents that modulate the expression or activity of gene fusions of the present invention). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more antisense compounds and (b) one or more other chemotherapeutic agents that function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

F. Combination Therapy

In some embodiments, the present invention provides therapeutic methods comprising one or more compositions described herein in combination with an additional agent (e.g., a chemotherapeutic agent). The present invention is not limited to a particular chemotherapy agent.

Various classes of antineoplastic (e.g., anticancer) agents are contemplated for use in certain embodiments of the present invention. Anticancer agents suitable for use with embodiments of the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

In some embodiments, exemplary anticancer agents suitable for use in compositions and methods of embodiments of the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; and 23) radiation.

Any oncolytic agent that is routinely used in a cancer therapy context finds use in the compositions and methods of embodiments of the present invention. For example, the U.S. Food and Drug Administration maintains a formulary of oncolytic agents approved for use in the United States. International counterpart agencies to the U.S.F.D.A. maintain similar formularies. The below Table provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

| | | |
|---|---|---|
| Aldesleukin | Proleukin | Chiron Corp., |
| (des-alanyl-1, serine-125 human | | Emeryville, CA |
| interleukin-2) | | |
| Alemtuzumab | Campath | Millennium and |
| (IgG1κ anti CD52 antibody) | | ILEX Partners, |
| | | LP, Cambridge, |
| | | MA |
| Alitretinoin | Panretin | Ligand |
| (9-cis-retinoic acid) | | Pharmaceuticals, |
| | | Inc., San Diego |
| | | CA |
| Allopurinol | Zyloprim | GlaxoSmithKline, |
| (1,5-dihydro-4 H-pyrazolo[3,4- | | Research Triangle |
| d]pyrimidin-4-one monosodium salt) | | Park, NC |
| Altretamine | Hexalen | US Bioscience, |
| (N,N,N′,N′,N″,N″,-hexamethyl-1,3,5- | | West |
| triazine-2,4,6-triamine) | | Conshohocken, |
| | | PA |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a,a,a′,a′- | | Pharmaceuticals, |
| tetramethyl-5-(1H-1,2,4-triazol-1- | | LP, Wilmington, |
| ylmethyl)) | | DE |
| Arsenic trioxide | Trisenox | Cell Therapeutic, |
| | | Inc., Seattle, WA |
| Asparaginase | Elspar | Merck & Co., |
| (L-asparagine amidohydrolase, type EC- | | Inc., Whitehouse |
| 2) | | Station, NJ |
| BCG Live | TICE BCG | Organon Teknika, |
| (lyophilized preparation of an attenuated | | Corp., Durham, |
| strain of Mycobacterium bovis (Bacillus | | NC |
| Calmette-Gukin [BCG], substrain | | |
| Montreal) | | |
| bexarotene capsules | Targretin | Ligand |
| (4-[1-(5,6,7,8-tetrahydro-3,5,5,8,8- | | Pharmaceuticals |
| pentamethyl-2-napthalenyl) ethenyl] | | |
| benzoic acid) | | |
| bexarotene gel | Targretin | Ligand |
| | | Pharmaceuticals |
| Bleomycin | Blenoxane | Bristol-Myers |
| (cytotoxic glycopeptide antibiotics | | Squibb Co., NY, |
| produced by Streptomyces verticillus; | | NY |
| bleomycin A₂ and bleomycin B₂) | | |
| Capecitabine | Xeloda | Roche |
| (5′-deoxy-5-fluoro-N- | | |
| [(pentyloxy)carbonyl]-cytidine) | | |
| Carboplatin | Paraplatin | Bristol-Myers |
| (platinum, diammine [1,1- | | Squibb |
| cyclobutanedicarboxylato(2-)-0,0′]-,(SP- | | |
| 4-2)) | | |
| Carmustine | BCNU, BiCNU | Bristol-Myers |
| (1,3-bis(2-chloroethyl)-1-nitrosourea) | | Squibb |
| Carmustine with Polifeprosan 20 Implant | Gliadel Wafer | Guilford |
| | | Pharmaceuticals, |
| | | Inc., Baltimore, |
| | | MD |
| Celecoxib | Celebrex | Searle |
| (as 4-[5-(4-methylphenyl)-3- | | Pharmaceuticals, |
| (trifluoromethyl)-1H-pyrazol-1-yl] | | England |
| benzenesulfonamide) | | |
| Chlorambucil | Leukeran | GlaxoSmithKline |
| (4- | | |
| [bis(2chlorethyl)amino]benzenebutanoic | | |
| acid) | | |
| Cisplatin | Platinol | Bristol-Myers |
| (PtCl₂H₆N₂) | | Squibb |
| Cladribine | Leustatin, 2- | R.W. Johnson |
| (2-chloro-2′-deoxy-b-D-adenosine) | CdA | Pharmaceutical |
| | | Research |
| | | Institute, Raritan, |
| | | NJ |
| Cyclophosphamide | Cytoxan, | Bristol-Myers |
| (2-[bis(2-chloroethyl)amino] tetrahydro- | Neosar | Squibb |
| 2H-13,2-oxazaphosphorine 2-oxide | | |
| monohydrate) | | |
| Cytarabine | Cytosar-U | Pharmacia & |
| (1-b-D-Arabinofuranosylcytosine, | | Upjohn Company |
| C₉H₁₃N₃O₅) | | |
| cytarabine liposomal | DepoCyt | Skye |
| | | Pharmaceuticals, |
| | | Inc., San Diego, |
| | | CA |
| Dacarbazine | DTIC-Dome | Bayer AG, |
| (5-(3,3-dimethyl-l-triazeno)-imidazole-4- | | Leverkusen, |
| carboxamide (DTIC)) | | Germany |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., |
| (recombinant peptide) | | Thousand Oaks, |
| | | CA |
| daunorubicin liposomal | DanuoXome | Nexstar |
| ((8S-cis)-8-acetyl-10-[(3-amino-2,3,6- | | Pharmaceuticals, |
| trideoxy-a-L-lyxo-hexopyranosyl)oxy]- | | Inc., Boulder, CO |
| 7,8,9,10-tetrahydro-6,8,11-trihydroxy-1- | | |
| methoxy-5,12-naphthacenedione | | |
| hydrochloride) | | |
| Daunorubicin HCl, daunomycin | Cerubidine | Wyeth Ayerst, |
| ((1S,3S)-3-Acetyl-1,2,3,4,6,11- | | Madison, NJ |
| hexahydro-3,5,12-trihydroxy-10- | | |
| methoxy-6,11-dioxo-1-naphthacenyl 3- | | |
| amino-2,3,6-trideoxy-(alpha)-L-lyxo- | | |
| hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & |
| ((S)-4,4′-(1-methyl-1,2-ethanediyl)bis- | | Upjohn Company |
| 2,6-piperazinedione) | | |
| Docetaxel | Taxotere | Aventis |
| ((2R,3S)-N-carboxy-3-phenylisoserine, | | Pharmaceuticals, |
| N-tert-butyl ester, 13-ester with 5b-20- | | Inc., Bridgewater, |
| epoxy-12a,4,7b,10b,13a-hexahydroxytax- | | NJ |
| 11-en-9-one 4-acetate 2-benzoate, | | |
| trihydrate) | | |
| Doxorubicin HCl | Adriamycin, | Pharmacia & |
| (8S,10S)-10-[(3-amino-2,3,6-trideoxy-a- | Rubex | Upjohn Company |
| L-lyxo-hexopyranosyl)oxy]-8-glycolyl- | | |
| 7,8,9,10-tetrahydro-6,8,11-trihydroxy-1- | | |
| methoxy-5,12-naphthacenedione | | |
| hydrochloride) | | |
| doxorubicin | Adriamycin | Pharmacia & |
| | PFS Intravenous | Upjohn Company |
| | injection | |
| doxorubicin liposomal | Doxil | Sequus |
| | | Pharmaceuticals, |
| | | Inc., Menlo park, |
| | | CA |
| dromostanolone propionate | Dromostanolone | Eli Lilly & |
| (17b-Hydroxy-2a-methyl-5a-androstan-3- | | Company, |
| one propionate) | | Indianapolis, IN |
| dromostanolone propionate | Masterone | Syntex, Corp., |
| | injection | Palo Alto, CA |
| Elliott's B Solution | Elliott's B | Orphan Medical, |
| | Solution | Inc |
| Epirubicin | Ellence | Pharmacia & |
| ((8S-cis)-10-[(3-amino-2,3,6-trideoxy-a- | | Upjohn Company |
| L-arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & |
| (estra-1,3,5(10)-triene-3,17- | | Upjohn Company |
| diol(17(beta))-, 3-[bis(2- | | |
| chloroethyl)carbamate] 17-(dihydrogen | | |
| phosphate), disodium salt, monohydrate, | | |
| or estradiol 3-[bis(2- | | |
| chloroethyl)carbamate] 17-(dihydrogen | | |
| phosphate), disodium salt, monohydrate) | | |
| Etoposide phosphate | Etopophos | Bristol-Myers |
| (4′-Demethylepipodophyllotoxin 9-[4,6- | | Squibb |
| O-(R)-ethylidene-(beta)-D- | | |
| glucopyranoside], 4′-(dihydrogen | | |
| phosphate)) | | |
| etoposide, VP-16 | Vepesid | Bristol-Myers |
| (4′-demethylepipodophyllotoxin 9-[4,6-0- | | Squibb |
| (R)-ethylidene-(beta)-D- | | |
| glucopyranoside]) | | |
| Exemestane | Aromasin | Pharmacia & |
| (6-methylenandrosta-1,4-diene-3,17- | | Upjohn Company |
| dione) | | |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex |
| (fluorinated nucleotide analog of the | | Laboratories, Inc., |
| antiviral agent vidarabine, 9-b-D- | | Cedar Knolls, NJ |
| arabinofuranosyladenine (ara-A)) | | |
| Fluorouracil, 5-FU | Adrucil | ICN |
| (5-fluoro-2,4(1H,3H)-pyrimidinedione) | | Pharmaceuticals, |
| | | Inc., Humacao, |
| | | Puerto Rico |
| Fulvestrant | Faslodex | IPR |
| (7-alpha-[9-(4,4,5,5,5-penta | | Pharmaceuticals, |
| fluoropentylsulphinyl) nonyl]estra-1,3,5- | | Guayama, Puerto |
| (10)-triene-3,17-beta-diol) | | Rico |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| (acetate salt of [D- | | Pharmaceuticals |
| Ser(But)⁶,Azgly¹⁰]LHRH; pyro-Glu-His- | | |
| Trp-Ser-Tyr-D-Ser(But)-Leu-Arg-Pro- | | |
| Azgly-NH2 acetate [C₅₉H₈₄N₁₈O₁₄•(C₂H₄O₂)ₓ | | |
| Hydroxyurea | Hydrea | Bristol-Myers |
| | | Squibb |
| Ibritumomab Tiuxetan | Zevalin | Biogen IDEC, |
| (immunoconjugate resulting from a | | Inc., Cambridge |
| thiourea covalent bond between the | | MA |
| monoclonal antibody Ibritumomab and | | |
| the linker-chelator tiuxetan [N-[2- | | |
| bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & |
| (5,12-Naphthacenedione, 9-acetyl-7-[(3- | | Upjohn Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride, (7S-cis)) | | |
| Ifosfamide | IFEX | Bristol-Myers |
| (3-(2-chloroethyl)-2-[(2- | | Squibb |
| chloroethyl)amino]tetrahydro-2H-1,3,2- | | |
| oxazaphosphorine 2-oxide) | | |
| Imatinib Mesilate | Gleevec | Novartis AG, |
| (4-[(4-Methyl-1-piperazinyl)methyl]-N- | | Basel, |
| [4-methyl-3-[[4-(3-pyridinyl)-2- | | Switzerland |
| pyrimidinyl]amino]-phenyl]benzamide | | |
| methanesulfonate) | | |
| Interferon alfa-2a | Roferon-A | Hoffmann-La |
| (recombinant peptide) | | Roche, Inc., |
| | | Nutley, NJ |
| Interferon alfa-2b | Intron A | Schering AG, |
| (recombinant peptide) | (Lyophilized | Berlin, Germany |
| | Betaseron) | |
| Irinotecan HCl | Camptosar | Pharmacia & |
| ((4S)-4,11-diethyl-4-hydroxy-9-[(4- | | Upjohn Company |
| piperi-dinopiperidino)carbonyloxy]-1H- | | |
| pyrano[3′,4′:6,7] indolizino[1,2-b] | | |
| quinoline-3,14(4H,12H) dione | | |
| hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5- | Leucovorin | Seattle, WA |
| formyl1,4,5,6,7,8 hexahydro4oxo6- | | |
| pteridinyl)methyl]amino]benzoyl], | | |
| calcium salt (1:1)) | | |
| Levamisole HCl | Ergamisol | Janssen Research |
| ((−)-(S)-2,3,5,6-tetrahydro-6- | | Foundation, |
| phenylimidazo [2,1-b] thiazole | | Titusville, NJ |
| monohydrochloride C₁₁H₁₂N₂S•HCl) | | |
| Lomustine | CeeNU | Bristol-Myers |
| (1-(2-chloro-ethyl)-3-cyclohexyl-1- | | Squibb |
| nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers |
| 17α(acetyloxy)-6-methylpregna-4,6- | | Squibb |
| diene-3,20-dione | | |
| Melphalan, L-PAM | Alkeran | GlaxoSmithKline |
| (4-[bis(2-chloroethyl) amino]-L- | | |
| phenylalanine) | | |
| Mercaptopurine, 6-MP | Purinethol | GlaxoSmithKline |
| (1,7-dihydro-6 H-purine-6-thione | | |
| monohydrate) | | |
| Mesna | Mesnex | Asta Medica |
| (sodium 2-mercaptoethane sulfonate) | | |
| Methotrexate | Methotrexate | Lederle |
| (N-[4-[[(2,4-diamino-6- | | Laboratories |
| pteridinyl)methyl]methylamino]benzoyl]- | | |
| L-glutamic acid) | | |
| Methoxsalen | Uvadex | Therakos, Inc., |
| (9-methoxy-7H-furo[3,2-g][1]- | | Way Exton, Pa |
| benzopyran-7-one) | | |
| Mitomycin C | Mutamycin | Bristol-Myers |
| | | Squibb |
| mitomycin C | Mitozytrex | SuperGen, Inc., |
| | | Dublin, CA |
| Mitotane | Lysodren | Bristol-Myers |
| (1,1-dichloro-2-(o-chlorophenyl)-2-(p- | | Squibb |
| chlorophenyl) ethane) | | |
| Mitoxantrone | Novantrone | Immunex |
| (1,4-dihydroxy-5,8-bis[[2-[(2- | | Corporation |
| hydroxyethyl)amino]ethyl]amino]-9,10- | | |
| anthracenedione dihydrochloride) | | |
| Nandrolone phenpropionate | Durabolin-50 | Organon, Inc., |
| | | West Orange, NJ |
| Nofetumomab | Verluma | Boehringer |
| | | Ingelheim Pharma |
| | | KG, Germany |
| Oprelvekin | Neumega | Genetics Institute, |
| (IL-11) | | Inc., Alexandria, |
| | | VA |
| Oxaliplatin | Eloxatin | Sanofi |
| (cis-[(1R,2R)-1,2-cyclohexanediamine- | | Synthelabo, Inc., |
| N,N′] [oxalato(2-)-O,O′] platinum) | | NY, NY |
| Paclitaxel | TAXOL | Bristol-Myers |
| (5β,20-Epoxy-1,2a,4,7β,10β,13a- | | Squibb |
| hexahydroxytax-11-en-9-one 4,10- | | |
| diacetate 2-benzoate 13-ester with (2R,3S)-N- | | |
| benzoyl-3-phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen | Enzon |
| ((monomethoxypolyethylene glycol | (Pegademase | Pharmaceuticals, |
| succinimidyl) 11-17-adenosine | Bovine) | Inc., Bridgewater, |
| deaminase) | | NJ |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant | | |
| methionyl human G-CSF (Filgrastim) | | |
| and monomethoxypolyethylene glycol) | | |
| Pentostatin | Nipent | Parke-Davis |
| | | Pharmaceutical |
| | | Co., Rockville, |
| | | MD |
| Pipobroman | Vercyte | Abbott |
| | | Laboratories, |
| | | Abbott Park, IL |
| Plicamycin, Mithramycin | Mithracin | Pfizer, Inc., NY, |
| (antibiotic produced by Streptomyces | | NY |
| plicatus) | | |
| Porfimer sodium | Photofrin | QLT |
| | | Phototherapeutics, |
| | | Inc., Vancouver, |
| | | Canada |
| Procarbazine | Matulane | Sigma Tau |
| (N-isopropyl-μ-(2-methylhydrazino)-p- | | Pharmaceuticals, |
| toluamide monohydrochloride) | | Inc., |
| | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi- |
| (recombinant peptide) | | Synthelabo, Inc., |
| Rituximab | Rituxan | Genentech, Inc., |
| (recombinant anti-CD20 antibody) | | South San |
| | | Francisco, CA |
| Sargramostim | Prokine | Immunex Corp |
| (recombinant peptide) | | |
| Streptozocin | Zanosar | Pharmacia & |
| (streptozocin 2-deoxy-2- | | Upjohn Company |
| [[(methylnitrosoamino)carbonyl]amino]- | | |
| a(and b)-D-glucopyranose and 220 mg | | |
| citric acid anhydrous) | | |
| Talc | Sclerosol | Bryan, Corp., |
| (Mg₃Si₄O₁₀(OH)₂) | | Woburn, MA |
| Tamoxifen | Nolvadex | AstraZeneca |
| ((Z)2-[4-(1,2-diphenyl-1-butenyl) | | Pharmaceuticals |
| phenoxy]-N,N-dimethylethanamine 2- | | |
| hydroxy-1,2,3-propanetricarboxylate | | |
| (1:1)) | | |
| Temozolomide | Temodar | Schering |
| (3,4-dihydro-3-methyl-4-oxoimidazo[5,1- | | |
| d]-as-tetrazine-8-carboxamide) | | |
| Teniposide, VM-26 | Vumon | Bristol-Myers |
| (4′-demethylepipodophyllotoxin 9-[4,6-0- | | Squibb |
| (R)-2-thenylidene-(beta)-D- | | |
| glucopyranoside]) | | |
| Testolactone | Teslac | Bristol-Myers |
| (13-hydroxy-3-oxo-13,17-secoandrosta- | | Squibb |
| 1,4-dien-17-oic acid [dgr]-lactone) | | |
| Thioguanine, 6-TG | Thioguanine | GlaxoSmithKline |
| (2-amino-1,7-dihydro-6 H-purine-6- | | |
| thione) | | |
| Thiotepa | Thioplex | Immunex |
| (Aziridine, 1,1′,1″- | | Corporation |
| phosphinothioylidynetris-, or Tris (1- | | |
| aziridinyl) phosphine sulfide) | | |
| Topotecan HCl | Hycamtin | GlaxoSmithKline |
| ((S)-10-[(dimethylamino) methyl]-4- | | |
| ethyl-4,9-dihydroxy-1H-pyrano[3′,4′: | | |
| 6,7] indolizino [1,2-b] quinoline-3,14- | | |
| (4H,12H)-dione monohydrochloride) | | |
| Toremifene | Fareston | Roberts |
| (2-(p-[(Z)-4-chloro-1,2-diphenyl-1- | | Pharmaceutical |
| butenyl]-phenoxy)-N,N- | | Corp., Eatontown, |
| dimethylethylamine citrate (1:1)) | | NJ |
| Tositumomab, I 131 Tositumomab | Bexxar | Corixa Corp., |
| (recombinant murine immunotherapeutic | | Seattle, WA |
| monoclonal IgG₂ₐ lambda anti-CD20 | | |
| antibody (I 131 is a | | |
| radioimmunotherapeutic antibody)) | | |
| Trastuzumab | Herceptin | Genentech, Inc |
| (recombinant monoclonal IgG₁ kappa | | |
| anti-HER2 antibody) | | |
| Tretinoin, ATRA | Vesanoid | Roche |
| (all-trans retinoic acid) | | |
| Uracil Mustard | Uracil Mustard | Roberts Labs |
| | Capsules | |
| Valrubicin, N-trifluoroacetyladriamycin- | Valstar | Anthra --> |
| 14-valerate | | Medeva |
| ((2S-cis)-2-[1,2,3,4,6,11-hexahydro- | | |
| 2,5,12-trihydroxy-7 methoxy-6,11-dioxo- | | |
| [[4 2,3,6-trideoxy-3-[(trifluoroacetyl)- | | |
| amino-α-L-lyxo-hexopyranosyl]oxyl]-2- | | |
| naphthacenyl]-2-oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2) (salt)]) | | |
| Zoledronate, Zoledronic acid | Zometa | Novartis |
| ((1-Hydroxy-2-imidazol-1-yl- | | |
| phosphonoethyl) phosphonic acid | | |
| monohydrate) | | |

### III. Drug Screening Applications

In some embodiments, the present invention provides drug screening assays (e.g., to screen for anticancer drugs). The screening methods of the present invention utilize PARP1. For example, in some embodiments, the present invention provides methods of screening for compounds that alter (e.g., decrease) the expression or activity of PARP1. The compounds or agents may interfere with transcription, by interacting, for example, with the promoter region. The compounds or agents may interfere with mRNA produced from the fusion (e.g., by RNA interference, antisense technologies, etc.). The compounds or agents may interfere with pathways that are upstream or downstream of the biological activity of PARP1. In some embodiments, candidate compounds are antisense or interfering RNA agents (e.g., oligonucleotides) directed against PARP1. In other embodiments, candidate compounds are antibodies or small molecules that specifically bind to a PARP1 regulator or expression products inhibit its biological function.

In one screening method, candidate compounds are evaluated for their ability to alter PARP1 expression by contacting a compound with a cell expressing a gene fusion and then assaying for the effect of the candidate compounds on expression. In some embodiments, the effect of candidate compounds on expression of PARP1 is assayed for by detecting the level of gene fusion mRNA expressed by the cell. mRNA expression can be detected by any suitable method.

In other embodiments, the effect of candidate compounds on expression of PARP1 is assayed by measuring the level of polypeptide encoded by PARP1. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to PARP1, have an inhibitory (or stimulatory) effect on, for example, PARP1 expression or activity, or have a stimulatory or inhibitory effect on, for example, the expression or activity of a PARP1 substrate. Compounds thus identified can be used to modulate the activity of target gene products (e.g., PARP1) either directly or indirectly in a therapeutic protocol, to elaborate the biological function of the target gene product, or to identify compounds that disrupt normal target gene interactions. Compounds that inhibit the activity or expression of PARP1 are useful in the treatment of proliferative disorders, e.g., cancer, particularly prostate cancer.

In one embodiment, the invention provides assays for screening candidate or test compounds that are substrates of a PARP1 protein or polypeptide or a biologically active portion thereof. In another embodiment, the invention provides assays for screening candidate or test compounds that bind to or modulate the activity of a gene fusion protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. Natl. Acad. Sci. 87:6378-6382 [1990]; Felici, J. Mol. Biol. 222:301 [1991]).

### EXPERIMENTAL

The following examples are provided in order to demonstrate and further illustrate certain preferred embodiments and aspects of the present invention and are not to be construed as limiting the scope thereof.

### Example 1

### A. Experimental Procedures

### Protein Identification by LC-Tandem MS

Lyophilized peptides from each gel fraction were reconstituted in 1% acetic acid in 5% acetonitrile for reverse phase separation on-line to the nano-spray equipped LTQ-XL ion trap mass spectrometer (ThermoFisher, San Jose, Calif.). Each fraction was loaded using an autosampler (SPARK, Michrom Bioresources, CA) and separated on an Aquasil C18 Picofrit column, (15 cm×75 μm i.d., 15 μM tip) (New Objectives, Woburn Mass.). Peptides were eluted over 60 minutes with an increasing linear gradient of acetonitrile at a flow rate of 300 mL/minute. To identify the eluting peptides, the mass spectrometer was operated in a data-dependent MS/MS mode (m/z 400-2000), in which the top five ions were subjected to MS/MS at 35% of 1 V normalized collision induced disassociation. Dynamic mass exclusion was enabled with a repeat count of 2 for 2 minutes and a list size of 200 m/z.

### Immunoprecipitation and Western Blot

Cell lysates (0.5-1.0 mg) were pre-cleared and then incubated with ethidium bromide as previously described (Yano, K., et al. EMBO reports 9, 91-96 (2008) and analyzed by SDS-PAGE Western blot analysis as in Cao, Q., et al. (Oncogene 27, 7274-7284 (2008)).

### Small RNA Interference

Knockdowns of specific genes were accomplished by RNA interference using commercially available siRNA duplexes for DNA-PKcs, ATM, ATR, PARP1-1, XRCC4 and DNA Ligase4 (Dharmacon, Lafayette, Colo.) or as previously described for ERG (Tomlins et al., 2008, supra). At least 4 independent siRNAs were screened for knockdown efficiency against each target and the best siRNA was selected, in some cases only one siRNA was identified. Transfections were performed with OptiMEM (Invitrogen) and oligofectamine (Invitrogen) as previously described (Varambally, S., et al. Science (New York, N.Y. 322, 1695-1699 (2008)). Sequences of siRNAs are shown in Table 5.

### Chromatin Immunoprecipitation

VCaP or RWPE cells were grown in complete medium and ChIP was carried out as previously described (Yu, J., et al. Cancer cell 12, 419-431 (2007)) using antibodies against ERG (Santa Cruz, #sc-354), DNA-PKcs (BD Biosciences, #610805), pDNA-PKcs (T2609) (Santa Cruz, #sc-101664), Ku80 (Cell Signaling, #2180), Ku70 (BD Biosciences, #611892), PARP1-1 (Santa Cruz, #sc-8007), rabbit IgG (Santa Cruz, #sc-2027) and mouse IgG (Santa Cruz, #sc-2025). Briefly, cultured cells were crosslinked with 1% formaldehyde for 10 min and the crosslinking was inactivated by 0.125 M glycine for 5 min at room temperature (RT). Cells were then rinsed with 1×PBS twice and frozen in 1×PBS+10 μl/ml PMSF+phosphatase inhibitor cocktail (Calbiochem) for 30 min. The following steps were performed at 4° C. Cell pellets were resuspended and incubated in cell lysis buffer+10 μl/ml PMSF and protease inhibitor (Roche) for 10 min. Nuclei pellets were spun down at 5,000×g for 5 min, resuspended in nuclear lysis buffer, and then incubated for another 10 min. Chromatin was sonicated to an average length of 600 bp and then centrifuged at 14,000×g for 10 min to remove the debris. Supernatants containing chromatin fragments were incubated with agarose/protein A or G beads (Upstate) for 15 min and centrifuged at 5,000×g for 5 min to remove the nonspecific binding. To immunoprecipitate protein/chromatin complexes, the supernatants were incubated with 3-5 μg of antibody or IgG overnight, 50 μl of agarose/protein A or G beads was then added and the reaction mix was incubated for another 1 hour. Beads were washed twice with 1× dialysis buffer and four times with IP buffer. The antibody/protein/DNA complexes were eluted with 150 μl IP elution buffer twice. To reverse the crosslinks, the complexes were incubated in elution buffer+10 μg RNase A and 0.3 M NaCl at 67° C. for 4 hours. DNA/proteins were precipitated with ethanol, air-dried, and dissolved in 100 μl of TE. Proteins were then digested by proteinase K at 45° C. for 1 hour and DNA was purified with QIAGEN PCR column and eluted with 30 μl EB. The final ChIP yield was 10-30 ng for each antibody. QPCR is described below and primers were either previously described (Tomlins et al., 2008, supra) or are in Table 4.

### Luciferase Reporter Assay

Luciferase reporter assays were performed as previously descried (Cao et al., 2008, supra). Briefly, RWPE cells were infected with siRNA as indicated 6 hours before the addition of either ERG or control LACZ adenovirus. The PLA1A promoter reporter construct was co-transfected along with pRL-TK (internal control). Twenty four hours postinfection, cells were harvested with passive lysis buffer and luciferase activity was monitored using dual luciferase assay system (Promega, Madison, Wis.) following manufacturer's instructions. The PLA1A promoter fragment was PCR amplified using a genomic BAC clone as template with the forward primer (5′-CCCCATTGACTTGCCTAGAA (SEQ ID NO:1)) and reverse primer (5′-GGCTTTTAGGGGATCTTCCA (SEQ ID NO:2) and subcloned into pGL4.14 vector (Promega) using XhoI and Hind3 enzymes.

### Quantitative Real-Time PCR Assays

Total RNA was isolated from VCAP, RWPE or PrEC cells that were transfected with siRNA as indicated (Qiagen). Quantitative PCR (QPCR) was performed using SYBR Green dye on an Applied Biosystems 7300 Real Time PCR system (Applied Biosystems, Foster City, Calif.) as described (Tomlins, S. A., et al. Science (New York, N.Y. 310, 644-648 (2005)). Briefly, 1 μg of total RNA was reverse transcribed into cDNA using SuperScript 111 (Invitrogen, Carlsbad, Calif.) in the presence of random primers (Invitrogen). All reactions were performed in triplicate with SYBR Green Master Mix (Applied Biosystems) plus 25 ng of both the forward and reverse primer according to the manufacturer's recommended thermocycling conditions, and then subjected to melt curve analysis. Threshold levels for each experiment were set during the exponential phase of the QPCR reaction using Sequence Detection Software version 1.2.2 (Applied Biosystems). The relative quantity of the target gene was completed for each sample using the ΔΔCt method by the comparing mean Ct of the gene to the average Ct of the housekeeping gene, β-Actin8. All oligonucleotide primers were synthesized by Integrated DNA Technologies (Coralville, Iowa). The primer sequences for the transcript analyzed were either previously described (Tomlins et al., 2008, supra) or are provided in Table 4.

### Basement Membrane Matrix Invasion Assays

For invasion assays, the prostate cell lines RWPE-1 and VCaP were transfected with siRNA or negative controls as indicated. NU7026 (Sigma) and NU1025 (Calbiochem) were dissolved in DMSO and stored at −20° C. in the dark. Forty-eight hours post-transfection/transduction, cells were seeded onto the basement membrane matrix (EC matrix, Chemicon, Temecula, Calif.) in the chamber insert with 8.0 μM pores of a 24-well culture plate in serum free media. Cells were attracted to the lower chamber by the addition of complete media as a chemoattractant. After 48 hours incubation at 37° C. with 5% CO₂, the non-invading cells and EC matrix were gently removed with a cotton swab. Invasive cells, which were located on the lower side of the membrane, were stained with crystal violet, air dried and photographed. To quantify the relative number of invaded cells, colorimetric assays were performed by treating the inserts with 150 μl of 10% acetic acid (v/v) and measuring absorbance of each condition at 560 nm using a spectrophotometer (GE Healthcare).

### CAM Assays

The CAM assay was performed as described previously (Zijlstra, A., et al., Cancer research 62, 7083-7092 (2002)). Briefly, fertilized eggs were incubated in a rotary humidified incubator at 38° C. with for 10 days. After releasing the CAM by applying mild amount of low pressure to the hole over the air sac and cutting a square 1-cm² window encompassing a second hole near the allantoic vein, cultured human cells that had been pre-treated with siRNA as indicated were detached by trypsinization and re-suspended in complete medium prior to implantation of 2×10⁶ cells adjacent to the mesenchyme in each egg. The windows were subsequently, sealed and the eggs were returned to a stationary incubator. Cells were then treated as indicated with 40 mg/kg body weight Olaparib which was administered on day 13, 15 and 17. Day 18 embryos were sacrificed. Tumors were then excised and weighed. For intravasation experiments, implanted eggs were treated 6 hours after cell inoculation and the lower CAM was isolated after 72 hours. After incubation of either the metastasis or intravasation assays, the extra-embryonic tumor and CAM, as well as the embryonic liver were harvested and analyzed for the presence of tumor cells by quantitative human alu-specific PCR. Genomic DNA from lower CAM and lungs were prepared using Puregene DNA purification system (Qiagen). Quantification of human cells in the extracted DNA was done as described¹⁴. Fluorogenic TaqMan qPCR probes were applied as above, and DNA copy numbers were quantified.

### VCaP, PC3-Control and PC3-ERG Xenograft Models

Four weeks old male Balb C nu/nu mice were purchased from Charles River, Inc. (Charles River Laboratory, Wilmington, Mass.). VCaP (2×10⁶ cells), PC3-luciferase-control (1×10⁵ cells) or PC3-luciferase-ERG (1×10⁵ cells) stable cells were resuspended in 100 μl of saline with 50% Matrigel (BD Biosciences, Becton Drive, NJ) and were implanted subcutaneously into the left flank region of the mice. Mice were anesthetized using a cocktail of xylazine (80-120 mg/kg, IP) and ketamine (10 mg/kg, IP) for chemical restraint before tumor implantation. All tumors were staged for three week before starting the drug treatment. After week 3 for VCaP cells or after 48 hours for PC3 cells, mice (10 per treatment group) were treated with NU7026 (25 mg/kg, IP), Olaparib (40 mg/kg, IP) daily five times a week. Olaparib was obtained from Axon Medchem (Groningen, The Netherlands). Growth in tumor volume was recorded weekly by using digital calipers and tumor volumes were calculated using the formula (π/6) (L×W2), where L=length of tumor and W=width. Loss of body weight during the course of the study was also monitored weekly. All procedures involving mice were approved by the University Committee on Use and Care of Animals (UCUCA) at the University of Michigan and conform to their relevant regulatory standards.

### Immunofluorescence

Cells were seeded at 25,000 cells/mL in 4-well chamber slide 24 hours prior to the addition of siRNA or drug as indicated. Merbarone (Sigma) was dissolved in DMSO and stored at 4° C. Cells were then washed once in PBS, fixed for 15 minutes in 100% methanol, washed in PBS, permeabilized for 5 minutes in PBS containing 0.2% Triton-X, washed twice in PBS, blocked for 30 minutes in PBS containing 0.5% donkey serum (Sigma, St. Louis, Mo.), held for 45 minutes in PBS containing 0.5% donkey serum and primary antibody: anti-γ-H2A.X mouse monoclonal (Millipore Cat #05-636), anti-γ-H2A.X rabbit polyclonal (Cell Signaling, Cat #9719, Danvers, Mass.), and anti-53BP1 rabbit polyclonal (Cell Signaling, Cat #4937, Danvers, Mass.) washed 3 times with PBS, held for 30 minutes in PBS containing 0.5% donkey serum and secondary antibody: Alexa Fluor 488-anti-Mouse (Invitrogen) or Alexa Fluor 594-anti-Rabbit (Invitrogen). Cells were then stained with DAPI for 5 minutes and slides were mounted 12 hours prior to analysis using Vectashield (Vector laboratories, Burlingame, Calif.). Images were taken using 100× oil lens on an Olympus Confocal microscope at the University of Michigan microscopy imaging lab.

### Statistical Analysis

The data are expressed as the means±SE of the means. Student's t test was used (* P<0.05 and ** P<0.01), and all statistical tests are two-tailed, unless otherwise indicated.

### Cell Lines

PC3 prostate cancer cell lines were grown in RPMI 1640 (Invitrogen, Carlsbad, Calif.) and VCaP cells in DMEM with Glutamax (Invitrogen) both supplemented with 10% FBS (Invitrogen) in 5% CO₂ cell culture incubator. The immortalized prostate cell line RWPE-1 was grown in Keratinocyte media with L-glutamine (Invitrogen) supplemented with 2.5 μg EGF (Invitrogen) and 25 mg Bovine Pituitary Extract (Invitrogen). All cultures were also maintained with 50 units/ml of Penicillin/streptomycin (Invitrogen). The genetic identity of each cell line was confirmed by genotyping samples. Briefly, DNA samples were diluted to 0.10 ng/μl and analyzed in the University of Michigan DNA sequencing Core using the Profiler Plus PCR Amplification Kit (Applied Biosystems, Foster City, Calif.) according to the manufacturer's protocol. The 9 loci D3S1358, D5S818, D7S820, D8S1179, D13S317, D18S51, D21S11, FGA, vWA and the Amelogenin locus were analyzed and compared to ladder control samples as previously described (Brenner, J. C., et al. Head & neck (2009). Lentiviruses were generated by the University of Michigan Vector Core. PC3 cells were infected with lentiviruses expressing pLentilox-CMV-ERG or pLentilox-CMVLACZ control and stable cell lines were selected by sorting at the University of Michigan flow cytometry core. Stable infection was monitored by confirming GFP expression every three days. Likewise, PC3-luciferase cells were created by transduction of pLentilox-CMV-Luciferase available from the University of Michigan Vector Core. Stable RWPE-ERG and RWPE-LACZ cells were created and described previously (Tomlins et al., 2008, supra).

### Mass Spectrometry

IP eluate was resolved with SDS-PAGE and visualized with silver stain (PROTSIL-2, Sigma). Each gel lane, experimental and control was excised into 16 equal sized and corresponding pieces for in-gel trypsin digestion. Briefly, gel pieces were destained, treated with 10 mM DTT followed immediately with 50 mM iodoacetaminde to reduce and alkylate cysteine residues, and then incubated with trypsin (1:20) enzyme:protein (w:w) overnight at 37° C. (Promega, Madison, Wis.). Peptides were then extracted from the gel, lyophilized to dryness, and stored at −80° C. until further analysis.

### Database Searching

Raw spectra files were converted to mzXML format using an in-house installation of ReAdW (version 4.0.2), and searched with X!Tandem (The Global Proteome Machine Organization; version 2007.07.01.1) on a decoy database that contained the forward human IPI sequences concatenated to the reversed human IPI sequences (version 3.41) plus cRAP database of common contaminants. The database search used trypsin enzyme specificity, a mass error of 3 Da on parent ion and 0.8 Da on fragment ions, a maximum of one missed cleavage, and variable modifications of oxidation on methionine and carbamidomethlyation on cysteines. The X!Tandem database search results were analyzed and validated using Trans-Proteomic Pipeline (TPP) software (version 4.0.2). All entries with a peptide probability >90% and a protein probability error rate <5% with at least two unique peptides were considered high confidence identifications.

Proteins that contained similar peptides and could not be differentiated based on MS/MS analysis alone were grouped to satisfy the principles of parsimony. Only individual protein isoforms are reported and those proteins identified with high confidence unique to experimental when compared with matched control. As a control, epitope-tagged beta-galactosidase (LACZ) was run in parallel and immunoprecipitated in each replicate experiment. Non-specific interactions identified in the vector controls were manually removed from the experimental protein listing to generate a final list of genuine ERG interactions found in both HEK293 and VCaP cells over eight biological replicates.

### Immunoprecipitation

Cells were lysed in Triton X-100 lysis buffer (20 mM MOPS, pH 7.0, 2 mM EGTA, 5 mM EDTA, 30 mM sodium fluoride, 60 mM β-glycerophosphate, 20 mM sodium pyrophosphate, 1 mM sodium orthovanadate, 1% Triton X-100, protease inhibitor cocktail (Roche, #14309200)). Cell lysates (0.5-1.0 mg) were then pre-cleaned with protein A/G agarose beads (Santa Cruz, # sc-2003) by incubation for 1 hour with shaking at room temperature followed by centrifugation at 2000×g for 1 minute. Lysates were incubated with ethidium bromide (Sigma) as previously described (Yano et al., EMBO reports 9, 91-96 (2008)). Then, after adding 2 μg antibody, lysates were incubated at 4° C. for 4 hours with shaking prior to addition of 20 μL protein A/G agarose beads. The mixture was then incubated with shaking at 4° C. for another 4 hours prior to washing the lysate-bead precipitate (centrifugation at 2000×g for 1 minute) 3 times in Triton X-100 lysis buffer. Beads were finally precipitated by centrifugation, resuspended in 25 μL of 2× loading buffer and boiled at 80° C. for 10 minutes for separation the protein and beads. Samples were then analyzed by SDS-PAGE Western blot analysis as described below.

### Immunoblot Analysis

The cell lines were plated in two wells of a 6-well plate at 250,000 cells/mL 24 hours prior to harvesting by trypsinization. Pellets were then flash frozen, briefly sonicated and homogenized in NP40 lysis buffer (50 mM Tris-HCl, 1% NP40, pH 7.4, Sigma, St. Louis, Mo.), and complete proteinase inhibitor mixture (Roche, Indianapolis, Ind.). Ten micrograms of each protein extract were boiled in sample buffer, size fractionated by SDS-PAGE, and transferred onto Polyvinylidene Difluoride membrane (GE Healthcare, Piscataway, N.J.). The membrane was then incubated overnight at 4° C. in blocking buffer (Tris-buffered saline, 0.1% Tween (TBS-T), 5% nonfat dry milk) and incubated for 4 hours at room temperature with the following: anti-DNA-PKcs mouse monoclonal (1:1000 in blocking buffer, BD Biosciences #610805, San Jose, Calif.), anti-Ku70 mouse monoclonal (1:1000 in blocking buffer, BD Biosciences #611892), anti-Ku80 rabbit polyclonal (1:1000 in blocking buffer, Cell Signaling Cat #2180S, Danvers, Mass.), anti-ATR rabbit polyclonal (1:1000 in blocking buffer, Cell Signaling Cat #2790), anti-ERG1/2/3 rabbit polyclonal (1:1000 in blocking buffer, Santa Cruz Biotech # sc-354, Santa Cruz, Calif.), anti-PARP1-1 polyclonal (1:1000 in blocking buffer, Santa Cruz Biotech Cat #sc-8007, Santa Cruz, Calif.), anti-XRCC4 mouse polyclonal (1:1000 in blocking buffer, BD Biosciences Cat #611506), anti-LACZ (1:1000 in blocking buffer, GenwayBio Cat#18-732-292258), anti-FLAG rabbit (1:1000 in blocking buffer, Sigma Cat #F7425), anti-V5 mouse monoclonal (1:1000 in blocking buffer, Invitrogen #R-960, Carlsbad, Calif.) and anti-β-Actin mouse monoclonal antibody (1:10000, Cell Signaling, Cat #: 4967). Following a wash with TBS-T, the blot was incubated with horseradish peroxidase-conjugated secondary antibody and the signals visualized by enhanced chemiluminescence system as described by the manufacturer (GE Healthcare).

### Luciferase Reporter Assay

Luciferase reporter assays were performed as previously descried (Cao et al., 2008, supra). Briefly, RWPE cells were infected with siRNA as indicated 6 hours before the addition of either ERG or control LACZ adenovirus. The PLA1A promoter reporter construct was co-transfected along with pRL-TK (internal control). Twenty four hours postinfection, cells were harvested with passive lysis buffer and luciferase activity was monitored using dual luciferase assay system (Promega, Madison, Wis.) following manufacturer's instructions. The PLA1A promoter fragment was PCR amplified using a genomic BAC clone as template with the forward primer (5′-CCCCATTGACTTGCCTAGAA (SEQ ID NO:1)) and reverse primer (5′-GGCTTTTAGGGGATCTTCCA (SEQ ID NO:2) and subcloned into pGL4.14 vector (Promega) using Xho1 and Hind3 enzymes.

### Quantitative Real-Time PCR Assays

Total RNA was isolated from VCAP, RWPE or PrEC cells that were transfected with siRNA as indicated (Qiagen). Quantitative PCR (QPCR) was performed using SYBR Green dye on an Applied Biosystems 7300 Real Time PCR system (Applied Biosystems, Foster City, Calif.) as described (Tomlins, S. A., et al. Science (New York, N.Y. 310, 644-648 (2005)). Briefly, 1 μg of total RNA was reverse transcribed into cDNA using SuperScript III (Invitrogen, Carlsbad, Calif.) in the presence of random primers (Invitrogen). All reactions were performed in triplicate with SYBR Green Master Mix (Applied Biosystems) plus 25 ng of both the forward and reverse primer according to the manufacturer's recommended thermocycling conditions, and then subjected to melt curve analysis. Threshold levels for each experiment were set during the exponential phase of the QPCR reaction using Sequence Detection Software version 1.2.2 (Applied Biosystems). The relative quantity of the target gene was completed for each sample using the ΔΔCt method by the comparing mean Ct of the gene to the average Ct of the housekeeping gene, β-Actin8. All oligonucleotide primers were synthesized by Integrated DNA Technologies (Coralville, Iowa). The primer sequences for the transcript analyzed were either previously described (Tomlins et al., 2008, supra) or are provided in Table 4.

### Gene Expression Array Analysis

Expression profiling was performed using the Agilent Whole Human Genome Oligo Microarray (Santa Clara, Calif.) according to the manufacturer's protocol and described previously (Tomlins, S. A., et al. Nature 448, 595-599 (2007)). For all hybridizations, the reference was VCaP treated with control siRNA for 48 hrs. A total of 4 arrays were performed per sample such that all hybridizations were performed in duplicate with duplicate dye flips. Data was filtered to include only features with significant differential expression (Log ratio, P<0.01) in all hybridizations and, after correction for the dye flip, two-fold average over- or under-expression (Log ratio). Over- and under-expressed signatures for the VCaP siRNA experiment were generated by identifying differential features common among both 2 independent DNA-PKcs siRNA sets and 2 independent PARP1 siRNA sets.

### Molecular Concept Maps

The expression signatures in common between both DNA-PKcs and PARP1 siRNA were uploaded into the Oncomine Concepts Map as molecular concepts (Rhodes, D. R., et al. Neoplasia (New York, N.Y. 9, 443-454 (2007)), using all features on the Agilent Whole Human Genome Oligo Microarray as the null set. Data are reported as RMA-normalized fluorescent intensities and analysis was completed as previously described (Tomlins et al., 2008, supra).

### Basement Membrane Matrix Invasion Assays

For invasion assays, the prostate cell lines RWPE-1 and VCaP were transfected with siRNA or negative controls as indicated. NU7026 (Sigma) and NU1025 (Calbiochem) were dissolved in DMSO and stored at −20° C. in the dark. Forty-eight hours post-transfection/transduction, cells were seeded onto the basement membrane matrix (EC matrix, Chemicon, Temecula, Calif.) in the chamber insert with 8.0 μM pores of a 24-well culture plate in serum free media. Cells were attracted to the lower chamber by the addition of complete media as a chemoattractant. After 48 hours incubation at 37° C. with 5% CO₂, the non-invading cells and EC matrix were gently removed with a cotton swab. Invasive cells, which were located on the lower side of the membrane, were stained with crystal violet, air dried and photographed. To quantify the relative number of invaded cells, colorimetric assays were performed by treating the inserts with 150 μl of 10% acetic acid (v/v) and measuring absorbance of each condition at 560 nm using a spectrophotometer (GE Healthcare).

### Preclinical Study Using Human Prostate Cancer Tissue Xenografts

Human prostate cancer specimens MDA-PCa 2b-T (ETV1 positive), MDA-PCa-133 (ERG positive) and MDA PCa 118b (ETS-negative) were obtained (Li, Z. G., et al. The Journal of clinical investigation 118, 2697-2710 (2008)). Each xenografted tumor tissue was aseptically harvested from the host mice. New recipient mice were anesthetized by injecting xylazine (80 mg/kg) and ketamine (10 mg/kg) intraperitoneally for chemical restraint before tumor implantation. About 0.5 cm³ size of the excised tumor tissue was directly implanted into subcutaneous pockets of 6- to 8-week-old male CB17 SCID mice (Charles River Laboratories) in a laminar flow sterile hood. Sub-cutaneous pocket was carefully closed by applying 1 or 2 wound clips. All mice were monitored for 24 hours after implantation procedure for any apparent physiologic disturbances or missing wound clips. Olaparib (40 mg/Kg body weight) treatment was started after 72 hours. Tumor growth was recorded twice a week using digital calipers and tumor volumes were calculated using the formula (*/6) (L×W²), where L=length of tumor and W=width. Body weight was also monitored weekly during the course of the study. All procedures involving mice were approved by the University Committee on Use and Care of Animals (UCUCA) at the University of Michigan and conform to their relevant regulatory standards.

### COMET Assay

VCaP, PC3, PrEC or RWPE cells were seeded at 250,000 cells/mL in a 6-well plate 24 hours prior to treatment with siRNA, drug or vehicle control. After 48 hours, cells were trypsinized, harvest by centrifugation and re-suspend in PBS. Cell counts were then normalized to 1×10⁵ cells/mL. Suspended cells (25 μL) were then mixed with 250 μL 1.0% ultrapure low melting point agarose (Invitrogen) made in 1× Tris-Borate buffer. The agarose-cell mixture was then dropped onto slides allowed to solidify at 4° C. in the dark for 20 minutes before immersion in COMET assay lysis solution (Trevigen, Gaithersburg, Md.) at 4° in the dark for 45 minutes. Excess buffer was then removed and slides were submerged in freshly prepared neutral solution (Tris Base 60.57 g, Sodium Acetate 204.12 g, dissolve in 450 ml of dH₂O, adjust to pH=9.0 with glacial acetic acid) at room temperature in the dark for 40 minutes. Slides were then washed twice by immersing in 1×TBE buffer prior to neutral electrophoresis at 20 volts for 30 minutes. Slides were then fixed in 70% ethanol for 5 minutes. Following air drying of the agarose, slides were stained with SYBR Green dye (Invitrogen) and images were collected with a 10× and 40× objective lens. COMET tail moments were then assessed using COMETscore.v1.5 (AutoCOMET.com, Sumerduck, Va.) image processing software as described by the manufacturer with greater than 100 cells analyzed in triplicate experiments.

Expression and confirmation of HaloTag fusion proteins. ETS genes (as described), ERG and sub-domains were cloned into pFN19A vector (Promega, Wisconsin) according to the manufacturer's instructions. Alanine scan mutations were created in the HALO-ETS pFN19A expression vector according to standard Quikchange XL site directed mutagenesis kit protocol (Stratagene). Fusion proteins were expressed in TNT® SP6 High-Yield Wheat Germ Reaction (Promega) based on the manufacturer's protocol. A total of 2.0 μl of cell-free reaction containing the HaloTag® fusion protein was mixed with 8 μl HaloTag® Biotin Ligand (final concentration 1 μM), and incubated at room temperature for 30 minutes. The biotin-labeled samples were separated on SDS gel and blotted using HRP-streptavidin.
Interaction assays. A total of 100 μl of cell-free reaction containing ETS proteins or ERG and ERG sub-domain fusion proteins were incubated with 224 U of DNA-PKcs (Promega) or PARP1 (Trevigen) in PBS-T (0.1% tween) overnight at 4° C. Ten microliters of HaloLink beads (Promega) were then blocked in BSA overnight at 4° C. After incubation the beads were washed 3 times with PBS, the beads were mixed with Halo-ERG:DNA-PKcs mixture and incubated at RT for 1 hr. Halolink beads were then washed with PBST for 4 times and eluted in SDS loading buffer. Proteins were separated on SDS gel and blotted with anti-DNA PKcs Ab (Santa Cruz). Halo-GUS fusion proteins were used as negative controls.
Homologous Recombination Assays. Stable PC3 cell lines were transfected with HR-reporters (Norgen Biotek Corp., Thorold, ON) using Fugene 6.0HD (Roche) according to the manufacturer's protocol. Briefly, total DNA was isolated from cells 24 hours after transfections using the DNeasy blood and tissue DNA extraction kit according to manufacturer protocol (Qiagen). qPCR was then performed to assess HR-efficiency using primer sets to specifically detect recombination (Norgen Biotek Corp).

### B. Results

### Characterization of ERG Interacting Proteins

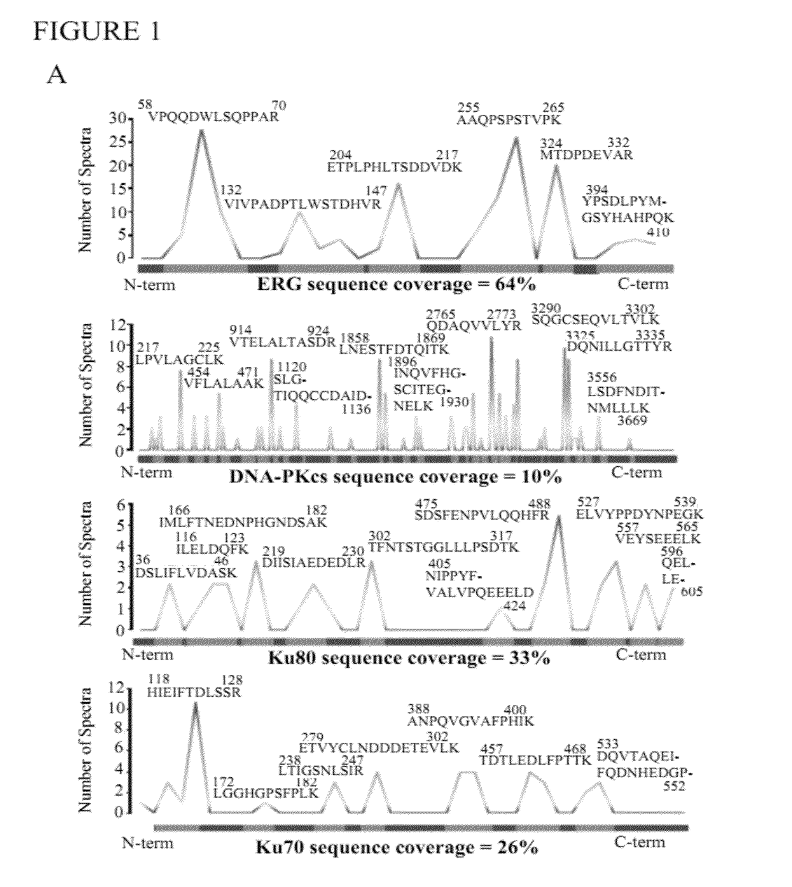

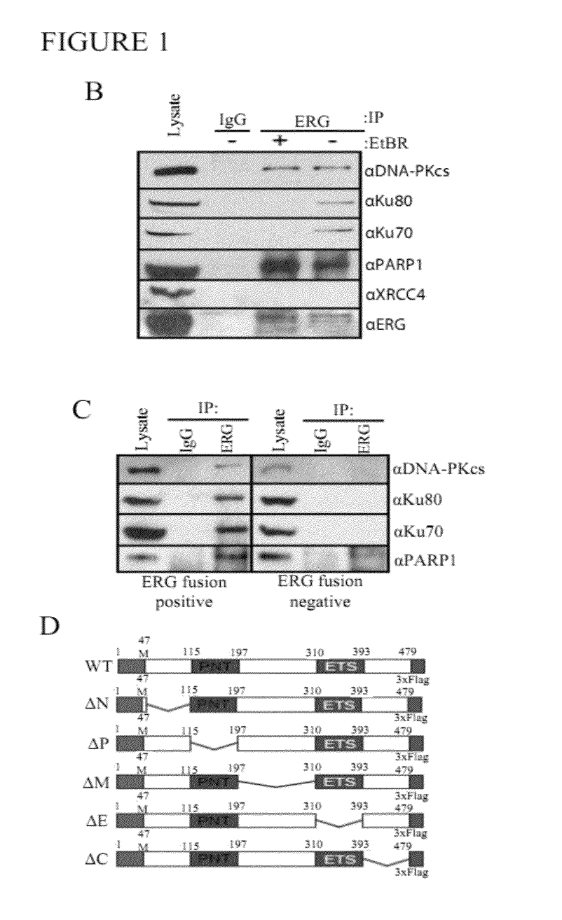

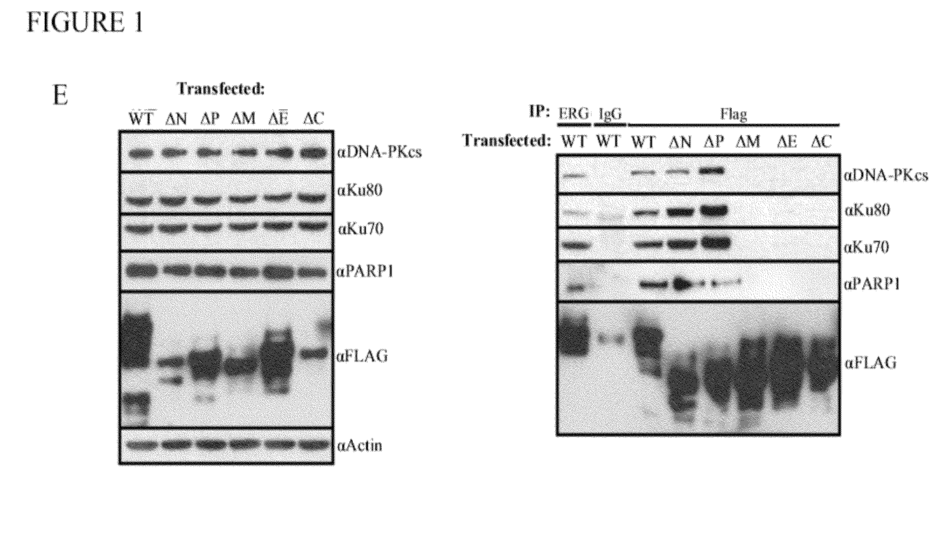

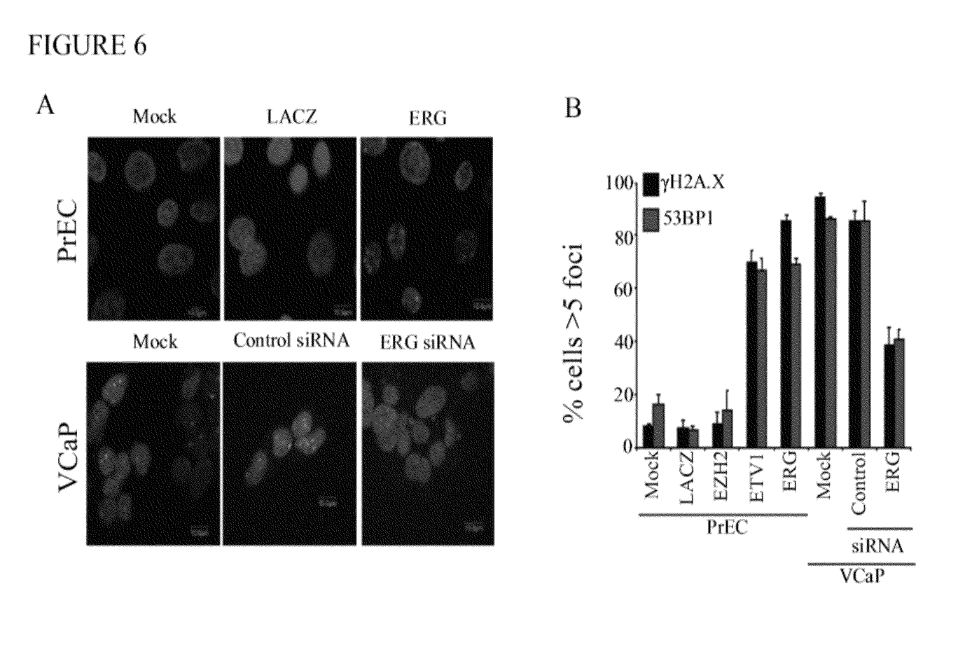

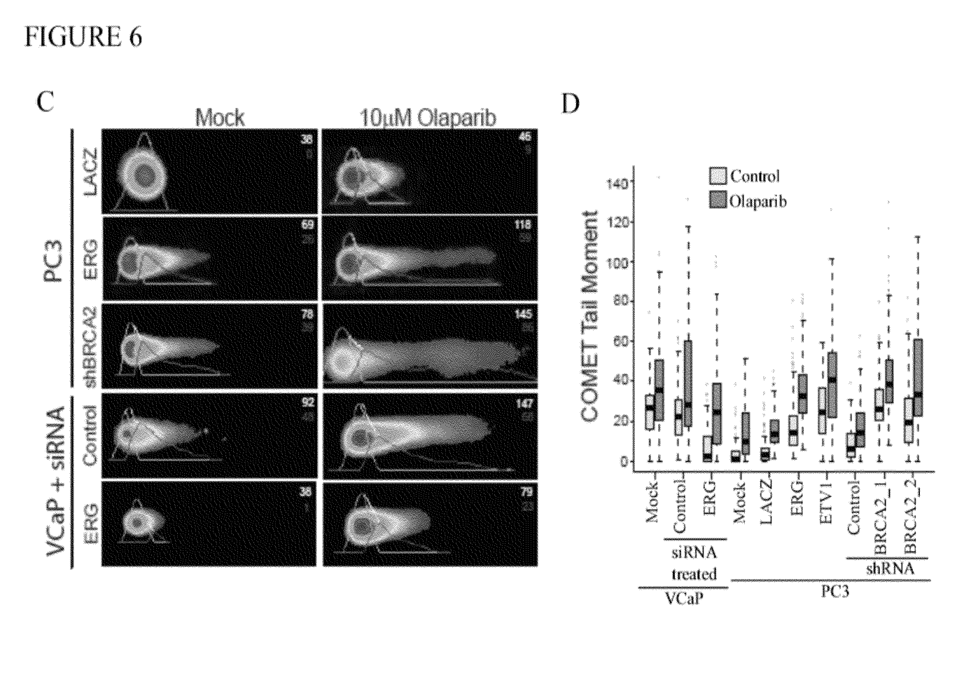

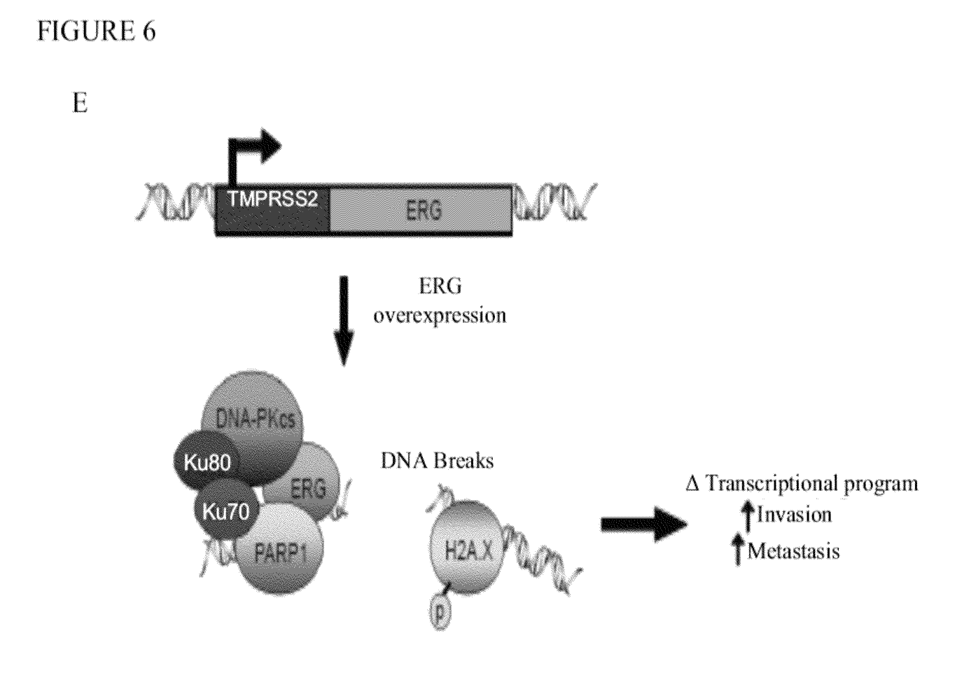

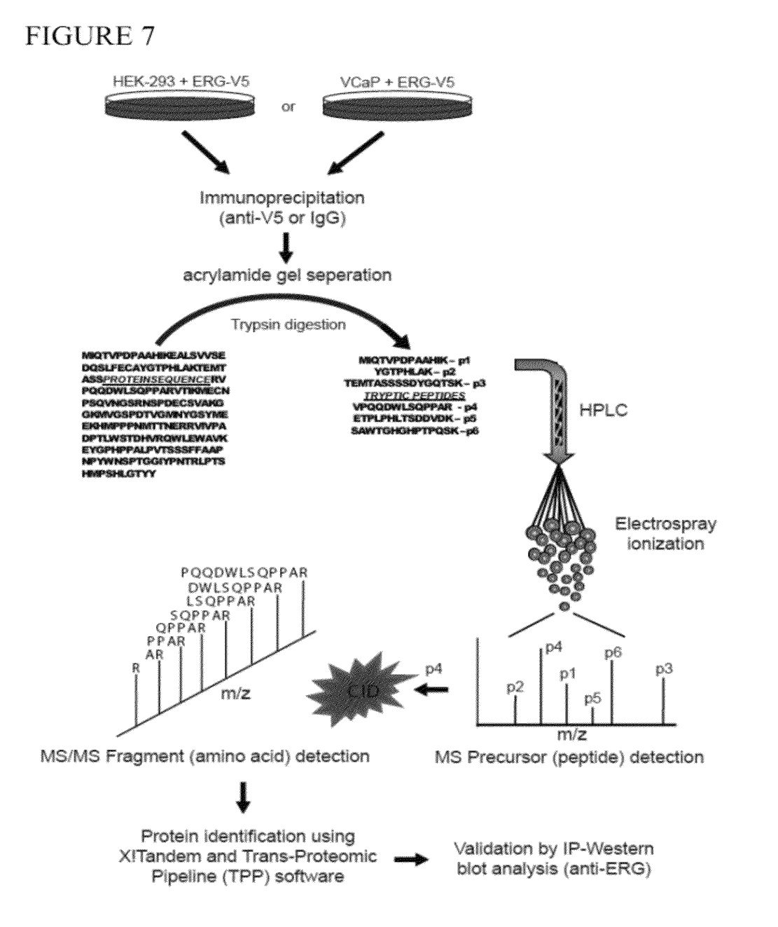

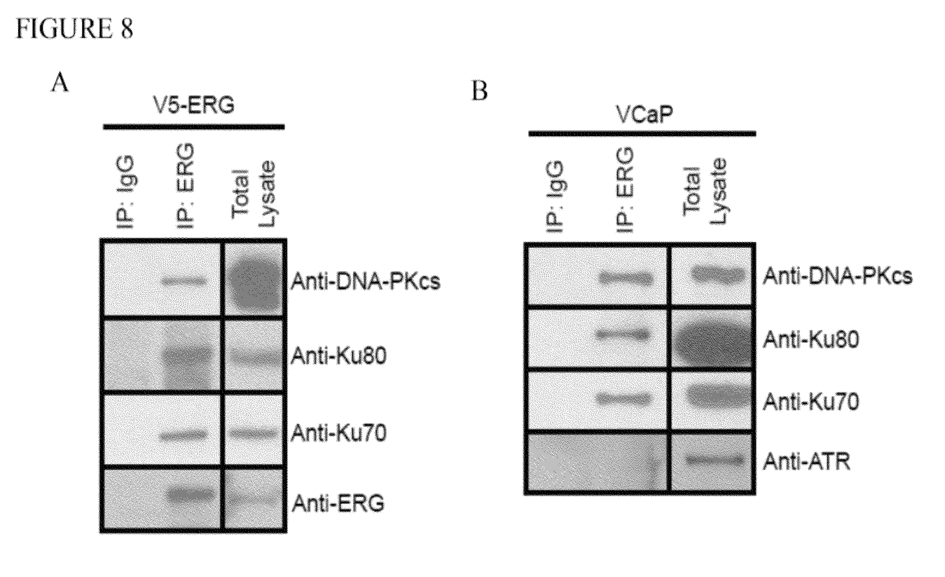

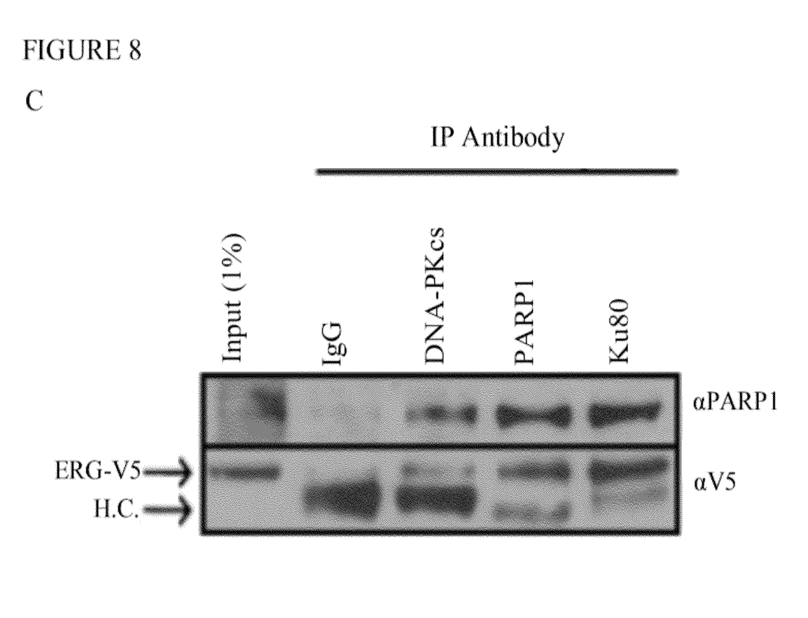

To identify ERG-interacting proteins that may serve as rational therapeutic targets and to shed light on the mechanism by which ETS gene fusions mediate their effects, mass spectrometric analysis of proteins that interact with the ETS gene fusion product, ERG was performed. Epitope-tagged expression vectors for the coding sequence of the most prevalent gene fusion product (TMPRSS2 exon 1 fused to ERG1 exon 2) were generated (Tomlins et al. Science (New York, N.Y. 310, 644-648 (2005)). VCaP prostate cancer cells (which harbor the TMPRSS2:ERG rearrangement) or human embryonic kidney cells (HEK) 293 cells were infected with either adenoviral V5- or FLAG-epitope-tagged ERG expression vectors, respectively. Immunoprecipitation with each tag was completed in 8 biological replicates to isolate protein-protein interactions with ERG. The collected immunoprecipitate was analyzed by liquid chromatography-tandem mass spectrometry to identify putative protein-protein interactions (described by schematic in FIG. 6). As a control, epitope-tagged beta-galactosidase (LACZ) was run in parallel and immunoprecipitated in each replicate experiment. Non-specific interactions identified in the vector controls were manually removed from the experimental protein listing to generate a final list of genuine ERG interactions found in both HEK293 and VCaP cells over eight biological replicates. The interaction bait, ERG, was the top scoring protein identified in the pull-down with 64.4% coverage and 573 total peptides detected (FIG. 1a). Three of the next 4 interacting proteins of high confidence and high sequence coverage identified were components of the DNA-dependent protein kinase complex and included the large catalytic subunit of a phosphatidylinositol 3/4 (PI3/4)-kinase called DNA-dependent protein kinase (DNA-PKcs) (10% coverage), and its known interacting subunits Ku70 (26% coverage) and Ku80 (34% coverage) (FIG. 1a). A list of protein interactions identified with high confidence in these experiments is provided in Table 1. To confirm the interaction between ERG and the DNAPK complex (which consists of DNA-PKcs:Ku70:Ku80), V5-ERG was overexpressed in VCaP cells and, following immunoprecipitation with a V5 antibody, a strong interaction between ERG and DNA-PKcs, Ku70 and Ku80 was observed (FIG. 8a). To then determine if this interaction occurs endogenously in VCaP prostate cancer cells, ERG was immunoprecipitated in the absence of ectopic overexpression and a similar association with DNA-PKcs, Ku70 and Ku80 was observed (FIG. 1b and FIG. 8b).

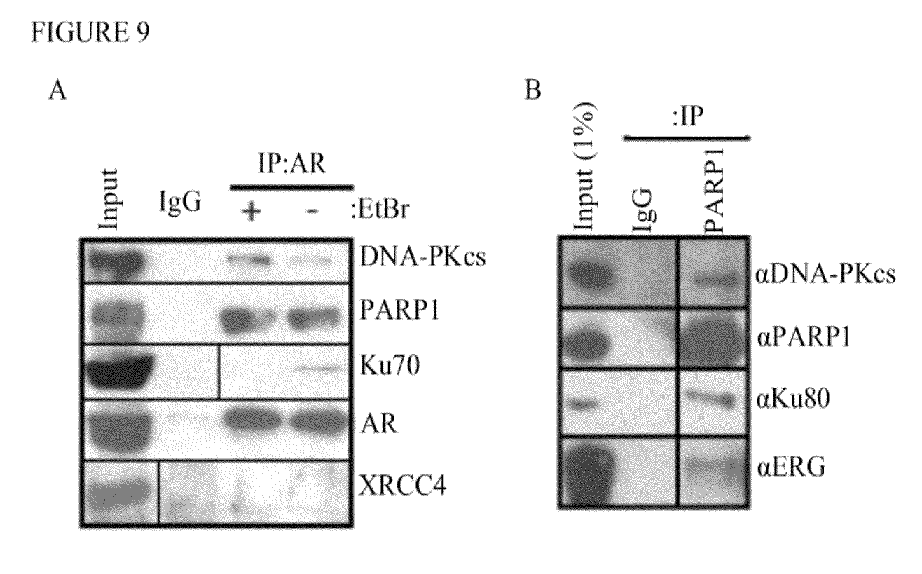

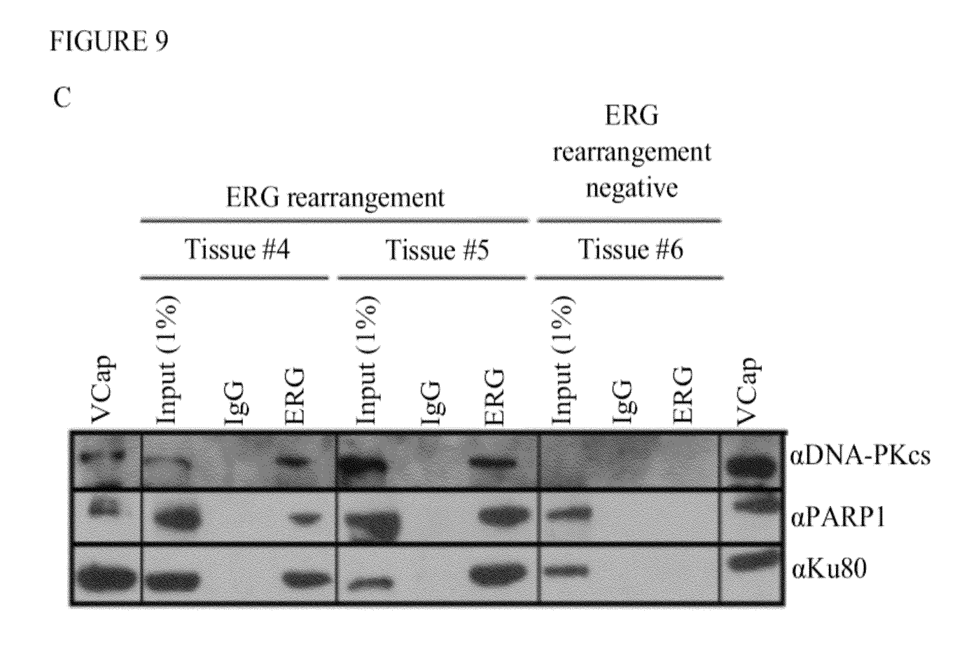

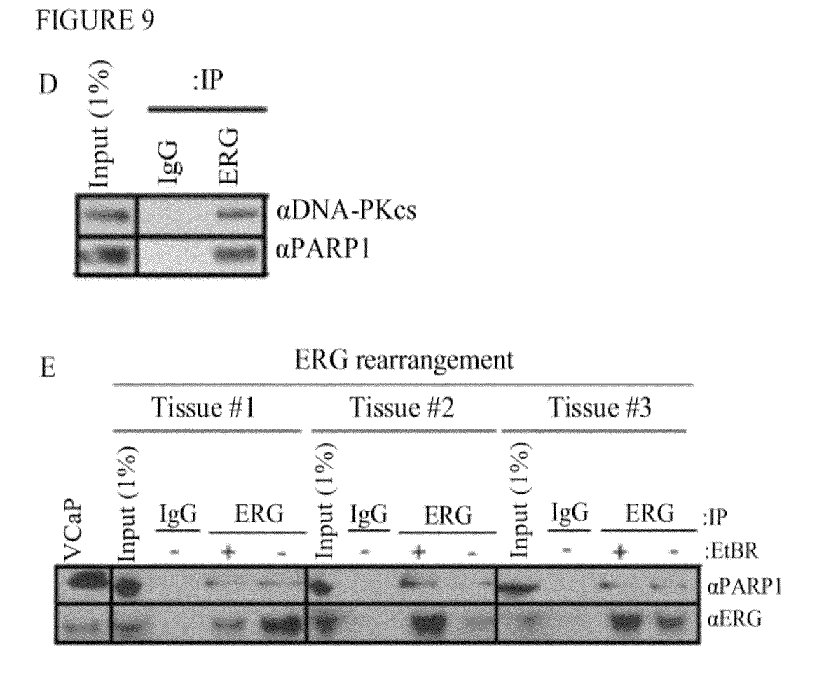

To identify additional proteins participating in the ERG:DNAPK complex, the list of ERG interactors was assessed for other proteins known to interact with DNA-PKcs, Ku70 or Ku80 and two peptides for poly-(ADP-ribose) polymerase (PARP1) were identified: VVSEDFLQDVSASTK (SEQ ID NO:3) and QQVPSGESAILDR (SEQ ID NO:4). PARP1 endogenously associated with ERG in VCaP cells (FIG. 1b). Reverse IPs using antibodies against DNA-PKcs, PARP1, and Ku80 and was performed. It showed that each antibody was able to detect ERG-V5 protein (FIG. 8c). To detect the PARP1:ERG interaction with the endogenous TMPRSS2:ERG gene fusion product, agarose-coupled PARP1 antibody was used to perform an IP-western, which confirmed that PARP1 interacts with the gene fusion product in an endogenous setting (FIG. 9B).

Because DNA-PKcs only binds with Ku70 and Ku80 in the presence of DNA (Spagnolo et al., Molecular cell 22, 511-519 (2006)), the dependence of the ERG:PARP1:DNA-PKcs interaction on intact DNA was tested by performing the immunoprecipitation in the presence of 100 μM ethidium bromide. This treatment disrupted the interaction between ERG and Ku70 and Ku80, but not the interaction between ERG and PARP1 or DNA-PKcs, demonstrating that the ERG:PARP1:DNA-PKcs interaction is DNA-independent (FIG. 1b). As a control, it was tested whether ERG would bind another PI3/4 kinase family member, ATR, or another protein known to interact with the DNAPK complex, XRCC4. The latter protein is required for DNA-PKcs-mediated repair of DNA double strand breaks through direct interactions with Ku70 and Ku80 during a process called non-homologous end joining (NHEJ) (Nick McElhinny et al., Molecular and cellular biology 20, 2996-3003 (2000); Chen et al., The Journal of biological chemistry 275, 26196-26205 (2000)). Consistent with the immunoprecipitation-mass spectrometry data, no interaction between ATR or XRCC4 and ERG was detected by immunoprecipitation-Western blot analysis (FIG. 8b and FIG. 1b, respectively). This indicates that the ERG complex associates with DNA-PKcs through specific interactions not shared with other PI3/4 kinases and that the ERG complex is not directly associated with the DNA-PKcs repair pathway. As it has previously been demonstrated that ERG interacts with androgen receptor (AR) (Yu, J., et al. Cancer cell 17, 443-454), IP-western blot using an anti-AR antibody was performed as an additional positive control (FIG. 9a). This demonstrated that AR interacts with the DNA-PKcs:PARP1 complex in VCaP cells.

After confirming the ERG:PARP1:DNA-PKcs interaction in prostate cancer cell lines, it was further assessed whether the protein interactions occur endogenously in human prostate cancer tissues. Immunoprecipitation experiments from ERG-gene fusion positive and negative prostate cancer tissue samples were performed. Immunoprecipitation with an ERG antibody was sufficient to pull down DNA-PKcs, Ku70, Ku80 and PARP1 in the ERG gene fusion prostate cancer tissues, but not in the ERG gene fusion-negative prostate cancer tissues (FIG. 1c). This indicated that either the interaction does not occur in the rearrangement-negative tissue or that the interaction occurs, but is undetectable by IP-Western because of the low level expression of ERG protein in the control sample. Additional IP-westerns were performed to test the dependence of the ERG:PARP1 interaction on DNA in human prostate cancer tissues. The interaction occurred in the absence of DNA in all three independent human tissues (FIG. 9E).

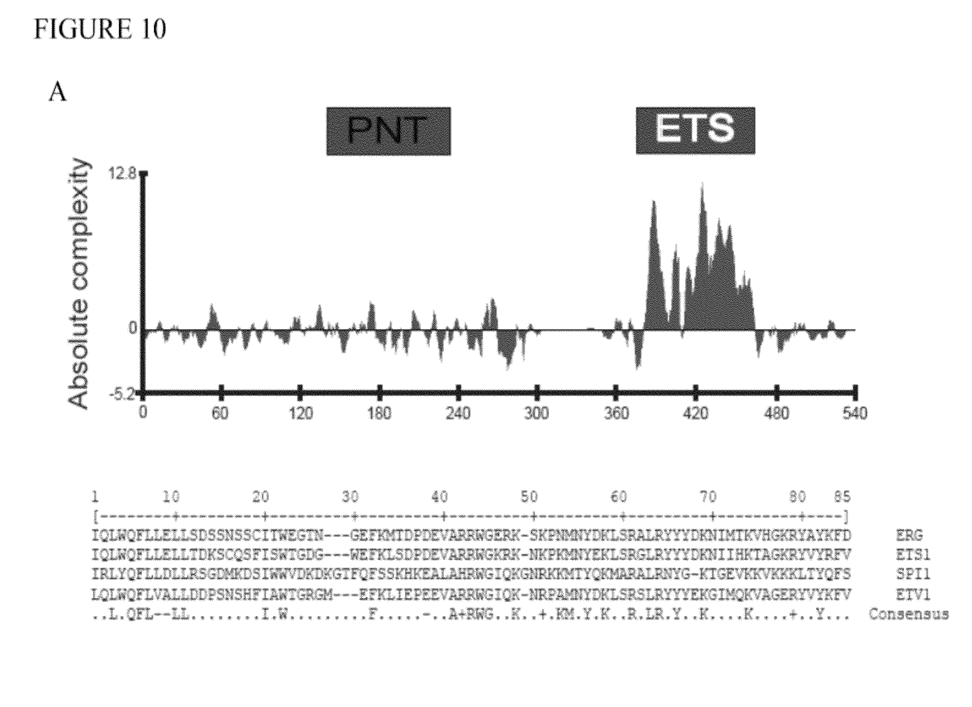

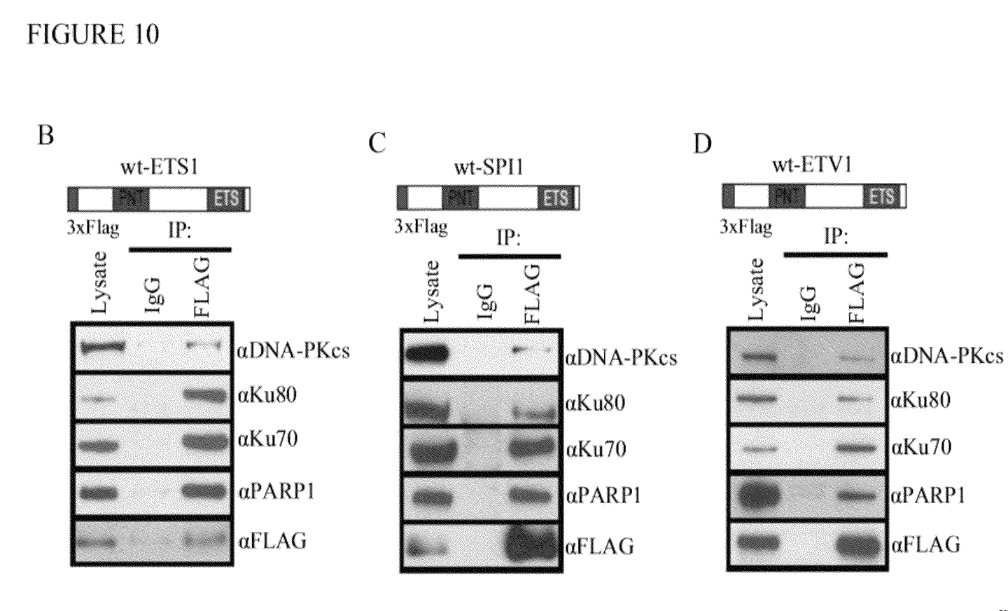

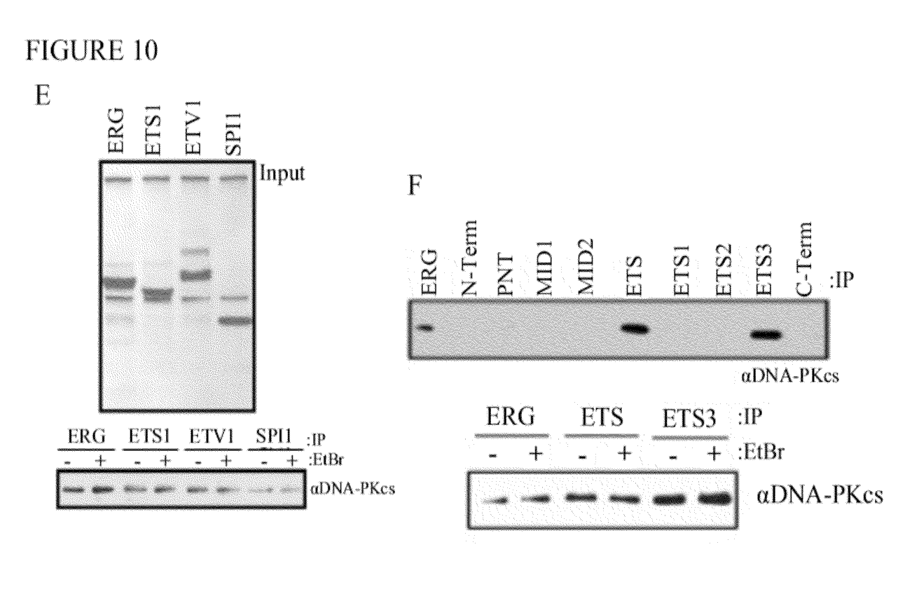

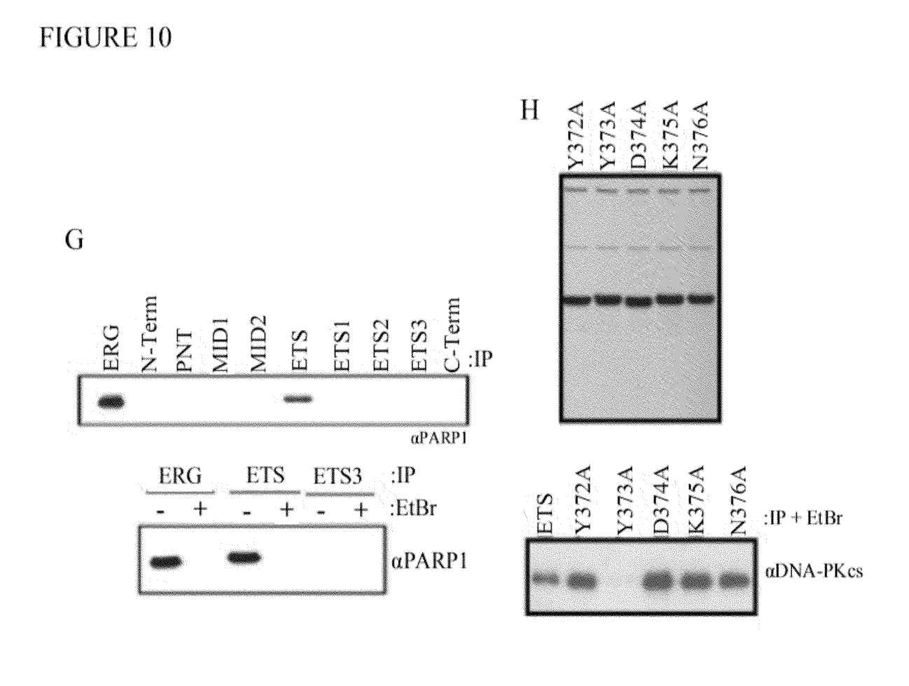

Next, a series of expression vectors of the most prevalent gene fusion product (TMPRSS2 exon 1 to ERG1 exon 2) were generated by deleting either the N-terminus (AA: 47-115), pointed domain (AA: 115-197), the middle amino acids (197-310), the ETS domain (AA: 310-393) or the C-terminus (AA: 393-479). The constructs were labeled ΔN, ΔP, ΔM, ΔE, ΔC, respectively. Three FLAG antigen sequences were fused to the c-terminus of each gene for immunoprecipitation as depicted in FIG. 1d. The predicted molecular weight of the full length protein encoded from the longest open reading frame of these constructs is ΔN=44.6 kDa, ΔP=43.4 kDa, ΔM=41.6 kDa, ΔE=43.7 kDa, ΔC=43.6 kDa. Transient transfection of these constructs into HEK293 cells demonstrated that the interactions between ERG, DNA-PKcs, Ku70, Ku80 and PARP1 occurred in the c-terminal half of the ERG protein which contains the conserved ETS domain (FIG. 1e). Because these large tiling deletions may have disrupted the tertiary structure of the protein, it is difficult to determine the precise location of the interaction. The region of ERG shown to interact with DNA-PKcs and PARP1 is retained in all of the ERG gene fusions found in prostate cancer and represents an interaction critical for ERG-induced phenotypes (reviewed in Brenner, J. C. & Chinnaiyan, A. M. Biochimica et biophysica acta (2009)). To further map the ERG:PARP1:DNA-PKcs interaction and to confirm that PARP1 and DNA-PKcs both interact with other ETS family member proteins, immunoprecipitation-Western blot analysis was performed in HEK293 cells transfected with either ETS1-FLAG, SPI1-FLAG or ETV1-FLAG expression vectors. In all three experiments, pull downs confirmed the interactions (FIG. 10). N-terminal halotagged expression vectors were generated for in vitro purification of these ETS genes. Subsequent IP-westerns demonstrated that all four of these proteins bind directly to DNA-PKcs (FIG. 10E). Given the sequence alignment of these four ETS proteins and the large tiling deletion data, the data indicated that the interactions occur through the ETS DNA-binding domain. Furthermore, sequence alignment of ERG, ETS1, SPI1 and ETV1 demonstrated that the interaction occurs in the highly conserved ETS domain.

To map the ERG:DNA-PKcs and ERG:PARP1 interactions, HALO-tagged WT ERG and six individual ALO-tagged fragments spanning the entire ERG protein (Yu et al., Cancer Cell 17, 443-454 2010) were utilized. IP-western blot demonstrated that the ERG:DNA-PKcs interaction occurred through the ETS DNA-binding domain. To further map the interaction between ERG and DNA-PKcs, a series of three HALO-tagged fragments that tiled the ETS domain, which localized the interaction to the final 28 amino acids of the ETS domain (FIG. 1F; FIG. 10F) were utilized. Although the crystal structure of the ETS domain from ERG has not yet been reported, the crystal structure of another ETS factor that interacts with DNA-PKcs, ETS1, has been published (Garvie et al., Mol. Cell 8, 1267-1276 2001). Based on homology with other interacting ETS proteins and structural information, it was predicted that the interaction was dependent on the amino acids, YYDKN. By site-directed mutagenesis of each residue to alanine, it was demonstrated that the Y373A mutant was unable to precipitate DNAPKcs, indicating that this interaction is mediated by Tyrosine 373 (FIG. 1F; FIG. 10H). Analysis of the ETS1 structure shows that Y373 is adjacent to the arginine residues that fit into the DNA groove and that Y373 is accessible to potential interacting proteins (FIG. 1G). After demonstrating that ERG interacts with DNA-PKcs directly through amino acid Y373, the ERG:PARP1 interaction was mapped. However, purified ERG was only able to interact with purified PARP1 in the absence of ethidium bromide (FIG. 10G). Because the interaction occurred in cells independent of ethidium bromide, this indicates that the ERG:PARP1 interaction is mediated by other proteins. This is consistent with the results from the IP-MS experiment in which few PARP1 peptides were identified, indicating that the ERG:PARP1 interaction is mediated by an intermediate protein such as DNA-PKcs.

### PARP1 and DNA-PKcs are Required for ERG-Mediated Transcription and Invasion

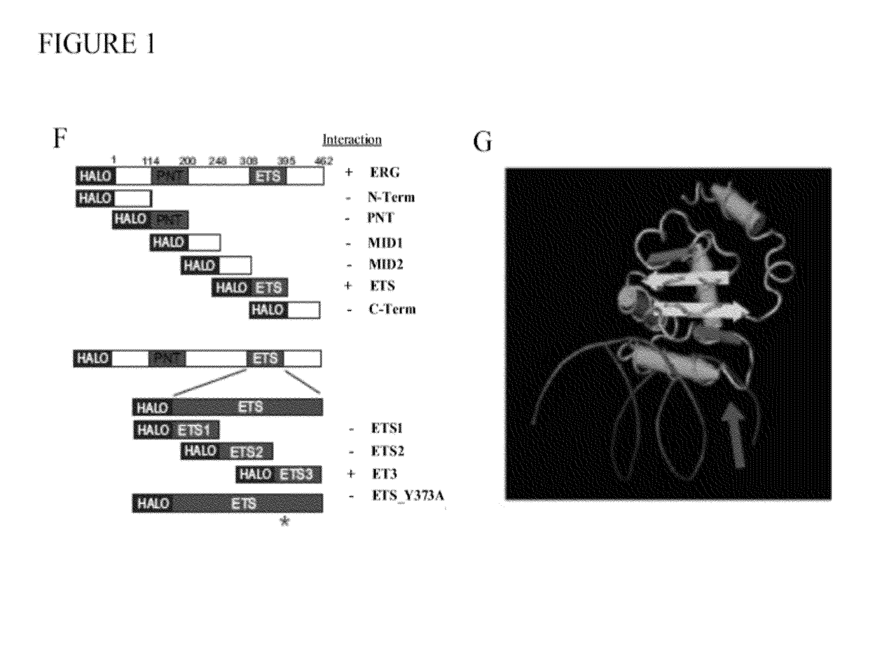

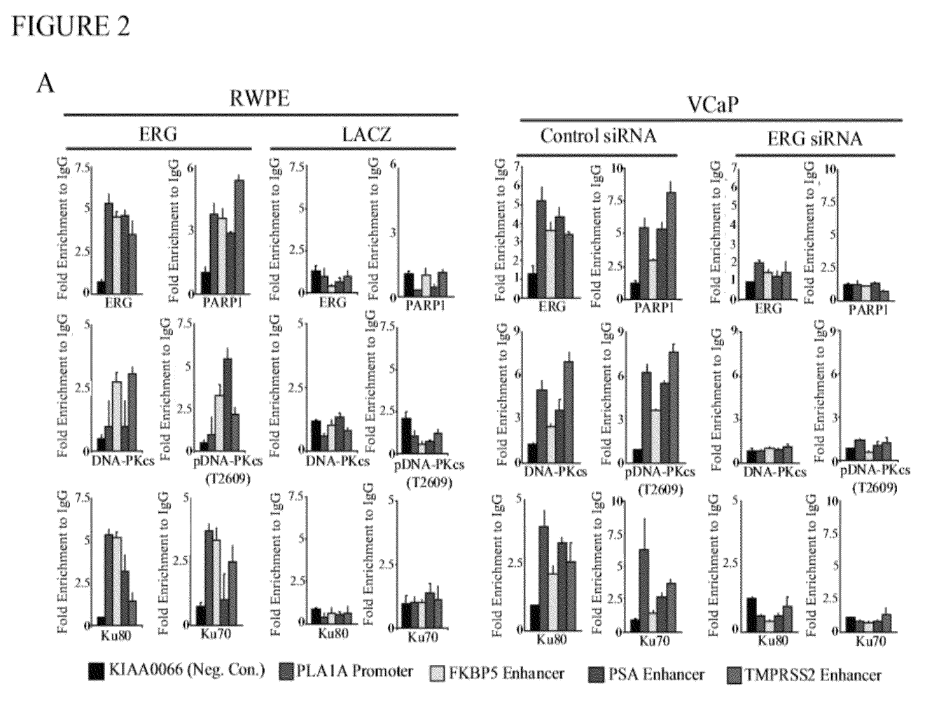

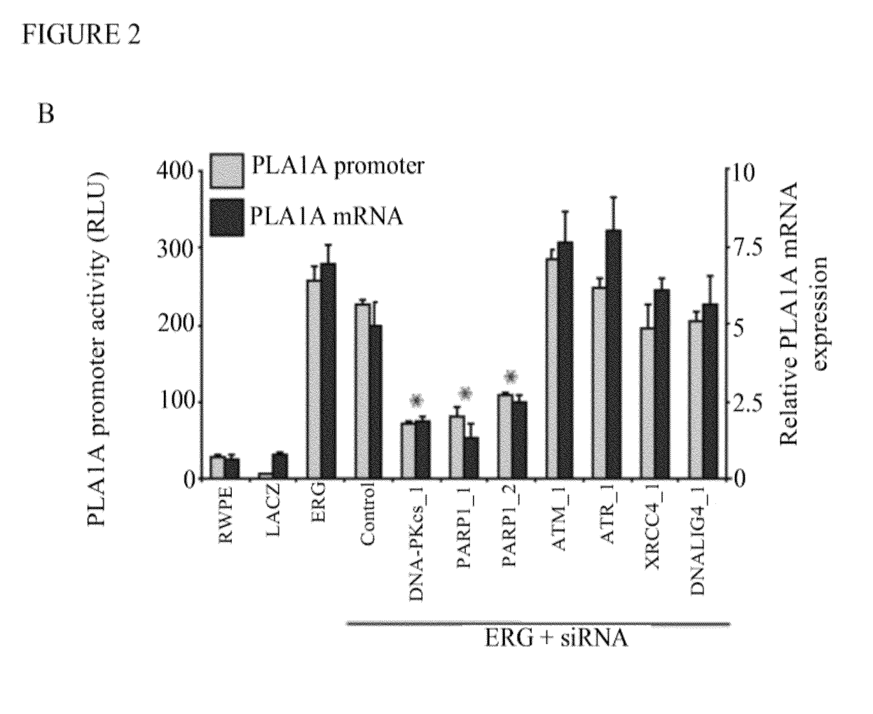

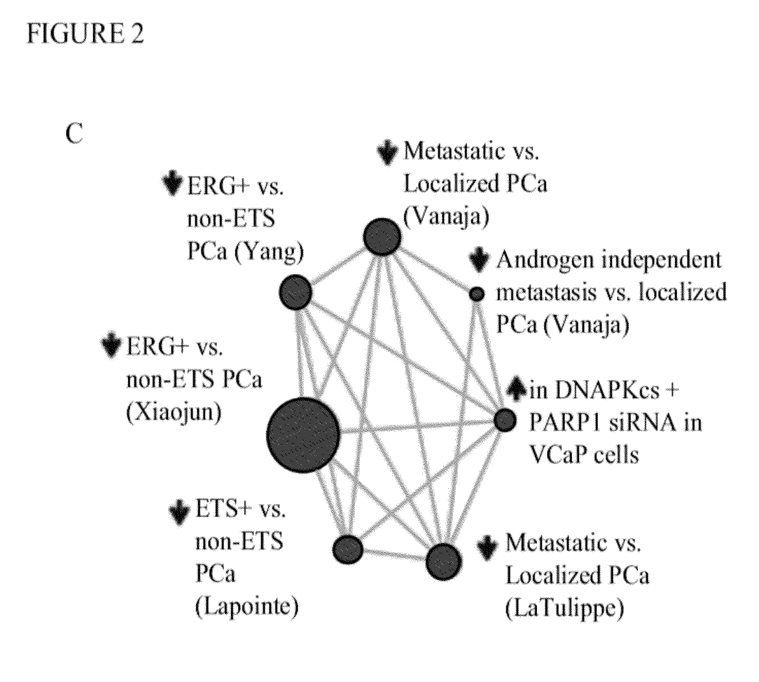

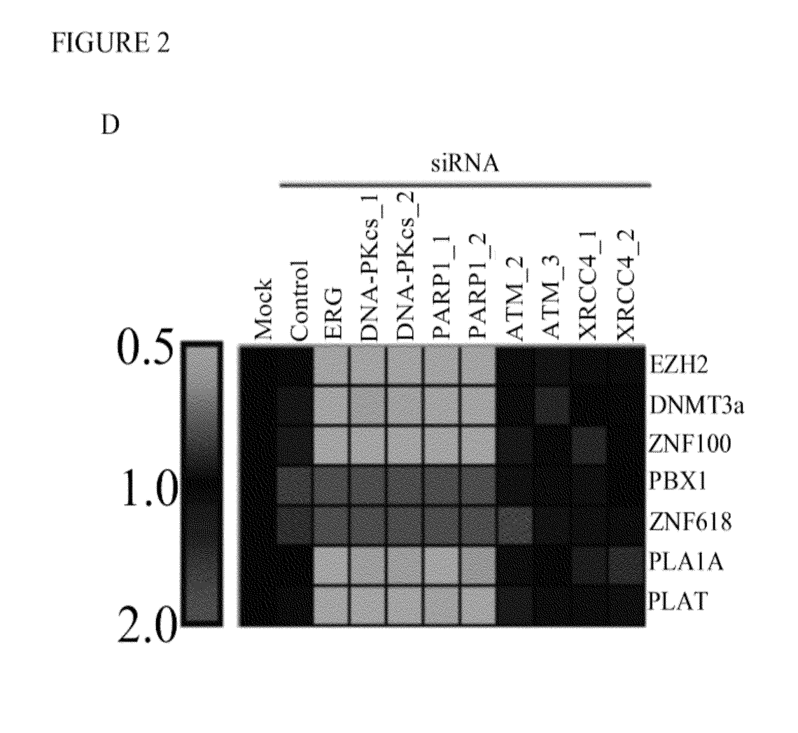

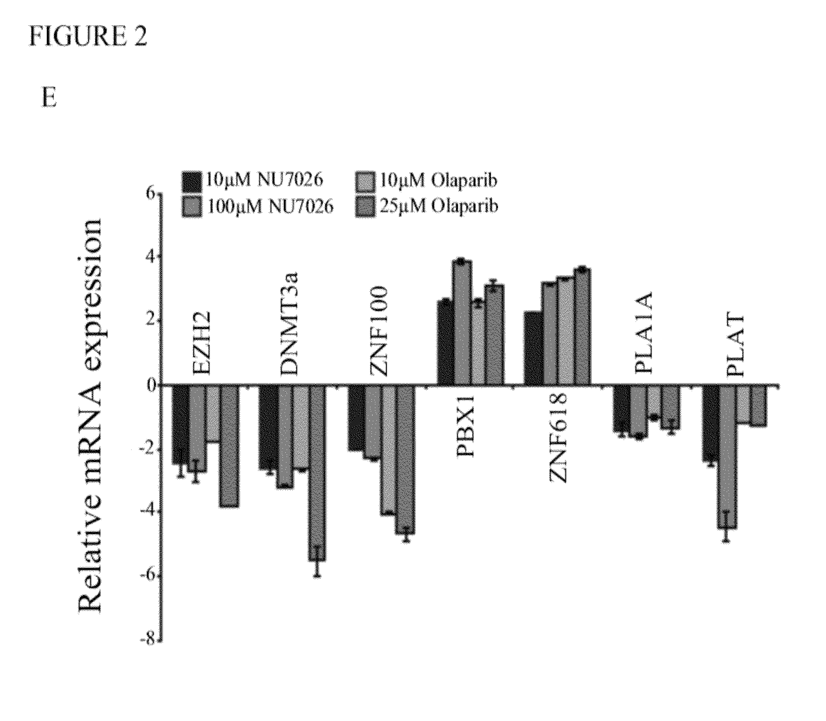

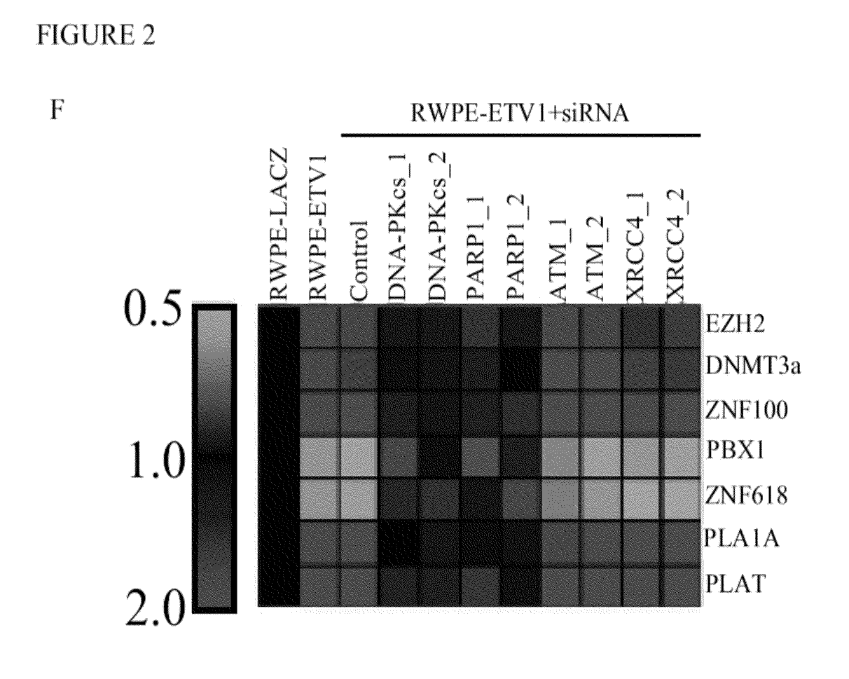

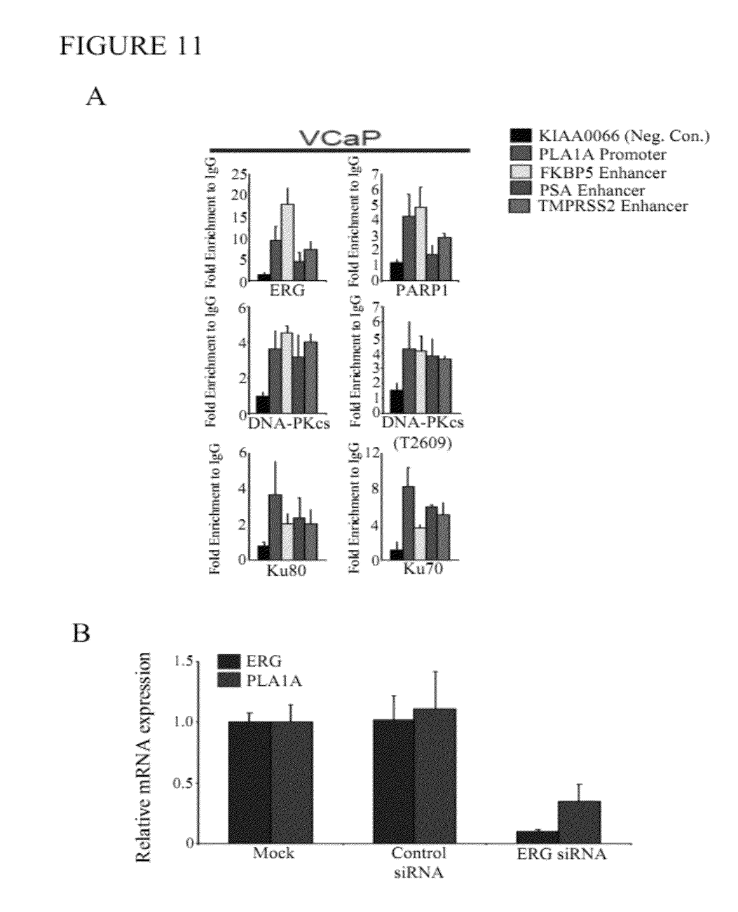

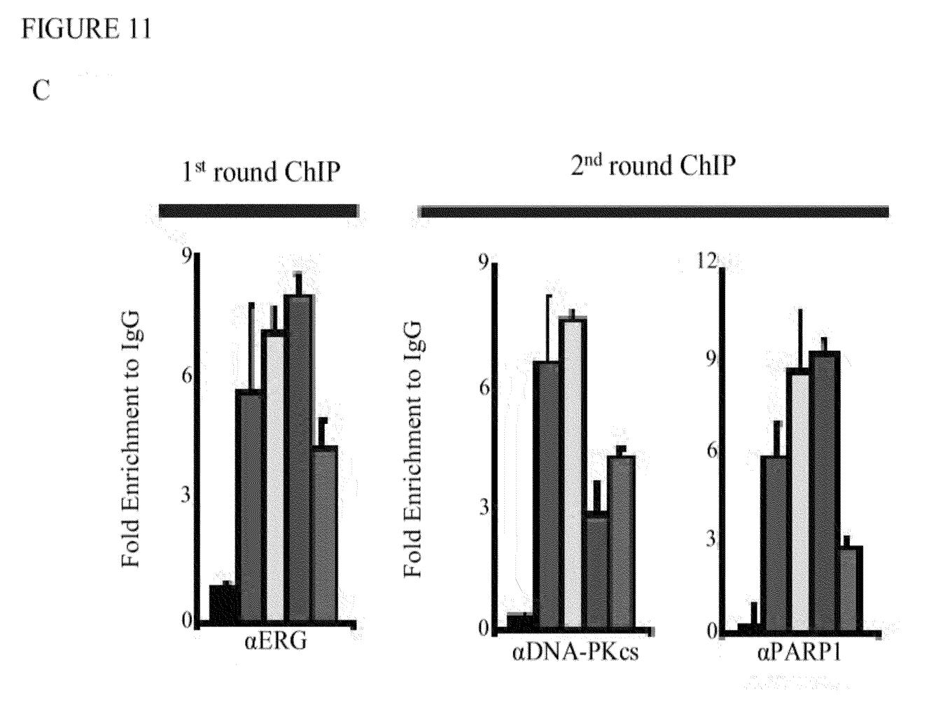

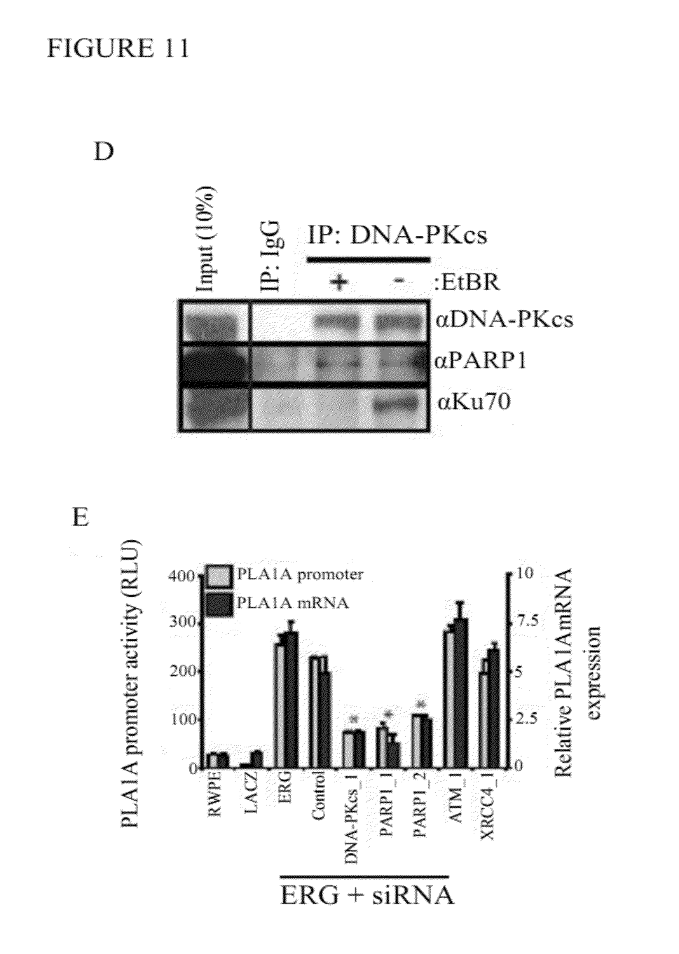

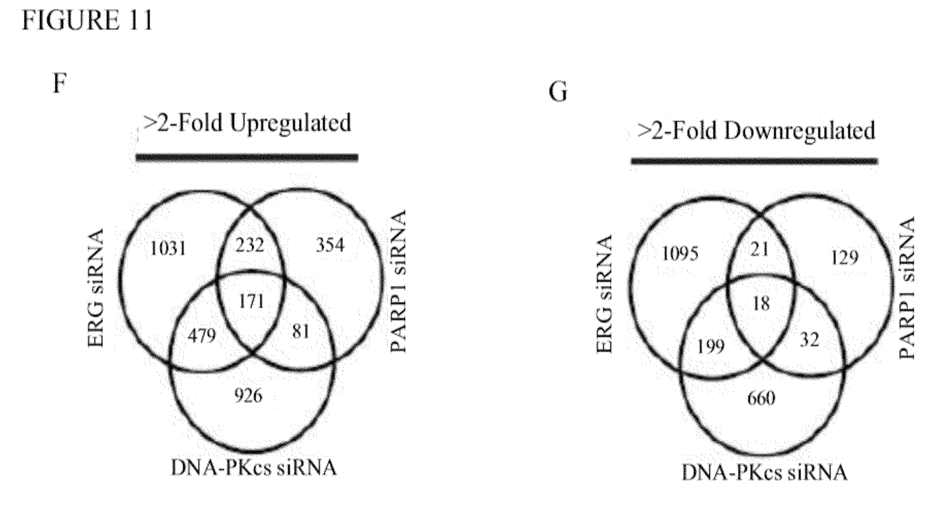

After confirming and mapping the ETS:PARP1:DNA-PKcs interaction, it was contemplated that both PARP1 and DNA-PKcs function as critical co-regulators of ERG transcriptional activity. Chromatin immunoprecipitation (ChIP) assays were performed in VCaP cells and enrichment of several ERG-targets including the PLA1A promoter and the FKBP5, PSA and TMPRSS2 enhancers was assessed. These experiments demonstrated that ERG, DNA-PKcs, Ku70, Ku80 and PARP1 were binding to these sites, but not to the negative control gene KIAA0066 (Brenner and Chinnaiyan, supra) (FIG. 11a). Previously, phosphorylation of DNA-PKcs on T2609 has been shown to be critical for DNA-PKcs activation (Chan et al. Genes & development 16, 2333-2338 (2002)). As such, ChIP was used to determine the T2609 phosphorylation state of the DNA-PKcs bound to ERG-regulated loci. These experiments found enrichment for all four of the ERG-regulated genes tested, but not the negative control (FIG. 11a). After confirming that PARP1 and activated DNA-PKcs were present at ERG-regulated genomic loci, it was tested whether ERG overexpression recruits DNA-PKcs and PARP1 to ERG-regulated DNA. ChIP performed with antibodies against ERG, PARP1, p-DNA-PKcs, DNA-PKcs, Ku70 and Ku80 were all able to enrich several ERG-regulated promoters and enhancers, but not the control KIAA0066 relative to IgG controls, in an ERG-overexpressing, but not a control cell line (FIG. 2a). Similarly, treatment of VCaP cells with ERG siRNA blocked the ability of PARP1, p-DNA-PKcs, DNA-PKcs, Ku70 and Ku80 antibodies to enrich the ERG-regulated gene promoters and enhancers (FIG. 2a and FIG. 11b). This data indicates a model in which ERG recruits the PARP1:DNA-PKcs complex to specific genomic loci during transcription.

To then test whether DNA-PKcs and PARP1 are required for ERG-mediated transcriptional activation, a PLA1A promoter reporter was constructed. Transfection of the reporter into RWPE cells treated with either LACZ or ERG adenovirus and siRNA (FIG. 12) indicated that both DNA-PKcs (P=1.99×10⁻³) and PARP1 (P=2.37×10⁻³) are required for ERG-induced activation of PLA1A (FIG. 2b) in RWPE cells. Inhibition of the related PI3/4-like kinases, ATM or ATR, had no significant effect on ERG activity.

While ATM and ATR repair DNA strand breaks through different pathways, DNA-PKcs is specifically required for NHEJ (Weterings, & Chen, The Journal of cell biology 179, 183-186 (2007)). In this process, DNA-PKcs, Ku70 and Ku80 form a complex on the broken DNA end which facilitates DNA end processing and re-joining in a multi-step procedure that requires the XRCC4/DNA Ligase IV complex. XRCC4 and DNA Ligase IV are both independently required for execution of NHEJ in mammalian cells, as targeted inactivation of either gene leads to NHEJ defects in mouse cells (Barnes et al., Curr Biol 8, 1395-1398 (1998); Frank et al. Nature 396, 173-177 (1998). siRNA was used to knockdown either XRCC4 or DNA Ligase IV (FIG. 12) to evaluate if ERG-induced transcriptional activation of the PLA1A promoter required effective execution of NHEJ. Because knockdown of either XRCC4 or DNA Ligase IV had no effect on ERG activity, the experiment further indicates a NHEJ-independent role of DNA-PKcs in ERG-mediated transcription (FIG. 2b.

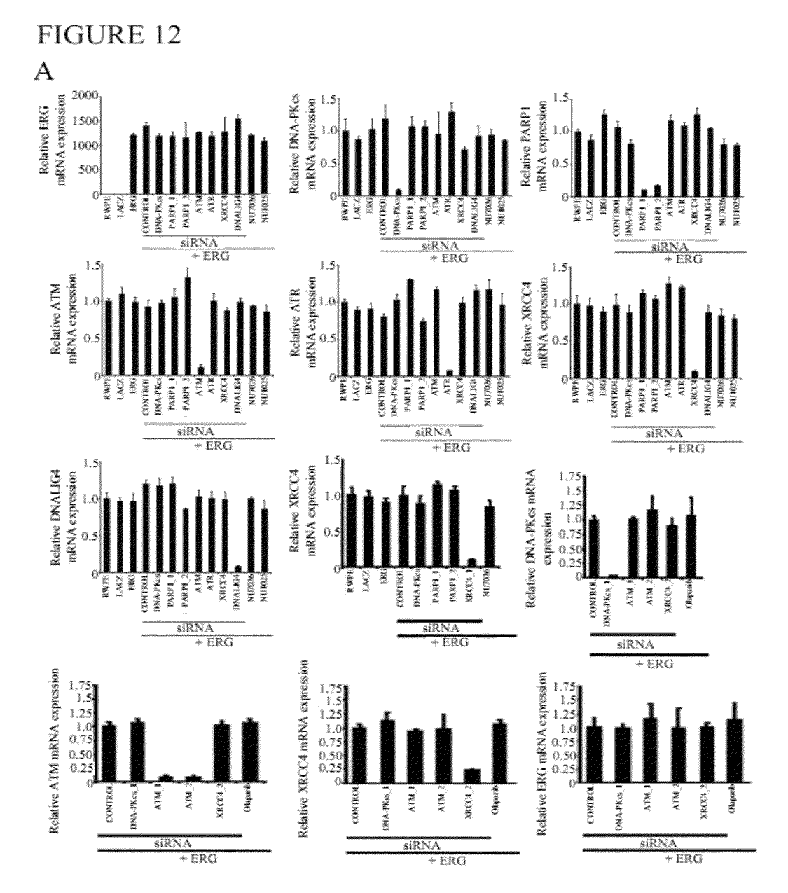

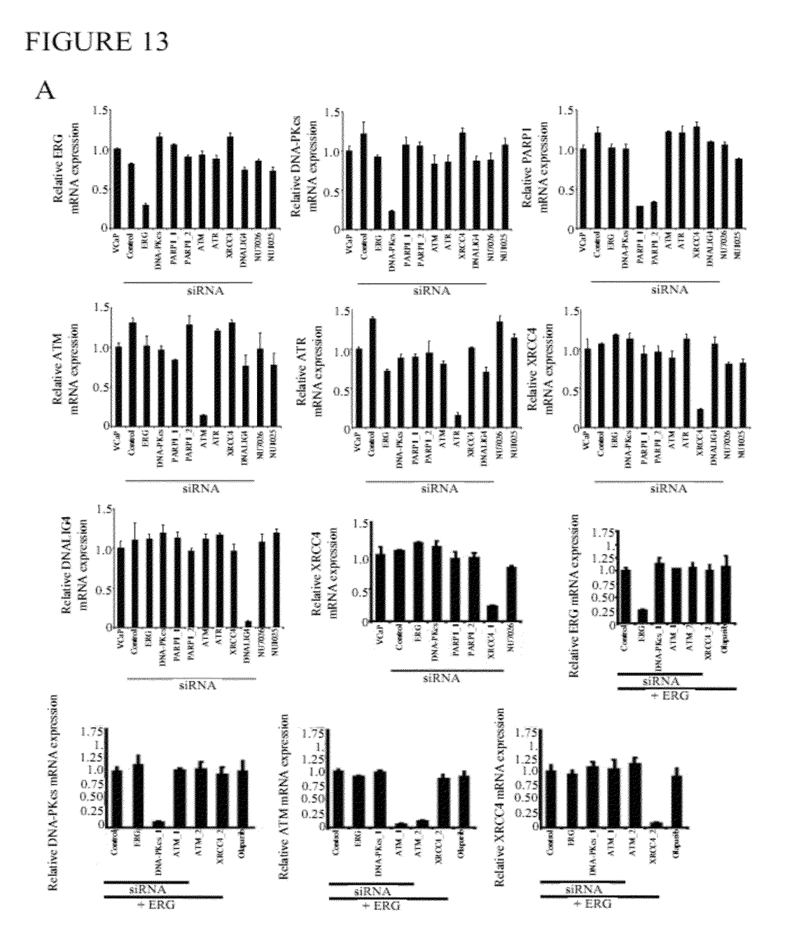

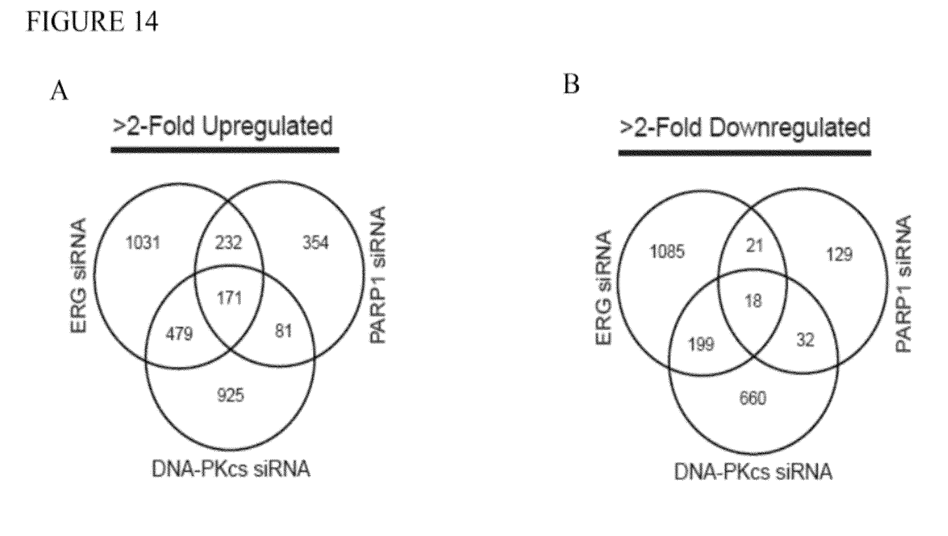

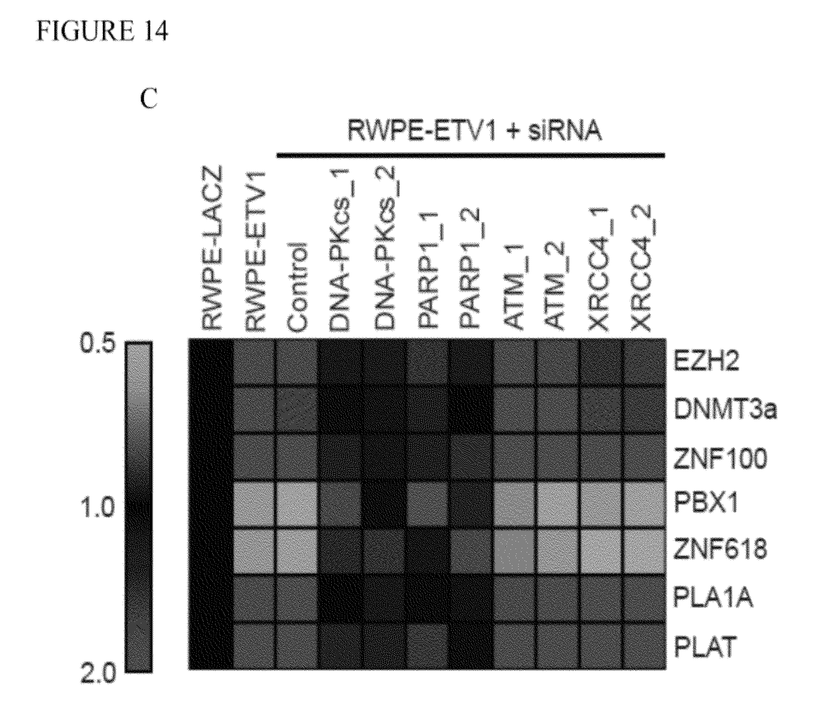

Given the importance of PARP1 and DNA-PKcs for ERG-mediated transcription of PLA1A in RWPE cells, the global effects of inhibiting PARP1 and DNA-PKcs on the ERG-transcriptome were investigated using Agilent Whole Genome Oligo Expression Arrays to profile RNA from VCaP cells treated with either DNA-PKcs or PARP1 siRNA (knockdown confirmed in FIG. 13). In replicate experiments, the analysis revealed 50 and 252 unique features that were greater than 2-fold down- and up-regulated, respectively, in both the PARP1 and DNA-PKcs siRNA-treated samples (Table 2 and 3, respectively). Venn diagram analysis was used to show the overlap of differential gene sets from either the ERG, PARP1 or DNA-PKcs siRNA treated VCaP cells (FIG. 14a, b) with statistical significance demonstrated using a hypergeometric test as indicated. To understand how this gene signature is related to existing ETS gene signatures, associations between more than 20,000 biologically related gene sets by disproportionate overlap were investigated using the Oncomine Concepts Map (OCM; Rhodes, et al. Neoplasia (New York, N.Y. 9, 443-454 (2007); Tomlins et al. Nature genetics 39, 41-51 (2007)). In other words, the enrichment analysis enables the identification of closely related gene sets from previously published gene expression experiments. The expression signature was uploaded into OCM to identify human tissue gene signatures that are enriched for genes regulated by DNA-PKcs and PARP1 in VCaP cells. By seeding the OCM analysis with the set of genes upregulated in both PARP1 and DNA-PKcs knockdown experiments (genes repressed by PARP1 and DNA-PKcs), the most significantly enriched concepts from the Oncomine database were identified. The most highly enriched signatures were those that are repressed in ETS positive as compared to ETS negative prostate cancer as previously reported by Xiaojun et al. (Tomlins et al. Nature genetics 39, 41-51 (2007)) [OR=3.08, P=1.40×10⁻¹⁵, Oncomine database], Yang et. al. (Tomlins et al. Nature genetics 39, 41-51 (2007)) [OR=2.91, P=3.30×100, Oncomine database] and Lapointe et. al. (Lapointe et al. Proceedings of the National Academy of Sciences of the United States of America 101, 811-816 (2004)) [OR=3.33, P=2.30×10⁻⁶] (FIG. 2c). This indicated that the set of genes that are repressed by DNA-PKcs and PARP1 in the VCaP cell line are also repressed by ERG in human prostate cancer tissues. The PARP1-DNA-PKcs repressed gene signature also showed significant overlap with the set of genes repressed in metastatic as compared to localized prostate cancer indicating that repression of these genes is important for prostate cancer progression; Vanaja et. al. (Cancer research 63, 3877-3882 (2003)) [OR=2.99, P=1.5×10⁻¹⁰], LaTulippe et. al. (Cancer research 62, 4499-4506 (2002)) [OR=3.31, P=1.50×10⁻⁶] (FIG. 2c). Treatment of VCaP cells with siRNA confirmed gene expression changes as predicted by the gene expression arrays (FIG. 2D), as did treatment with either the small molecule DNA-PKcs kinase inhibitor, NU7026, or the small molecule PARP1 inhibitor, Olaparib (FIG. 2E). Analysis of siRNA-treated RWPE-ETV1 cells (FIG. 12) confirmed that DNA-PKcs and PARP1 regulated ETV1 transcriptional activity as well (FIG. 2F). Taken together, the analysis demonstrated that both PARP1 and DNA-PKcs function as co-activators and co-repressors of ERG-driven transcription for the gene sets listed in Tables 2 and 3, respectively.

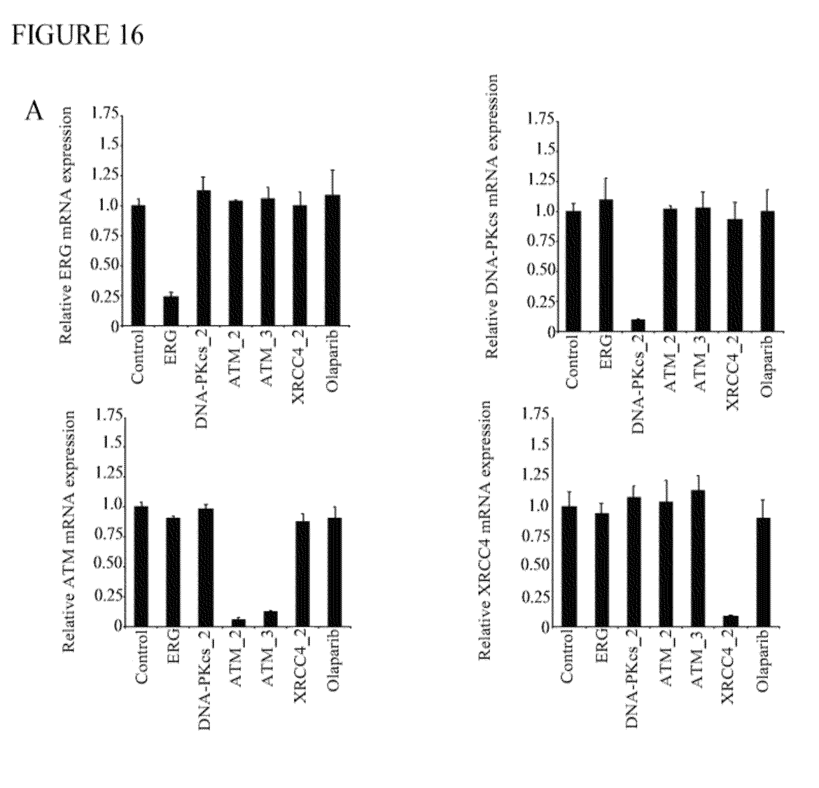

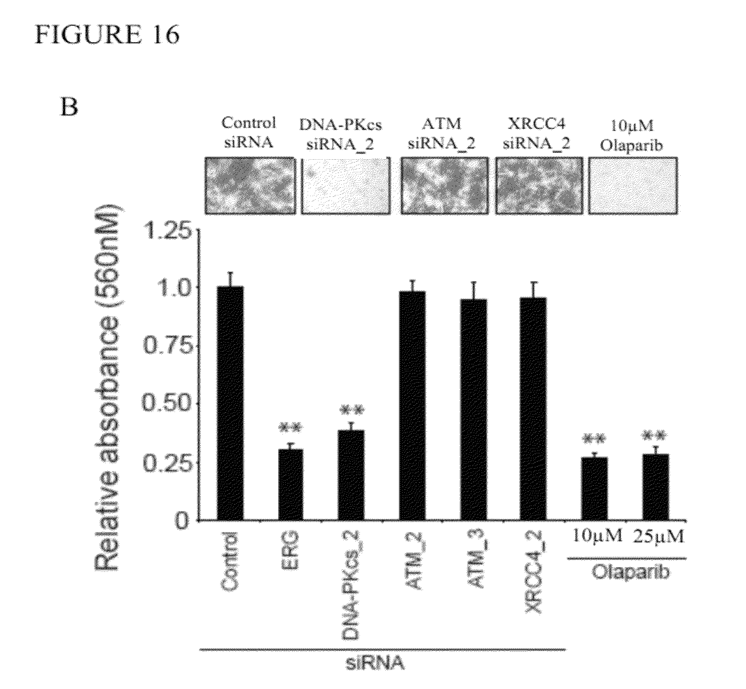

To then confirm the dependence of ERG-mediated transcription on PARP1 and DNA-PKcs expression and activity, a set of genes that were altered in the gene expression arrays (both activated and repressed) were selected. As shown in FIG. 2d, treatment of VCaP cells with either ERG, PARP1 or DNA-PKcs siRNA led to a significant change in gene expression of the ERG-regulated genes tested, as predicted by the gene expression arrays. Accordingly, treatment of VCaP cells with either the small molecule DNA-PKcs kinase inhibitor NU7026 or the small molecule PARP1 inhibitor Olaparib also altered expression of the ERG target genes (FIG. 2e). Although a change in the expression of the previously reported ERG target genes PLA1A and PLAT was observed with siRNA and drug treatments targeting either PARP1 or DNA-PKcs, the change was not as significant in VCaP cells as what was observed in the RWPE model. However, this is consistent with the fold changes observed in the gene expression array data. In order to confirm the role of PARP1 and DNA-PKcs in ETS-mediated transcription, qPCR analysis of these genes was performed in RWPE-ETV1 cells treated with siRNA and it was found that siRNA of either ETS-interacting kinase, but not the controls, blocked ETV1-mediated transcriptional activation of these targets (FIG. 14c). Knockdown was confirmed (FIG. 16a). Taken together the data indicates that the ETS:PARP1:DNA-PKcs complex plays a role in modulating ETS transcriptional activity of several genes including the invasion-associated gene EZH2.

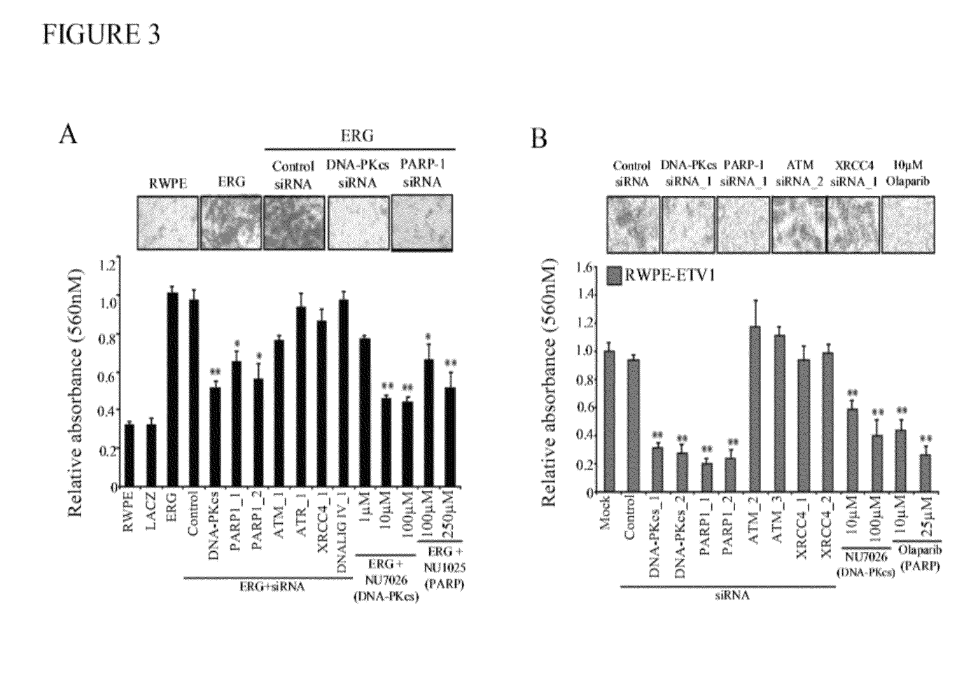

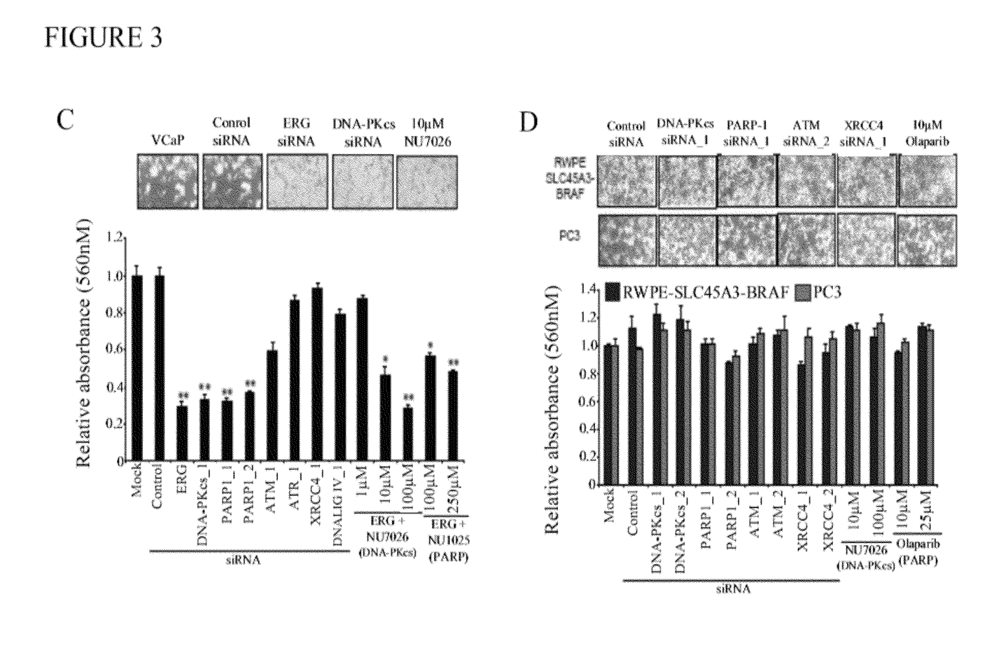

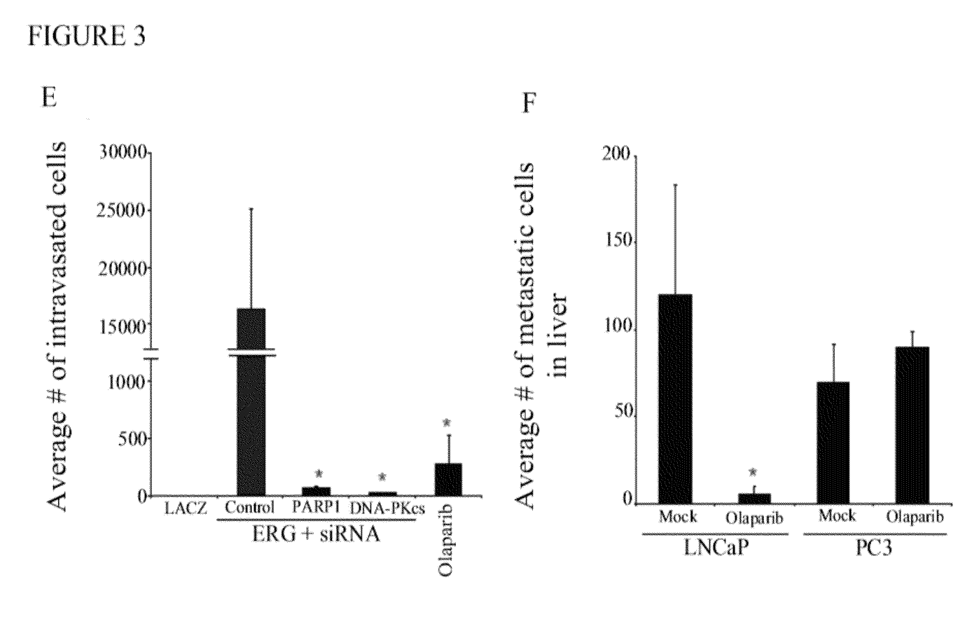

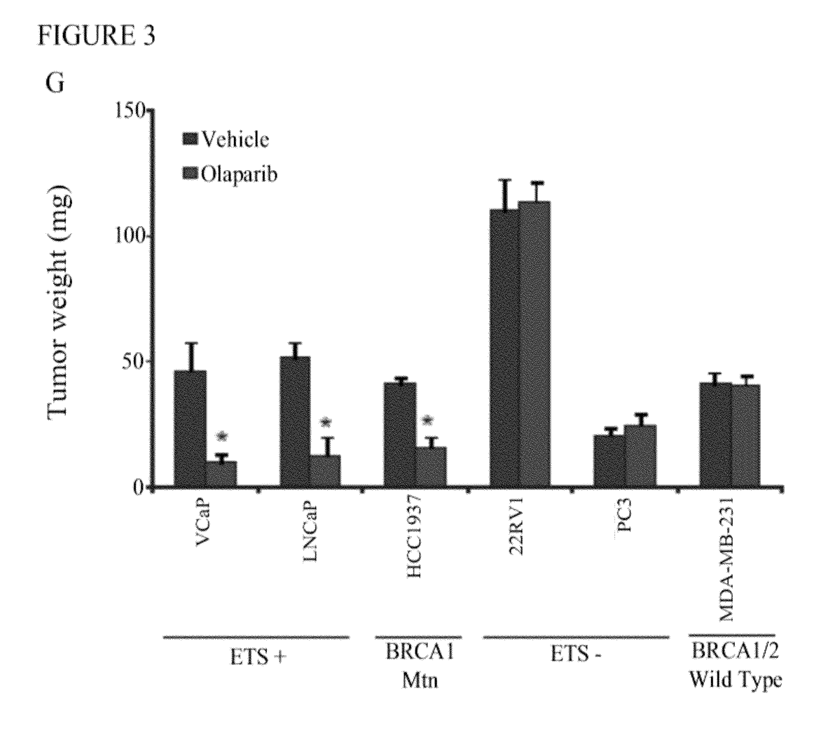

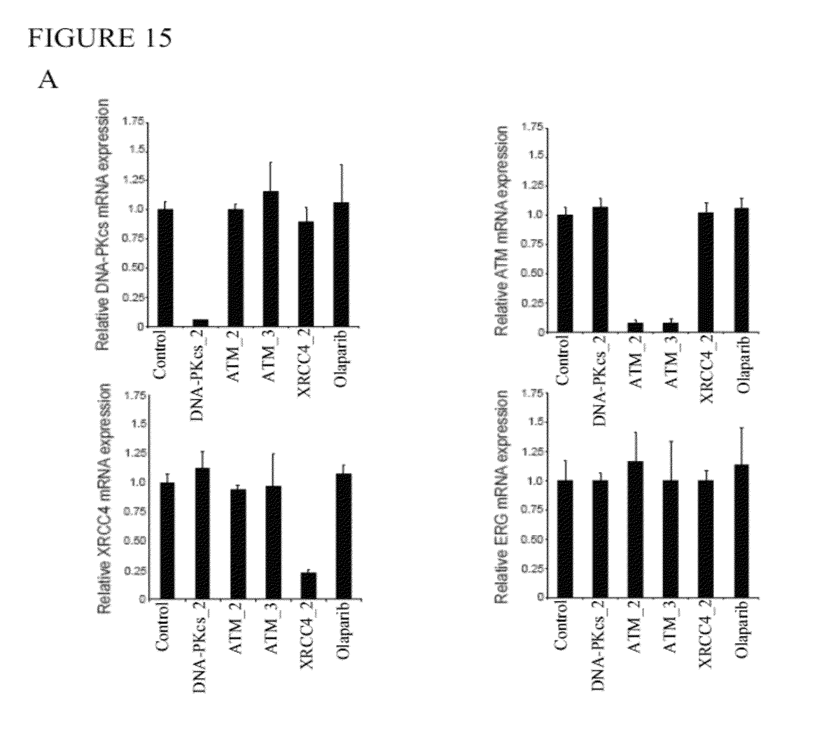

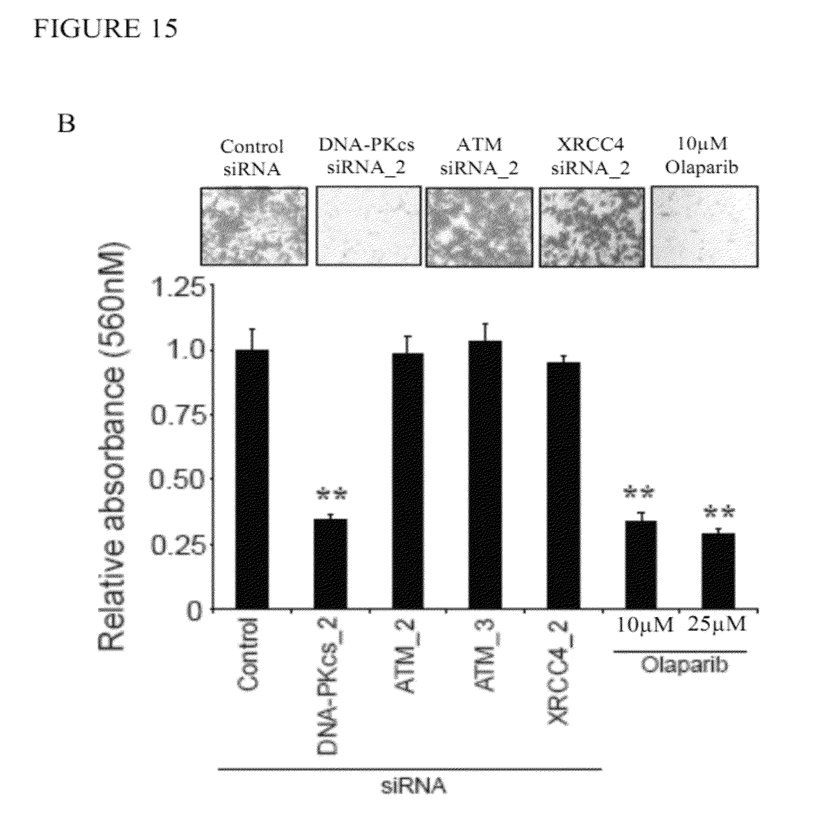

Previously, it was shown that ectopic expression of ERG in benign prostate epithelial cells leads to expression of an invasion-associated transcriptional program and increased invasion in matrigel coated transwell plates (Tomlins et al. Neoplasia (New York, N.Y. 10, 177-188 (2008)). Accordingly, because inhibition of PARP1 and DNA-PKcs altered ERG-transcriptional activity of several invasion associated genes like EZH2, the role of PARP1 and DNA-PKcs in ERG-induced cell invasion was assayed. Both DNA-PKcs siRNA and NU7026 attenuated invasion in both RWPE cells transduced with ERG adenovirus (FIG. 3a and FIG. 15) and VCaP cells (FIG. 3c and FIG. 16) [P<0.01 for DNA-PKcs siRNA or NU7026>10 μM]. Likewise, treatment with either PARP1 siRNA, the first generation PARP1 inhibitor, 8-hydroxy-2-methylquinazolinone (NU1025) (Bowman, K. J., Newell, D. R., Calvert, A. H. & Curtin, N. J. Differential effects of the poly (ADP-ribose) polymerase (PARP) inhibitor NU1025 on topoisomerase I and II inhibitor cytotoxicity in L1210 cells in vitro. British journal of cancer 84, 106-112 (2001)) or the second generation PARP1 inhibitor Olaparib, led to a significant reduction in ERG-driven and VCaP cell invasion (FIG. 3a, c and FIG. 15, 16) [P<0.05 for all PARP1 siRNA, NU1025 or Olaparib treatments]. This indicated that DNA-PKcs and PARP1 expression and enzymatic activity are required for ERG-induced invasion. As with the analysis of ERG-mediated transcription, knockdown of either ATM, ATR, XRCC4 or DNA Ligase IV did not have an effect on ERG-mediated or VCaP cell invasion (FIGS. 3a, c, 15 and 16). All siRNA knockdown efficiencies were confirmed (FIG. 12, 13, 15, 16). This data was consistent with the observation that ATM and ATR were not required for ERG-mediated transcription and that transcription did not depend on the execution of NHEJ.

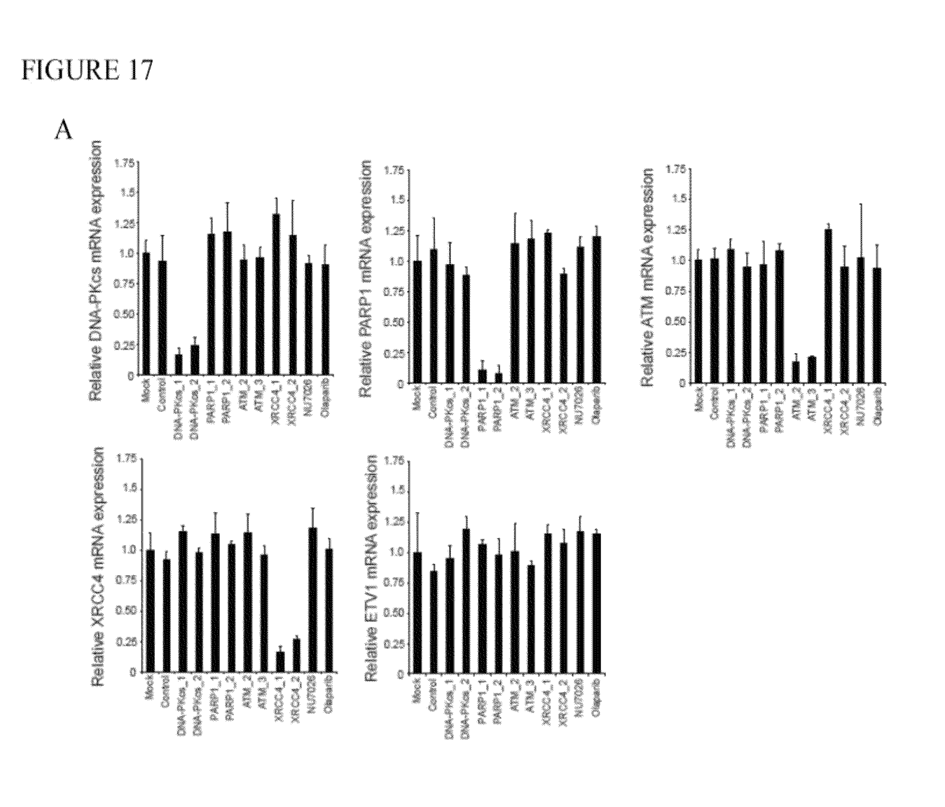

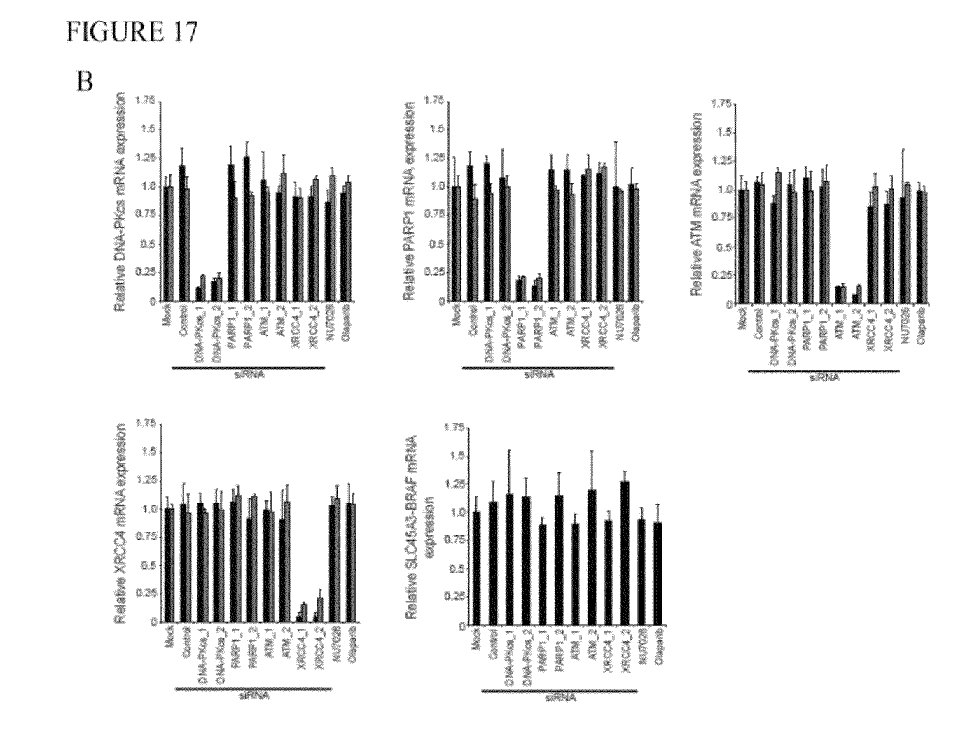

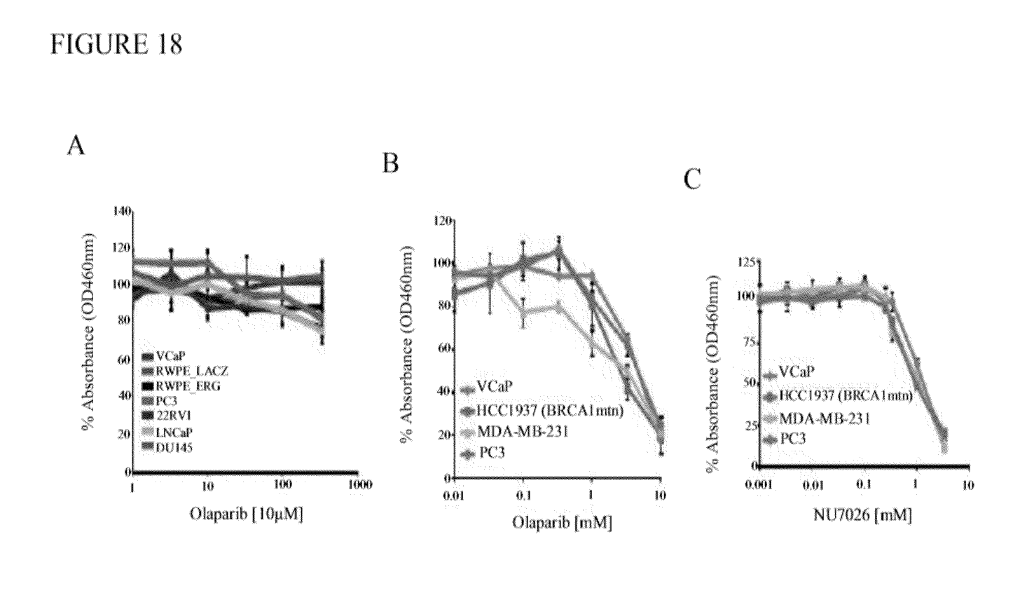

To extend the observation that PARP1 and DNA-PKcs are required for ERG-mediated invasion to other ETS models, RWPE cells stably overexpressing ETV1 (Tomlins, S. A., et al. Nature 448, 595-599 (2007)) were treated with PARP1 or DNA-PKcs siRNA or small molecule inhibitors. A significant reduction in ETV1-mediated invasion (P<0.01 for all PARP1 or DNA-PKcs treatments) was observed (FIG. 3b), while the negative control siRNAs did not alter ETV1-mediated invasion. Knockdown efficiency was confirmed (FIG. 17a). Invasion of two negative control models, the ETS rearrangement negative cell line PC3 and RWPE cells overexpressing a prostate cancer oncogene that drives invasion called SLC45A3-BRAF (Palanisamy et al. Nature medicine 16, 793-798), was not affected by either PARP1 or DNA-PKcs siRNA or respective small molecule inhibitors (FIGS. 3d and 17b). Olaparib treatment did not have an effect on the in vitro cell proliferation rate of any of the cell lines tested as assessed after 72 hours by WST chemosensitivity assay indicating that the reduction in cell invasion is not due to changes in cell proliferation (FIG. 18). Accordingly, the PARP1 and DNA-PKcs siRNA treatments as well as NU7026 did not alter total cell number after 72 hours (data not shown). Taken together this data indicates that PARP1 and DNA-PKcs are specifically required for ETS-mediated invasion.

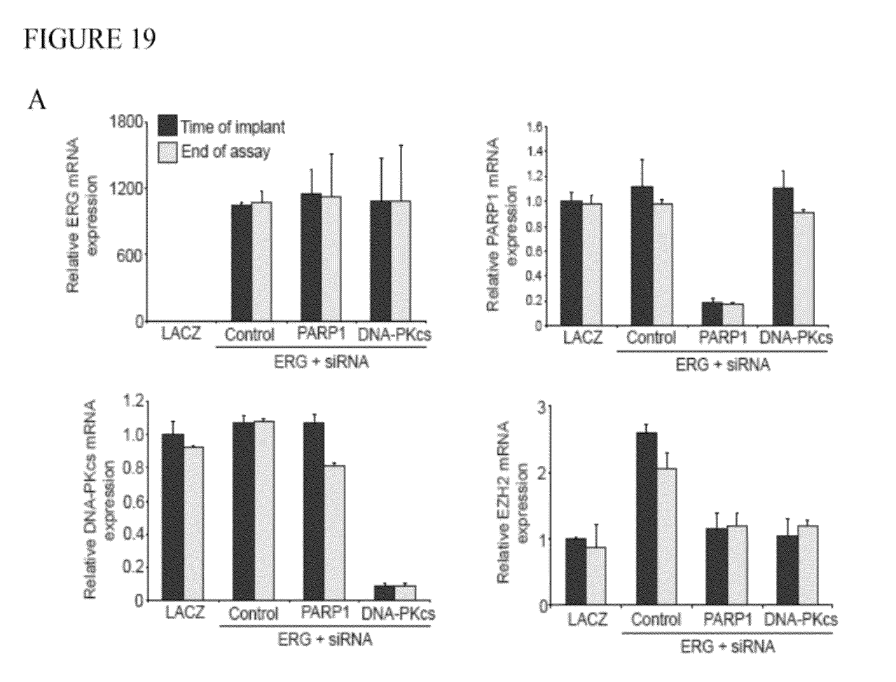

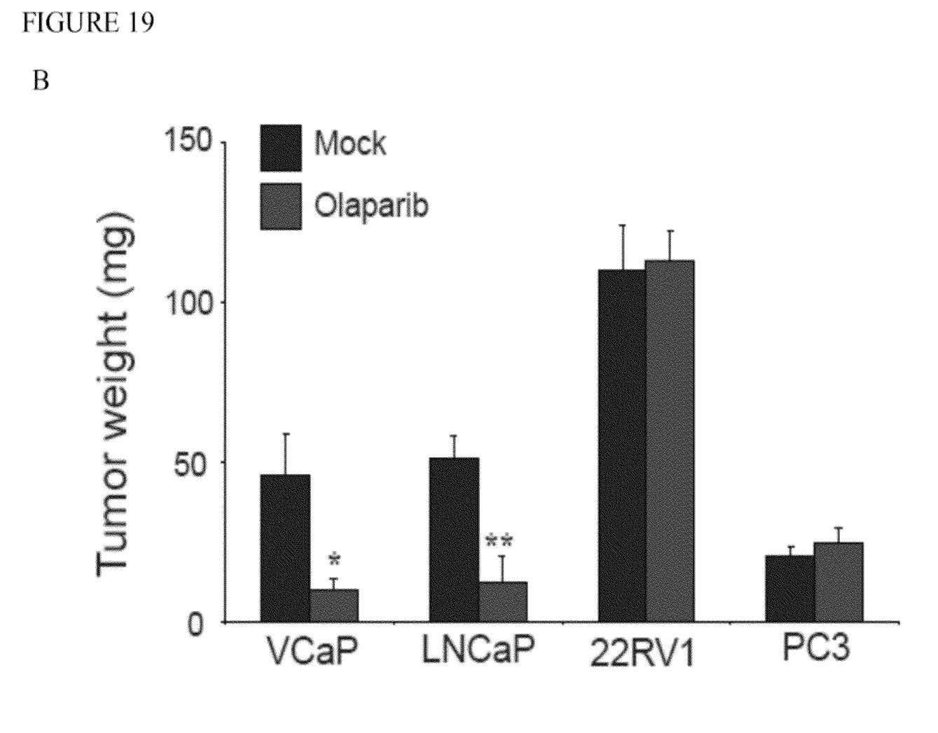

As such, the role of PARP1 in ERG-mediated metastasis was analyzed in ERG-mediated intravasation. To do this, cells were implanted onto the upper chorioallantoic membrane (CAM) of a fertilized chicken embryo and the relative number of cells that intravasate into the vasculature of the lower CAM three days after implantation was analyzed (Kim et al., Cell 94, 353-362 (1998)). In line with the invasion assays, Olaparib treatment was sufficient to block ERG-mediated intravasation (P<0.1). Similarly, because the intravasation assay is performed over a short time-course, the effects of treating RWPE-ERG cells with the most efficient PARP1 or DNA-PKcs siRNA were assessed and it was found that both siRNAs blocked ERG-mediated intravasation (P<0.1). Knockdown was monitored over the course of the experiment (FIG. 19a). EZH2 mRNA expression was monitored throughout the intravasation experiment. EZH2 expression was downregulated following either PARP1 or DNA-PKcs inhibition (FIG. 19a). This indicated that mechanistically PARP inhibition may disrupt ERG-mediated intravasation by inhibiting ERG-mediated transcriptional activation of EZH2. Therapeutic disruption of either ERG-interacting enzymes (PARP1 or DNA-PKcs) is also contemplated to prevent the metastatic spread of prostate cancers harboring ETS gene fusions.

To test the hypothesis that PARP1 activity is required to drive the metastatic spread of ETS positive cells, a panel of ETS positive and ETS negative cell lines treated with Olaparib were analyzed. In this assay, cells are implanted above the upper CAM of a fertilized chicken embryo, eggs are treated every other day with Olaparib and metastasis is assessed by quantifying the number of cells that metastasize to the liver over a prolonged incubation period (Asangani et al. Oncogene 27, 2128-2136 (2008); Luo, J. L., et al. Nature 446, 690-694 (2007)). As shown in FIG. 3f, Olaparib treatment blocked the formation of liver metastases in the ETS positive cell line LNCaP (P=0.1), but not the ETS negative cell line PC3. No detectable metastatic cells were present in the livers of animals xenografted with either 22RV1 or VCaP cells indicating that these cell lines are either not metastatic or that they do not metastasize to the liver. Over the extended treatment period the ETS-positive tumors were significantly smaller than the ETS-negative tumors (FIG. 19b) with P<0.05 for VCaP and P<0.01 for LNCaP. This indicates that PARP1 could play a role in the long term maintenance of ETS-positive cancer cell survival.

Because the long-term survival of ETS-overexpressing tumors can be diminished by treatment with Olaparib, the magnitude of effect was compared to that of a clinically validated model. Therefore, HCC1937 (BRCA1 mutant) and MDA-MB-231 (BRCA1/2 WT) cell lines were zenographted, and following Olaparib treatment a significant effect was observed on the BRCA1 mutant HCC1937 tumors, whereas no measurable effect was observed in MDA-MB-231 tumors. The magnitude of effect observed in the HCC1937 cells was equivalent to the magnitude of effect observed in the two ETS-positive cell line xenografts (FIG. 3g).

ETS Positive Xenografts are Susceptible to PARP1 Inhibition

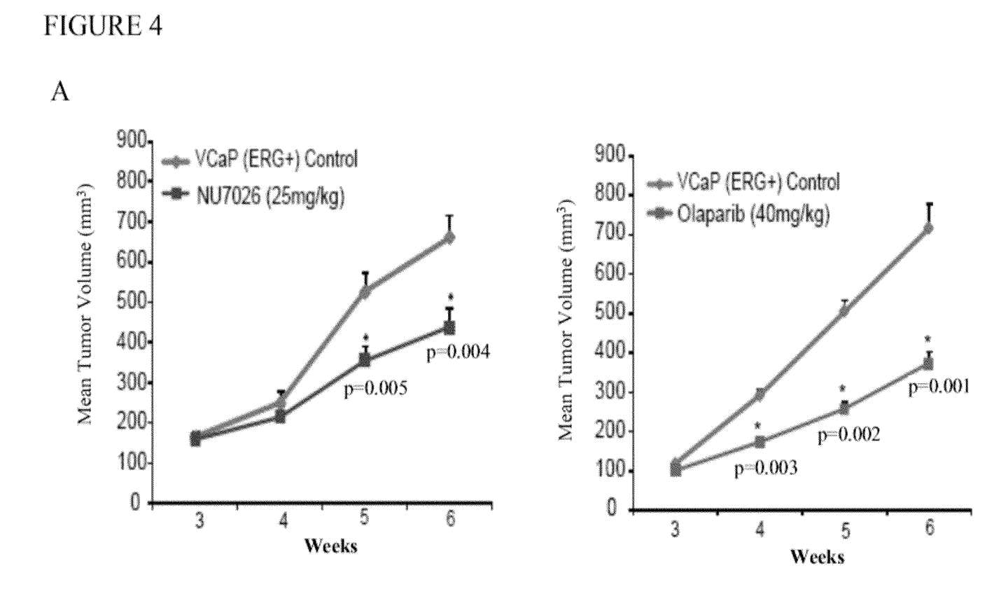

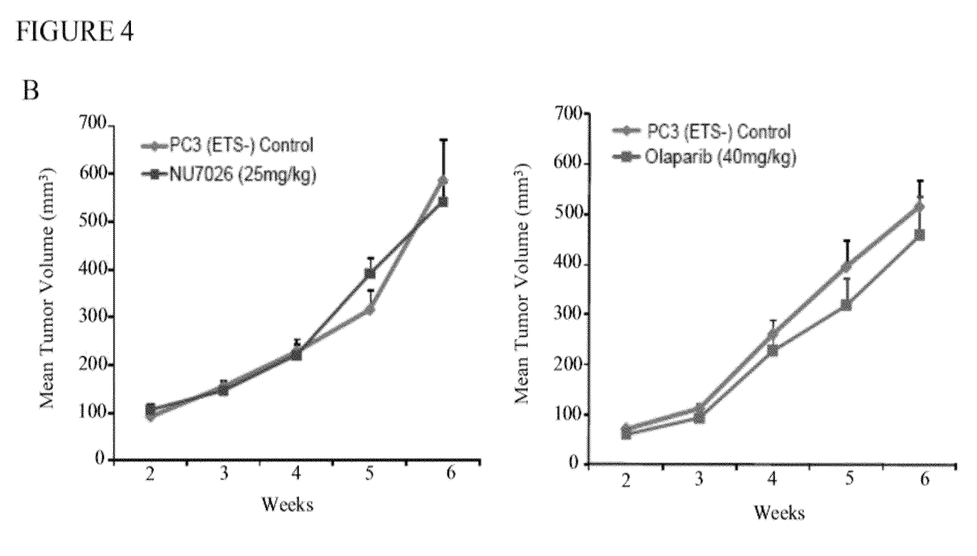

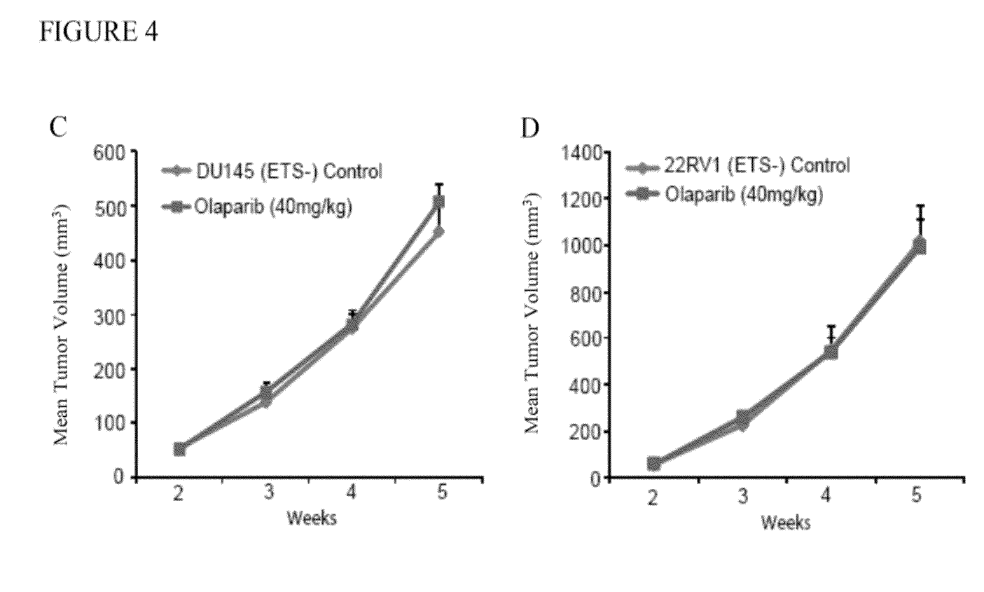

Based upon in vivo data from the chicken CAM assay, it was contemplated that inhibition of either PARP1 or DNA-PKcs would inhibit ETS-positive prostate cancer growth in a mouse xenograft model. Several PARP inhibitors have recently entered Phase I and Phase II clinical trials. One of these, Olaparib, was shown to be well tolerated in cancer patients with a minimal side effect profile (Fong et al. The New England journal of medicine 361, 123-134 (2009)). VCaP cells were injected into the flank of the mouse and Olaparib (40 mg/kg/day, IP) or NU7026 (25 mg/kg/day, IP) were administered. A significant reduction of tumor growth relative to the vehicle control was observed in all treatment groups (FIG. 4a) (P=0.001 for Olaparib treated and p=0.004 for NU7026 treated). Olaparib or NU7026 did not block the growth of the ETS negative PC3 xenografts (FIG. 4b) indicating preferential sensitivity of ETS positive tumors. This was further substantiated in 2 additional ETS negative DU145 and 22RV1 xenografts which were similarly not responsive to PARP inhibition (FIG. 4c, d).

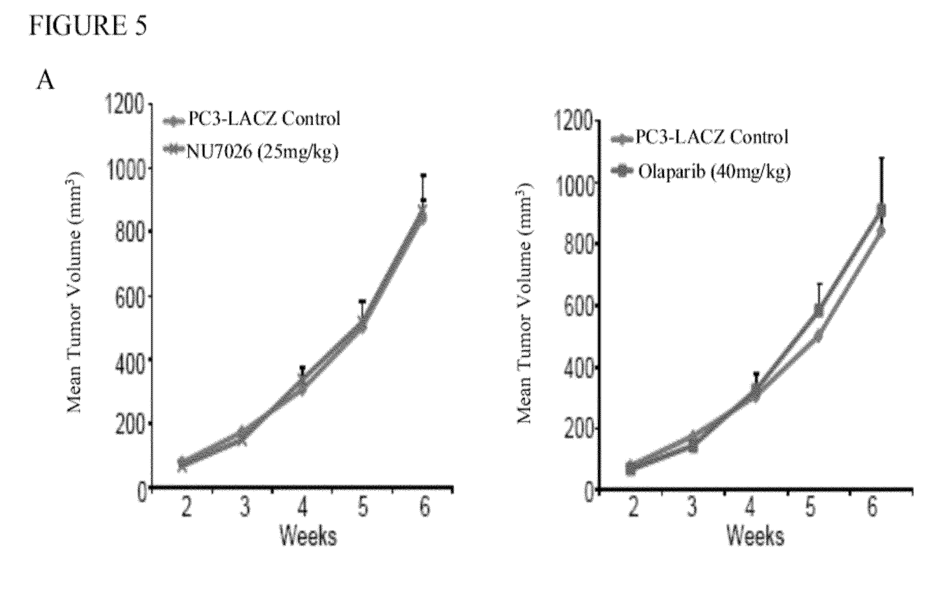

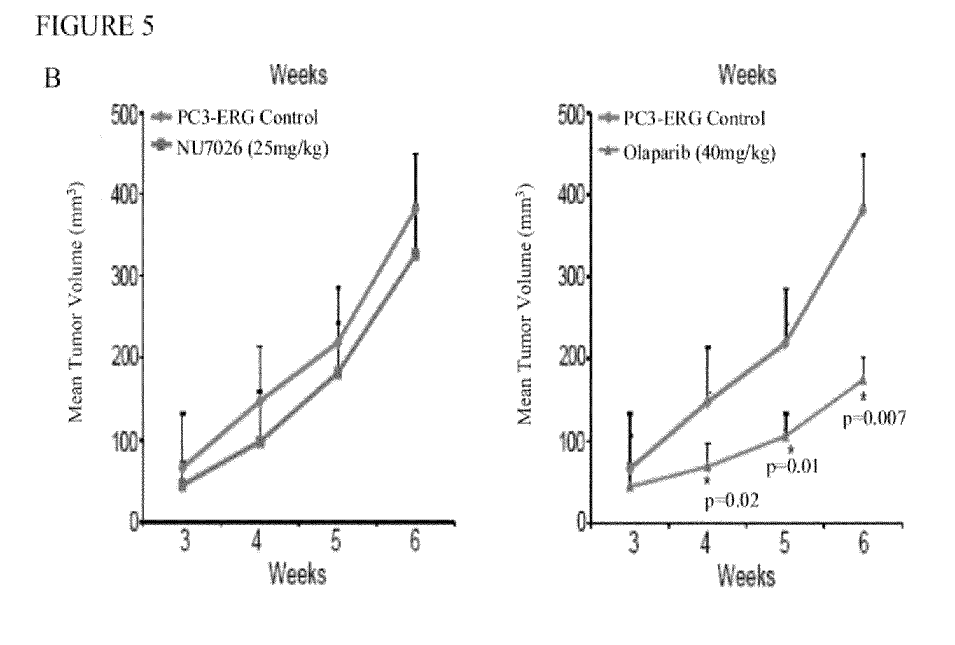

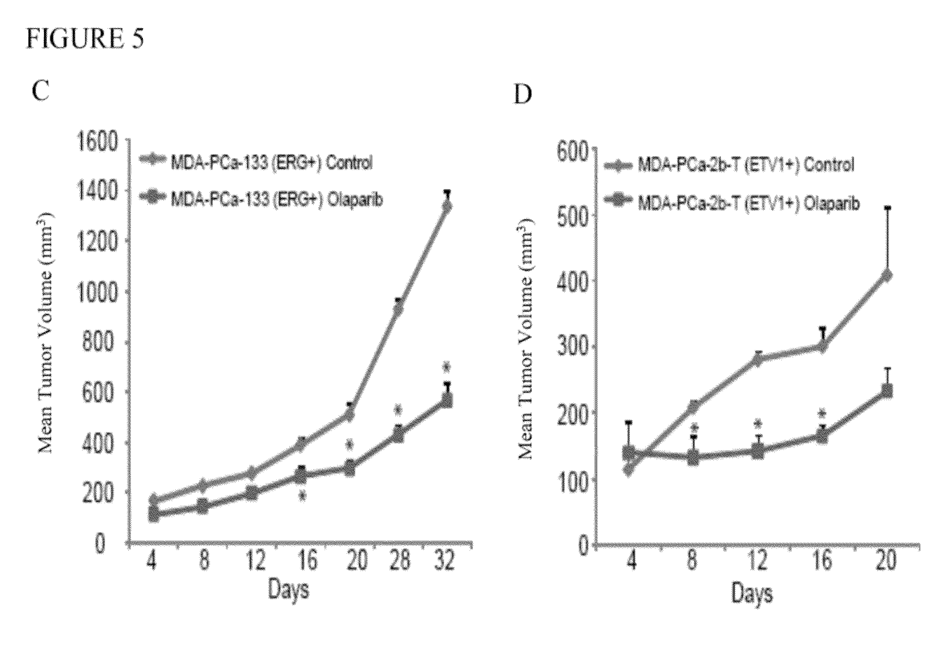

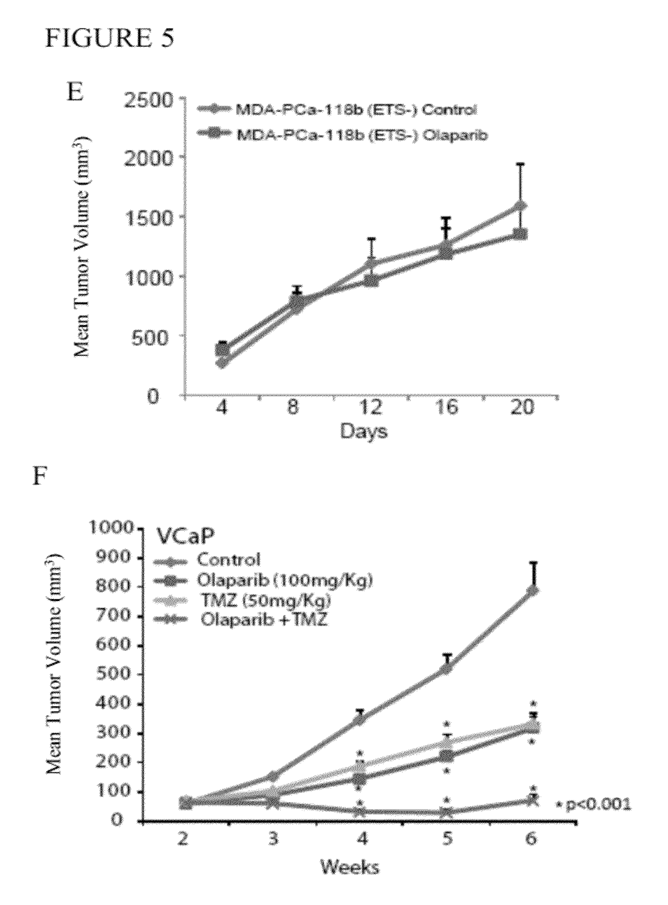

The experiment was then extended to analyze the effects of Olaparib on a panel of ETS-positive and ETS-negative prostate cancer cell lines, including an isogenic model. Because this experiment intended to test the specificity of Olaparib-induced growth inhibition for ERG-overexpressing prostate xenografts, a dose similar to that used in previously published xenograft experiments was utilized (Rottenberg et al., Proc. Natl. Acad. Sci. USA 105, 17079-17084 2008). This dose of Olaparib had a significant effect on VCaP cells (p=0.001) but did not inhibit the growth of two additional ETS-negative cell line xenografts (22RV1 or DU145) (FIGS. 5A-5C). This experiment also demonstrated that the VCaP tumor growth response was dose dependent.

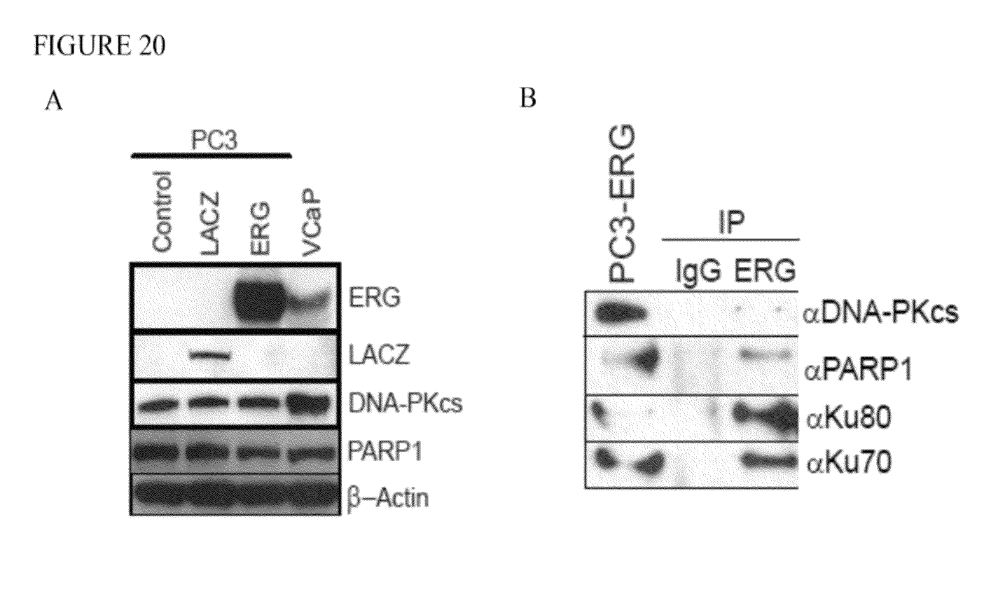

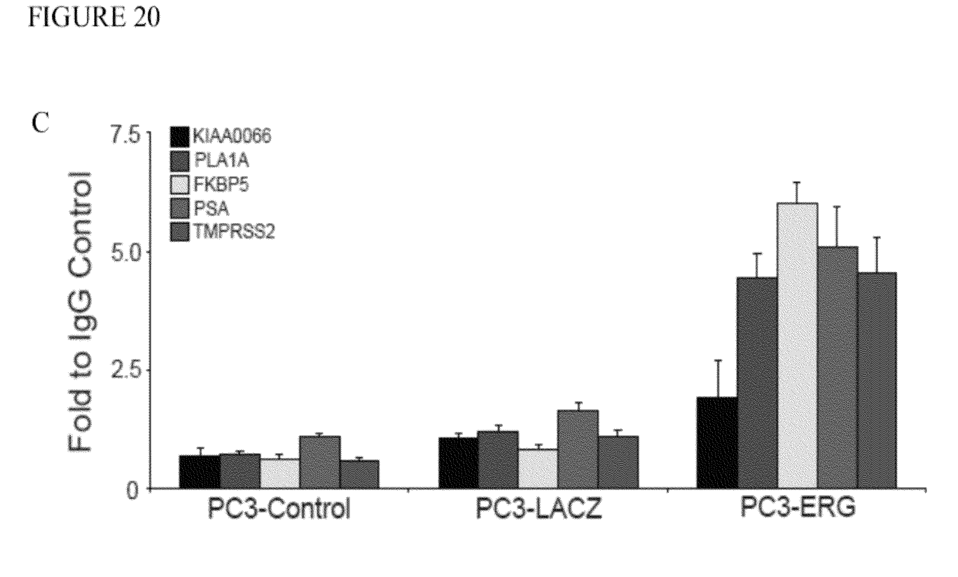

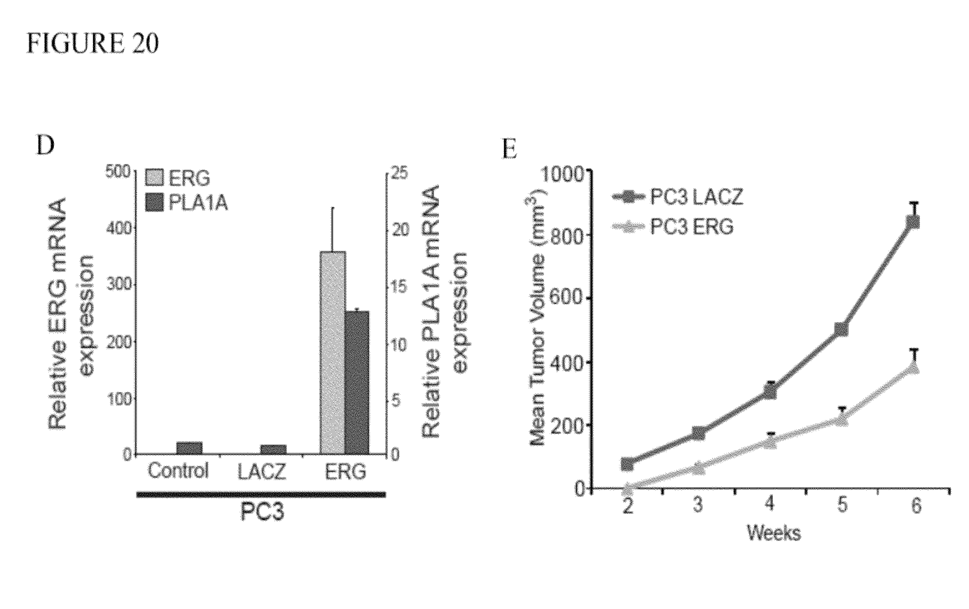

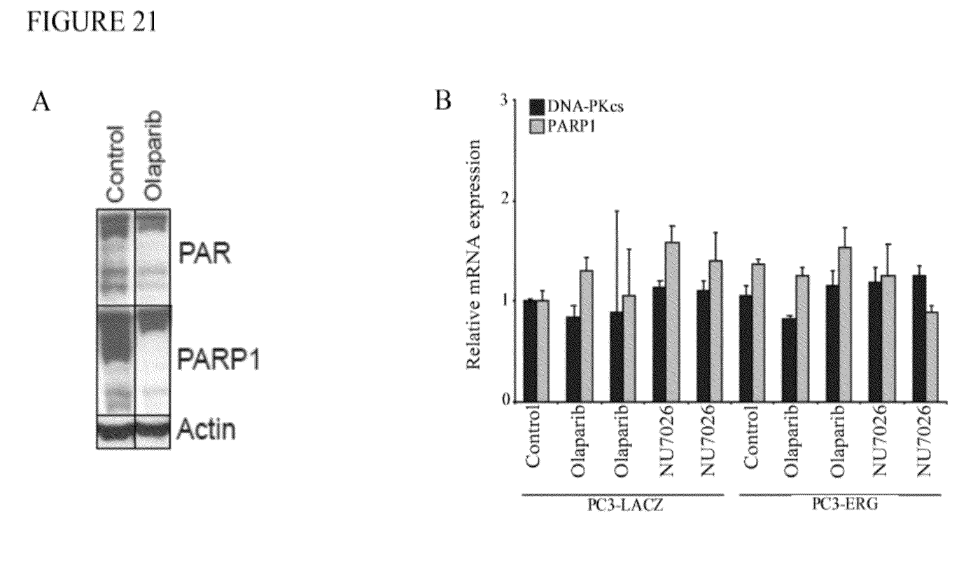

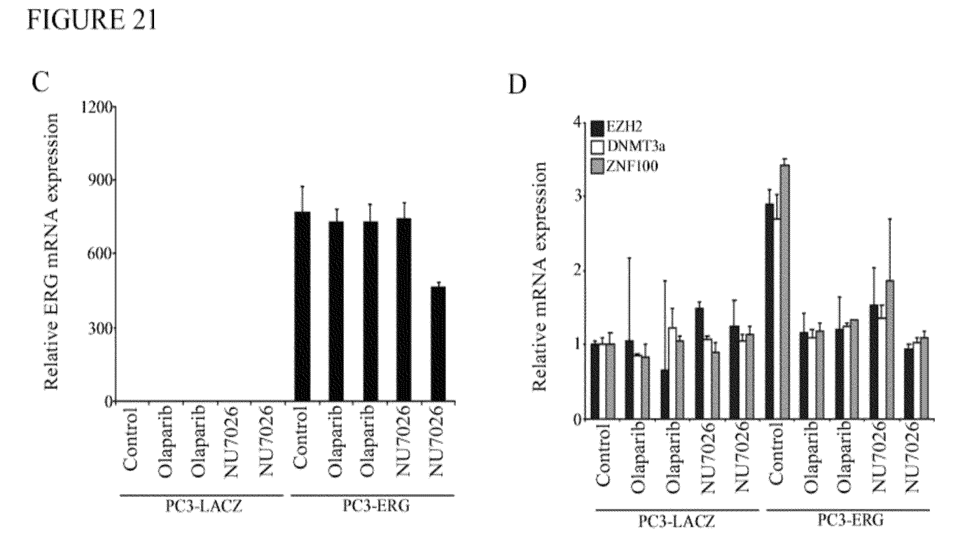

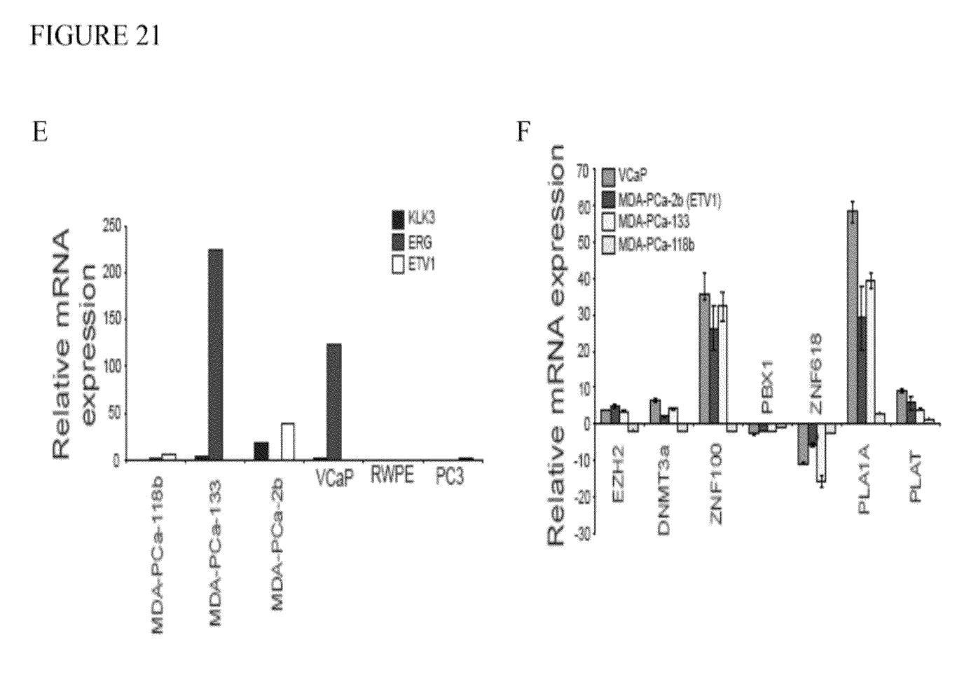

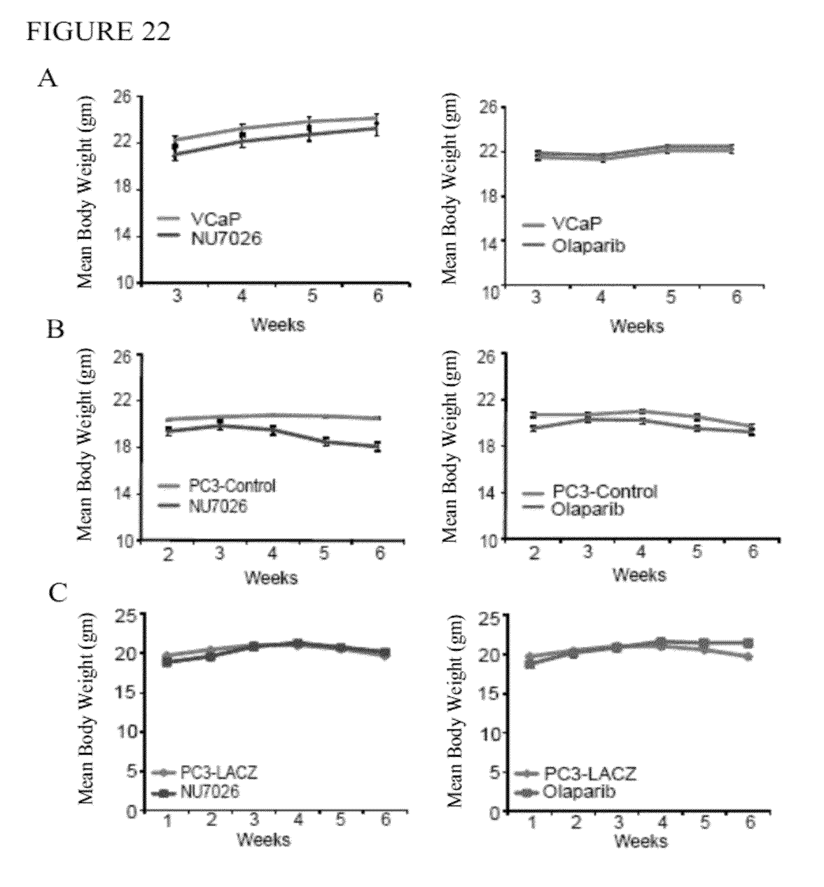

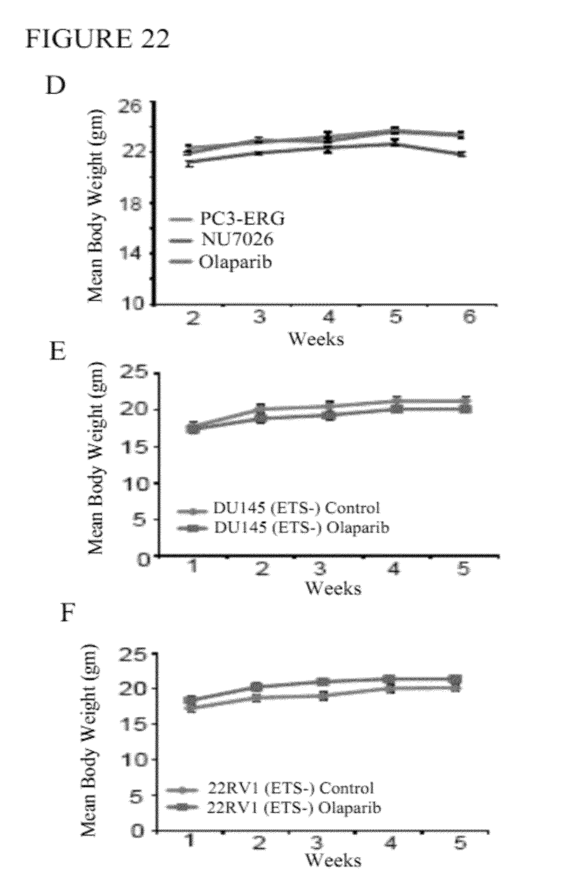

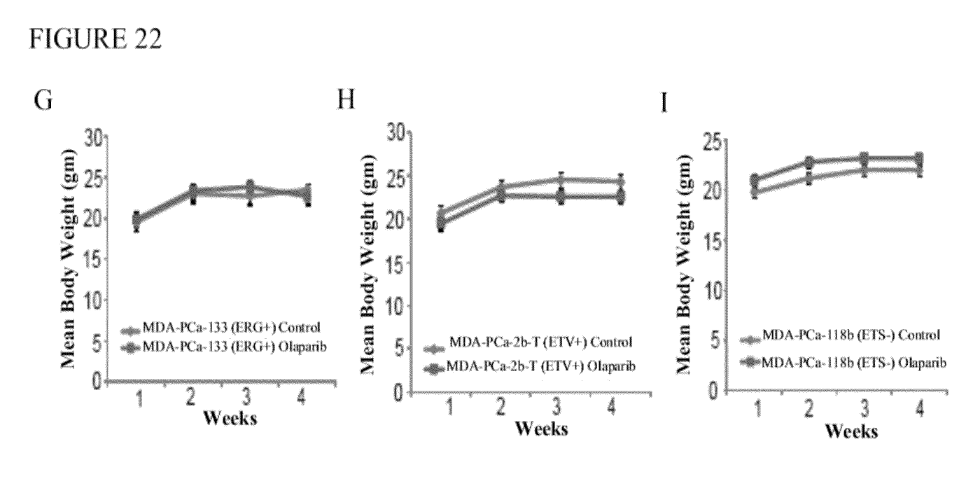

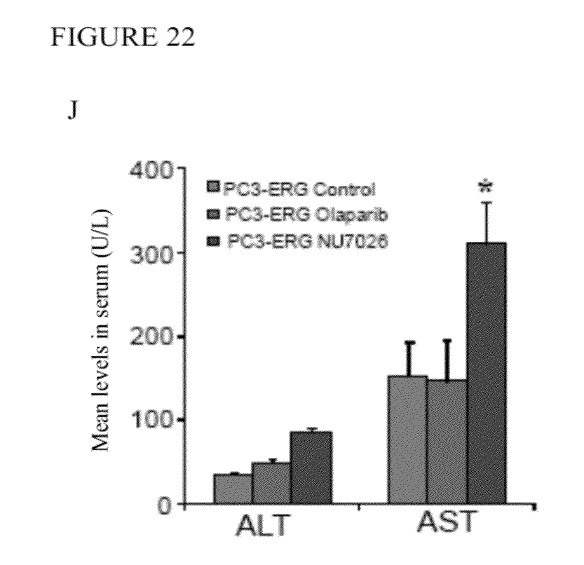

To extend these findings into an isogenic model, an ETS-positive system was generated by overexpressing the coding sequence of the primary TMPRSS2:ERG gene fusion product reported in prostate cancer (Brenner et al. Head & neck (2009)) in the PC3 prostate cancer cell line (PC3-ERG). Luciferase alone was used as a lentiviral vector control (PC3-Control) as well as LACZ (PC3-LACZ). Western blotting was used to confirm ERG protein overexpression and immunoprecipitation-Western blot to confirm that ERG interacted with both PARP1 and DNA-PKcs in this cell line model (FIGS. 20a and b). Likewise, ChIP assays demonstrated that ERG binds to known target genes in PC3 cells (FIG. 20c). Subsequent qPCR analysis revealed increased expression of both the ERG transcript and a previously reported ERG target gene, PLA1A (FIG. 20d). After confirming the functional activity of ERG in the PC3 cell line, the cell lines were injected subcutaneously into mice. ERG overexpression led to a slightly reduced growth rate of PC3 cells relative to LACZ overexpressing cells (FIG. 20e). Consistent with the model that ETS-positive, but not ETS-negative prostate tumors are susceptible to PARP or DNA-PKcs inhibition, Olaparib and NU7026 did not significantly inhibit xenograft growth of the ETS negative PC3-control or PC3-LACZ cell lines (FIGS. 4b and 5a, respectively). Overexpression of ERG was sufficient to significantly sensitize the PC3 cells to PARP inhibition (P=0.007) indicating that ERG overexpression provides selective Olaparib-mediated synthetic inhibition (FIG. 5b). Western blot and qPCR analysis of flash frozen, staged PC3-ERG tumors treated with or without drug for 4 hours confirmed inhibition of PARP1 activity by Olaparib and the loss of ERG-target gene expression after treatment with either inhibitor (FIG. 21a, b, c, d). In line with the clinical observation that Olaparib is well tolerated at doses capable of inhibiting PARP activity (Fong, supra) the Olaparib treatments in the xenograft model did not lead to a significant decrease in total body weight and did not lead to liver toxicity as assessed by serum levels of alanine aminotransferase (ALT) and aspartate aminotransferase (AST) (FIG. 22). NU7026 was unable to reduce the xenograft growth rate.

Given the specific inhibition of ERG positive cell line xenograft growth by Olaparib, the analysis was extended with the use of a model of primary human prostate tumors maintained in serial xenografts (Li et al. The Journal of clinical investigation 118, 2697-2710 (2008)) called “primagrafts”. Using this model, the effects of human tumors that have never been in cell culture were assessed. One ERG positive (MDA-PCa-133), one ETV1 one positive (Tomlins, et al. Nature 448, 595-599 (2007)) (MDA-PCa-2b-T) and one ETS negative (MDA-PCa-118b) primagraft model were assessed by assessing relative levels of ETS gene expression by qPCR (21e). Functional ETS gene status was assessed by testing the relative expression of several ETS target genes including EZH2, DNMT3a, ZNF100, PBX1 ZNF618 PLA1A and PLAT, between the models (FIG. 21f). As shown in FIGS. 5c and 5d, Olaparib inhibited the growth of both the ERG and ETV1 overexpressing primary human prostate cancer primagrafts [for MDA-PCa-133: P=0.05 (Day 8) and P<0.01 (day 16, 24 and 32) for MDA-PCa-2b-T: P<0.01 (day 8, 12 and 16)], but had no effect on the ETS-negative primagraft model (FIG. 5e). This again indicated that ETS-positive prostate cancers are specifically susceptible to PARP inhibition. In total, it was found that 4 ETS-positive (ERG or ETV1) xenografts (including primagrafts) were susceptible to PARP inhibition and 5 ETS-negative xenografts were completely resistant. In all cases, Olaparib did not have an observable effect on total body weight (FIG. 22a-i).

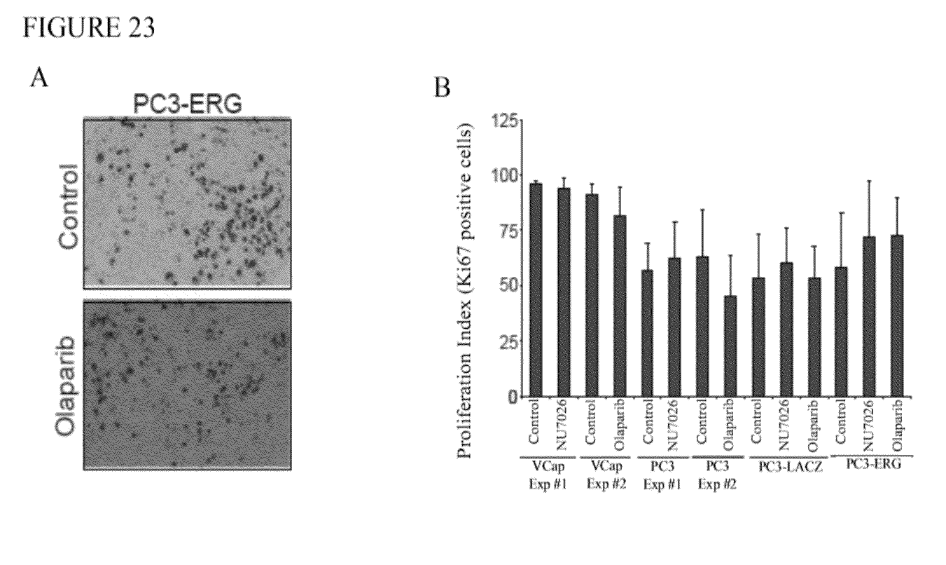

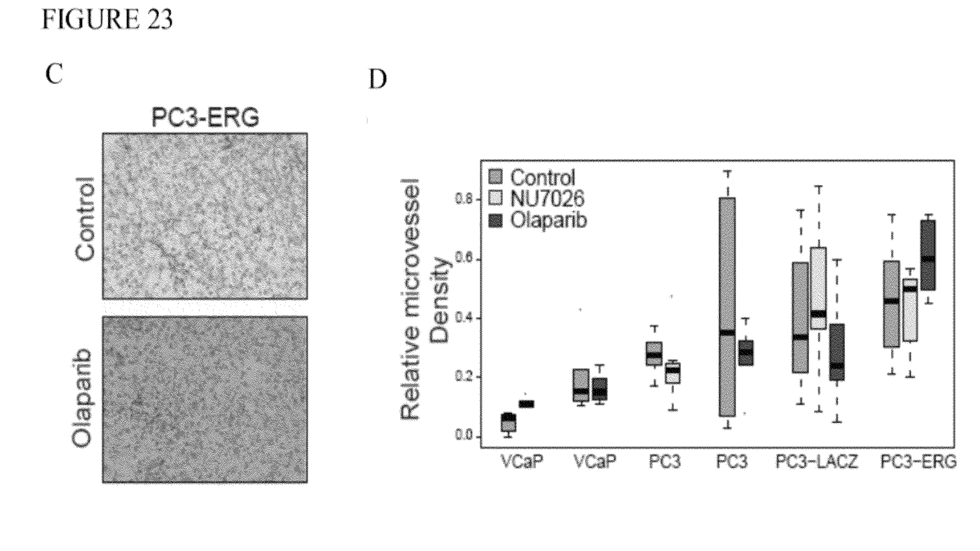

Changes in both relative cell proliferation of the tumors (Ki67) and microvessel density (CD31) were assessed using a tissue microarray made from formalin-fixed, paraffin-embedded tumors from all xenograft tumors analyzed in the VCaP and PC3 studies. No change was observed in either the average number of Ki67 positive cells or the microvessel density in the PC3, PC3-LACZ or PC3-ERG xenografts indicating that the addition of ERG to the already highly aggressive PC3 genetic background does not further enhance these phenotypes. Neither NU7026 nor Olaparib consistently led to a significant change in either the average number of Ki67 positive cells or the density of microvessels for the tumors tested (pairwise t-test) indicating that the mechanism (the present invention is not limited to a particular mechanism; nor is an understanding of the mechanism necessary to practice the present invention) by which Olaparib specifically inhibits the growth of ETS positive tumors is independent of both tumor cell proliferation and microvessel density (FIG. 23).

The treatment regimen was extended to identify combination therapies that enhance the magnitude of inhibition without causing significant toxicity. Recently, an alkylating agent called temozolomide (TMZ) has been shown to potentiate the effects of other PARP inhibitors in several cancer xenograft models (Donawho et al., Clin. Cancer Res. 13, 2728-2737 2007; Liu et al., Mol. Cancer Res. 6, 1621-1629 2008; Palma et al., Clin. Cancer Res. 15, 7277-7290 2009) as well as caused a complete or partial response in some patients enrolled in a phase II trial for metastatic breast cancer (S. J. Isakoff et al., 2010, J. Clin. Oncol., abstract). The combination treatment resulted in a significant growth reduction of VCaP tumors that was maintained over the entire 6 weeks (p<0.001 for all combination treatments) (FIG. 50. Even with the combination therapy, at this dose range, no overt toxicity such as excessive weight loss was observed (FIG. 22). This indicates that the addition of PARP inhibitor therapy to existing chemotherapeutic regimens will help enhance the overall effect for ETS-positive tumors.

ETS Transcription Factors Drive DNA Double Strand Break Formation

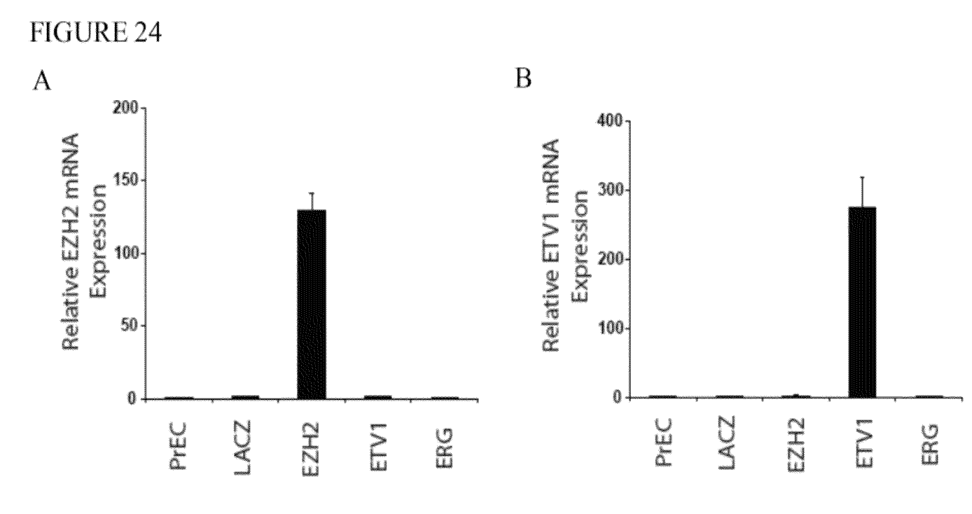

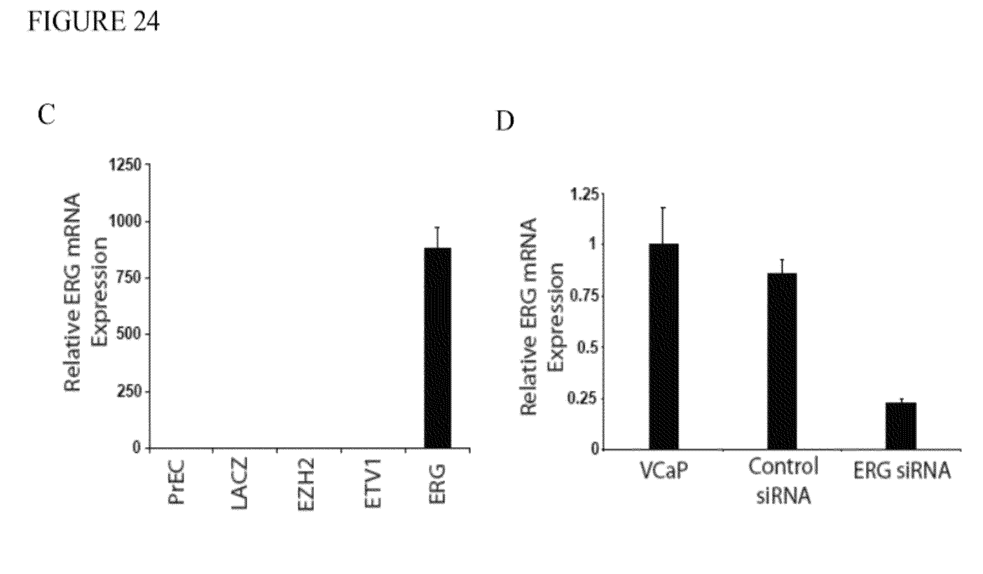

Total levels of DNA double strand breaks were assessed in vitro. The total levels of a histone marker of DNA double strand breaks called γ-H2A.X (Histone 2A.X phosphorylated on S139) was assessed in Olaparib-treated versus untreated VCaP cells. The untreated cells had a high level of γ-H2A.X foci (FIG. 6a), indicating that overexpression of ETS genes may induce DNA double strand breaks. To test this, primary prostate epithelial cells (PrEC) cells (FIG. 6a, b) were infected with the control genes, LACZ or EZH2 (an oncogene known to induce invasion in PrEC cells (Matsuoka et al. Science (New York, N.Y. 316, 1160-1166 (2007))), or the ETS gene fusion products, ERG or ETV1, using lentiviral expression vectors. In these benign prostate cells, ERG and ETV1 induced over 5 γ-H2A.X foci in greater than 75% of the analyzed cells while overexpression of either LACZ or EZH2 exhibited only 10% or less of cells with a similar level of foci formation, which was also observed in mock infected cells (FIG. 6a, b). Quantitative PCR was used to confirm changes in mRNA expression of EZH2, ETV1 and ERG (FIG. 24a-c). To then confirm that ERG induces γ-H2A.X in an endogenous setting, ERG was depleted from VCaP cells by RNA interference (FIG. 24d) significantly decreased γ-H2A.X foci, P=7.16×10⁻³ was observed (FIG. 6a, b).

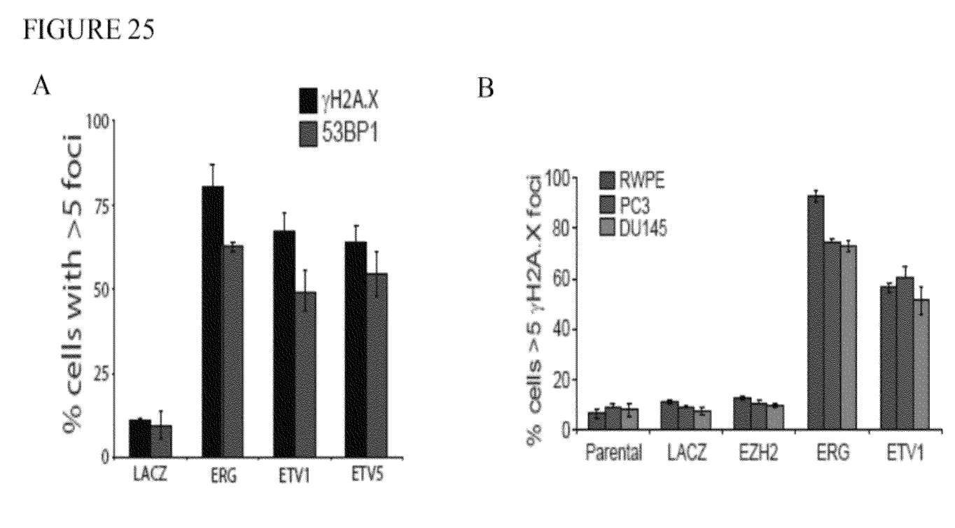

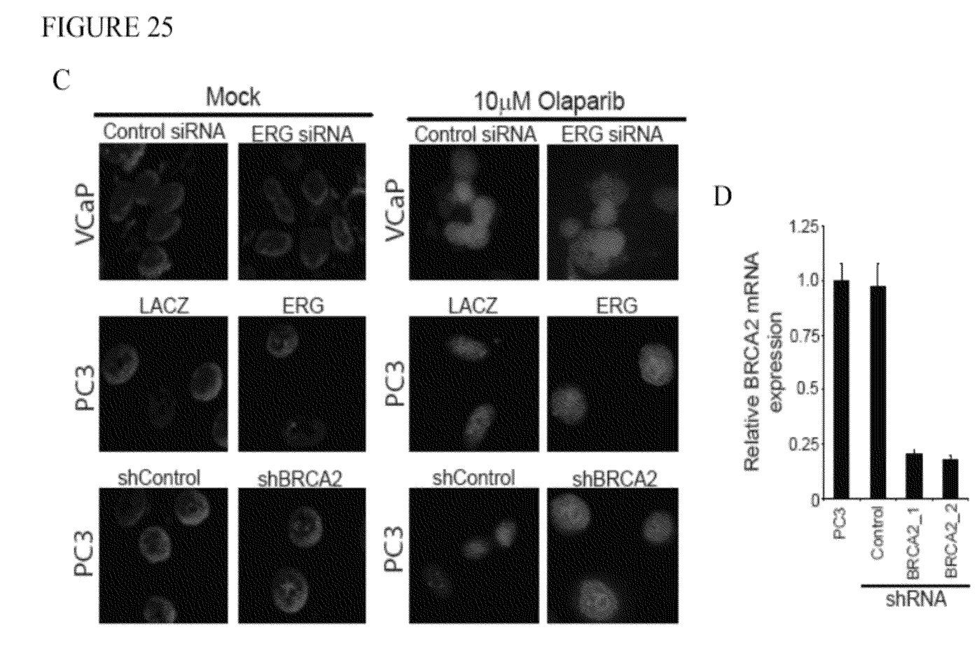

To test the ability of the ETS transcription factors to induce γ-H2A.X foci in other ETS negative prostate cell lines, immortalized benign prostate epithelial cells (RWPE) were infected with either ERG, ETV1 or ETV5 lentiviral expression vectors. Analysis of the stable overexpressing cell lines revealed that all 3 ETS genes were capable of inducing γ-H2A.X foci (FIG. 25a). Likewise, analysis of ERG overexpression in several prostate cell lines revealed that ERG, but not LACZ, induced γ-H2A.X foci in several different genetic backgrounds (FIG. 25b).

While γ-H2A.X foci represent an early mark of DNA damage recognition, 53BP1 is present only in the later stages of repair (Bennett & Harper, Nature structural & molecular biology 15, 20-22 (2008)). The ETS genes induced 53BP1 foci formation in RWPE, VCaP and PrEC cells (FIG. 6a, b and FIG. 25a) indicating that exogenous overexpression of ETS transcription factors induces DNA damage.

After demonstrating that ETS gene overexpression is sufficient to drive the accumulation of markers of DNA double strand breaks, the presence of DNA double strand breaks was confirmed by directly analyzing cellular DNA for fragmentation using the COMET assay. When performed in neutral electrophoresis buffer, the COMET assay measures relative levels of DNA double strand break fragmentation (Fairbairn et al. Mutation research 339, 37-59 (1995)). This is reported as tail moment which assesses the fluorescence intensity in the tail relative to the head while accounting for the relative area of both dipoles. As with the γ-H2A.X and 53BP1 foci formation assays, in PC3 cells, ERG or ETV1 overexpression was sufficient to induce a significantly longer and brighter tail than controls (P<0.01 for both ETS genes). Likewise, ERG siRNA led to a reduction in the average tail moment further suggesting that ETS genes induce DNA double strand breaks (P<0.01) (FIG. 6c, d).

Olaparib Potentiates ETS-Induced DNA Damage

After finding that aberrantly expressed ETS transcription factors drive the accumulation of DNA double strand breaks, it was contemplated that by having a baseline level of DNA damage, ETS positive cancers are specifically susceptible to accumulating DNA damage following inhibition of the interacting DNA repair enzyme PARP1. VCaP cells treated with Olaparib for 48 hours were assayed. Olaparib-treated VCaP cells had a very high level of γ-H2A.X foci (FIG. 25c). By depleting endogenous ERG using siRNA (confirmed in FIG. 11b), it was possible to reverse the gross increase in γ-H2A.X. Similar increases in foci were observed in PC3-ERG cells, but not the control PC3-LACZ (FIG. 25c). Because Olaparib has previously been shown to induce synthetic lethal cell kill by driving DNA double strand breaks in BRCA1/2-deficient tumors, PC3 cells expressing BRCA2 shRNA were generated to compare the ETS-specific effect with a validated positive control of induced γ-H2A.X (Bryant et al. Nature 434, 913-917 (2005); Farmer et al. Nature 434, 917-921 (2005)). As shown in (FIG. 25c), Olaparib caused a similarly dramatic increase in γ-H2A.X staining in the BRCA2 depleted cells. Knockdown efficiency was confirmed by qPCR (FIG. 25d). Because of the high levels of staining observed in the cells, it was not possible to quantify the DNA damage using the foci formation assay.

In order to provide a more accurate quantitative measure of the relative levels of DNA double strand breaks in the different genetic backgrounds, the neutral COMET assay was used to assess relative levels of DNA damage between the control and ERG or ETV1 overexpressing PC3 cells treated with and without Olaparib. ETS overexpression led to an increased tail moment as compared to control cells (P<0.01 for both ETS genes), and Olaparib caused a significantly greater increase in the tail moments of ERG positive cells than controls (P=0.03, two-way ANOVA) (FIG. 6c, d). In conjunction with this observation, ERG siRNA led to a significant reduction in DNA damage following Olaparib, however, it did not block the synergistic increase of DNA damage observed with Olaparib. COMET assays performed in parallel to the ETS assays revealed that Olaparib-treated BRCA2 shRNA PC3 cells had slightly larger tail moments than Olaparib treated ETS overexpressing cells indicating that Olaparib treatment leads to more DNA damage in cells with BRCA2-deficiency than ETS overexpression (P=0.14 for BRCA shRNA_{—}1 and P=0.003 for BRCA shRNA_{—}2, two-way ANOVA). Taken together, this data demonstrates that similar to BRCA2-deficient tumors, ETS positive, but not ETS negative, prostate cancer models are susceptible to PARP inhibition through the increased incidence of DNA double strand breaks (FIG. 6e).

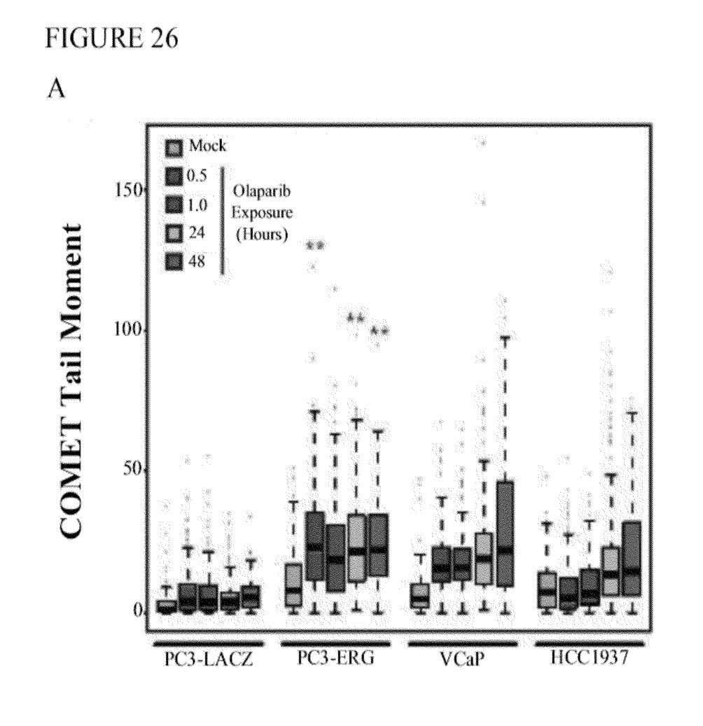

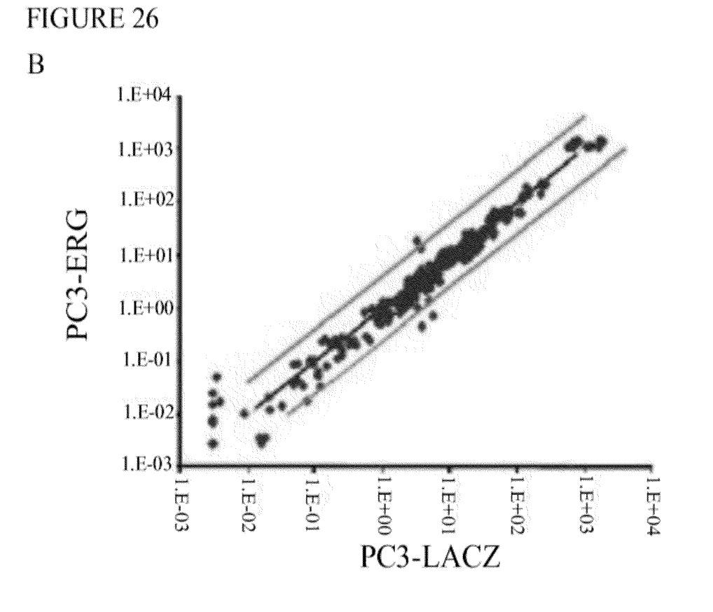

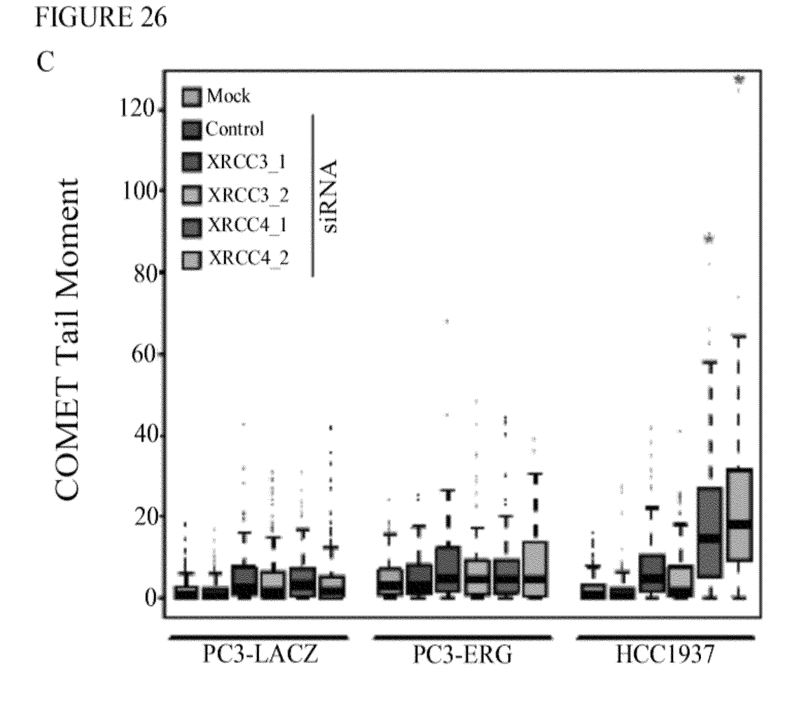

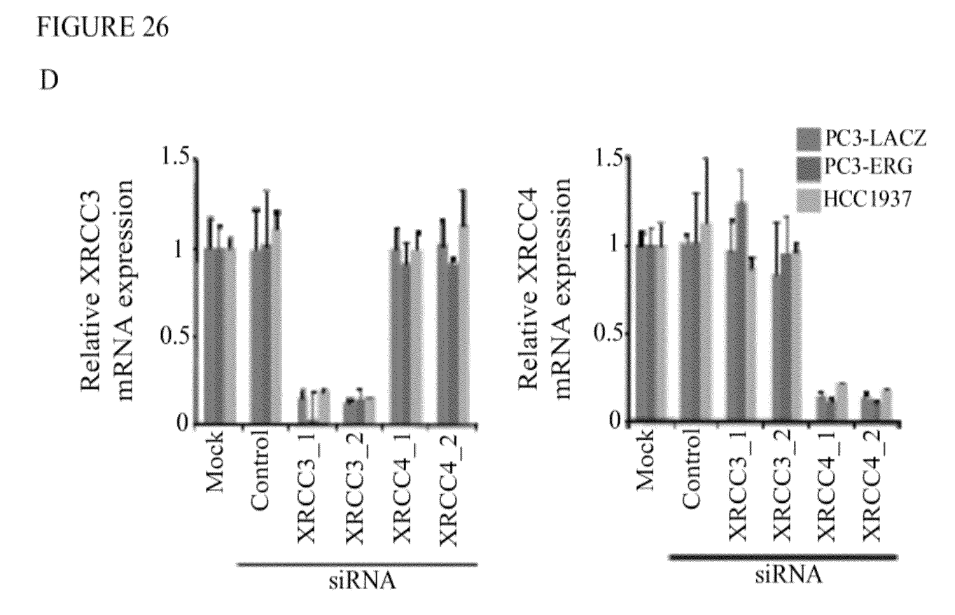

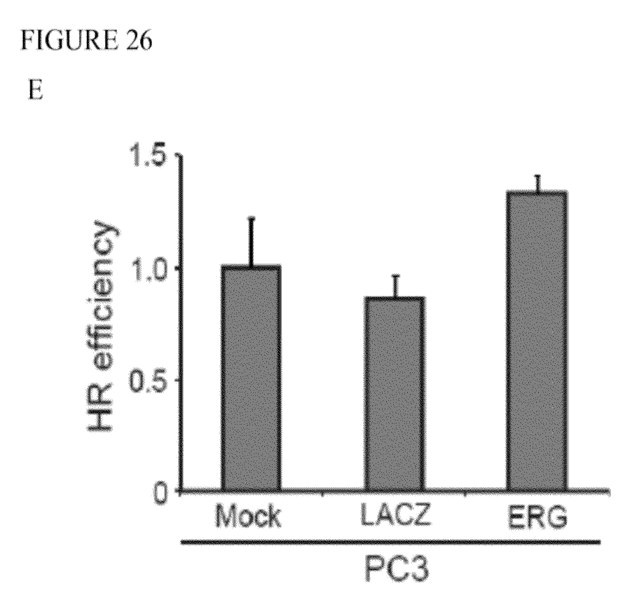

COMET assays were performed after 0.5, 1, 24, and 48 hr exposure to Olaparib. The potentiated DNA damage was observed in PC3-ERG cells relative to PC3-LACZ cells as early as 30 min after treatment (FIG. 26A) (p=0.002 at 30 min, two-way ANOVA). This indicated that ERG-induced potentiation is independent of the genes regulated by PARP1-mediated transcriptional activation. Focused expression analysis of genes involved in DNA-damage repair pathways demonstrated no significant change in any of the repair genes analyzed, indicating that the DNA-damage is independent of changes to ERG-regulated gene expression (FIG. 26B). To analyze the role of repair pathways directly, the postulate that downregulation of a protein critical for the execution of NHEJ pathway such as XRCC4 would lead to a synergistic induction of DNA damage in a homologous recombination (HR)-deficient cell was tested. Treatment of HR-deficient HCC1937 cells with siRNA confirmed a greater increase in DNA damage following XRCC4 knockdown (NHEJ) than by XRCC3 knockdown (HR) one-way ANOVA). In contrast, the synergistic induction of DNA damage following XRCC4 or XRCC3 knockdown was not observed in PC3-ERG cells as compared to PC3-LACZ cells (FIGS. 26C and D). This indicated that ERG overexpression does not completely block either of these double-strand break repair pathways. This was further confirmed by HR-efficiency assays that demonstrated that HR is not significantly altered by ERG overexpression (FIG. 26E).

TABLE 1

| | Protein | Unique | % | Total | |
|---|---|---|---|---|---|
| Protein ID | Probability | Peptides | Coverage | Peptides | Description |
| IPI00005012, IPI00217544, IPI00549287, | 1 | 65 | 64.4 | 573 | ERG 51 kDa protein |
| IPI00788647, IPI00797389, | | | | | |
| IPI00872796 | | | | | |
| IPI00296337 | 1 | 37 | 9.9 | 71 | DNAPK Isoform 1 of DNA-dependent |
| | | | | | protein kinase catalytic subunit |
| IPI00843765, IPI00844215, IPI00871535 | 1 | 36 | 20.4 | 95 | SPTAN1 Isoform 1, 2, or 3 of Spectrin alpha |
| | | | | | chain, brain |
| IPI00644712 | 1 | 18 | 33.5 | 32 | Ku70 ATP-dependent DNA helicase 2 |
| | | | | | subunit 1 |
| IPI00220834 | 1 | 17 | 26 | 46 | Ku80 ATP-dependent DNA helicase 2 |
| | | | | | subunit 2 |
| IPI00479217, IPI00644079, IPI00883857 | 1 | 13 | 19 | 34 | HNRNPU Isoform Short or Long of |
| | | | | | Heterogeneous nuclear ribonucleoprotein |
| | | | | | U, isoform a |
| IPI00013296 | 1 | 9 | 42.8 | 29 | RPS18 40S ribosomal protein S 18 |
| IPI00440493 | 1 | 9 | 21.9 | 22 | ATP5A1 ATP synthase subunit alpha, |
| | | | | | mitochondrial precursor |
| IPI00021439, IPI00021440 | 1 | 9 | 21.6 | 27 | ACTB Actin, cytoplasmic 1 or 2 |
| IPI00301263 | 1 | 9 | 7.1 | 15 | CAD protein |
| IPI00011654, IPI00645452 | 1 | 7 | 55.9 | 15 | TUBB Tubulin beta chain, TUBB Tubulin, |
| | | | | | beta polypeptide |
| IPI00011274, IPI00045498, IPI00845282 | 1 | 7 | 36.5 | 18 | HNRPDL Isoform 1, 2, or 3 of |
| | | | | | Heterogeneous nuclear ribonucleoprotein D- |
| | | | | | like |
| IPI00299573 | 1 | 7 | 30.4 | 43 | RPL7A 60S ribosomal protein L7a |
| IPI00007928 | 1 | 7 | 6.1 | 12 | PRPF8 Pre-mRNA-processing-splicing factor 8 |
| IPI00008530 | 1 | 5 | 31.4 | 8 | RPLP0 60S acidic ribosomal protein P0 |
| IPI00219678 | 1 | 5 | 14.6 | 6 | EIF2S1 Eukaryotic translation initiation |
| | | | | | factor 2 subunit 1 |
| IPI00171903, IPI00383296 | 1 | 5 | 7.5 | 14 | HNRPM Isoform 1 or 2 of Heterogeneous |
| | | | | | nuclear ribonucleoprotein M |
| IPI00216134, IPI00384369 | 1 | 3 | 13.3 | 10 | TPM1 tropomyosin 1 alpha chain isoform |
| | | | | | 7, TPM1 Tropomyosin alpha variant 6 |
| IPI00018971 | 1 | 3 | 8.8 | 6 | TRIM21 52 kDa R o protein |
| IPI00017617 | 0.9999 | 4 | 16.1 | 5 | DDX5 Probable ATP-dependent RNA |
| | | | | | helicase DDX5 |
| IPI00396378, IPI00414696 | 0.9999 | 3 | 12 | 11 | HNRNPA2B1 Isoform A2 or B1 of |
| | | | | | Heterogeneous nuclear ribonucleoproteins |
| | | | | | A2/B1 |
| IPI00016610 | 0.9999 | 2 | 10.1 | 6 | PCBP1 Poly(rC)-binding protein 1 |
| IPI00003865, IPI00007702, IPI00037070, | 0.9998 | 19 | 33.4 | 35 | HSPA8 Isoform 1 or 2 of Heat shock |
| IPI00795040 | | | | | cognate 71 kDa protei HSPA2 Heat shock- |
| | | | | | related 70 kDa protein 2 |
| IPI00215637 | 0.9994 | 3 | 9.7 | 6 | DDX3X ATP-dependent RNA helicase |
| | | | | | DDX3X |
| IPI00012048, IPI00026260, IPI00375531 | 0.9732 | 2 | 14.6 | 2 | NME1; NME2 Nucleoside diphosphate |
| | | | | | kinase A or B, |

TABLE 2

| Gene ID | PARP | DNAPK |
|---|---|---|
| MGA | 0.264462 | 0.468635 |
| TBX15 | 0.270868 | 0.255366 |
| STARD4 | 0.303519 | 0.340856 |
| SUV39H2 | 0.307705 | 0.438169 |
| CD556746 | 0.33708 | 0.384758 |
| KITLG | 0.337534 | 0.387291 |
| NEIL2 | 0.364262 | 0.438382 |
| LAMA1 | 0.380545 | 0.331431 |
| AOC3 | 0.381653 | 0.491479 |
| E2F8 | 0.392452 | 0.447007 |
| HPGD | 0.395736 | 0.425042 |
| CCT8 | 0.401878 | 0.364565 |
| TDRD9 | 0.408429 | 0.401473 |
| BTG3 | 0.415805 | 0.453336 |
| FAM49B | 0.415849 | 0.48009 |
| C2orf14 | 0.420487 | 0.432569 |
| AA631847 | 0.420546 | 0.482415 |
| LOC641467 | 0.4296 | 0.499436 |
| LOC440295 | 0.43172 | 0.30966 |
| SEMA3C | 0.432329 | 0.478795 |
| MTHFD2 | 0.435626 | 0.47799 |
| GNB4 | 0.442082 | 0.270747 |
| ZC3HAV1L | 0.44313 | 0.402285 |
| CDCA7 | 0.443146 | 0.355948 |
| C6orf167 | 0.444557 | 0.352313 |
| C14orf151 | 0.445016 | 0.45962 |
| KCNJ2 | 0.447251 | 0.459773 |
| TTTY15 | 0.447368 | 0.437676 |
| DNMT3A | 0.447503 | 0.374397 |
| RBL1 | 0.451895 | 0.46101 |
| CYCS | 0.453801 | 0.387284 |
| RUNDC2B | 0.454633 | 0.450951 |
| MARS2 | 0.454741 | 0.472233 |
| SEH1L | 0.459784 | 0.425246 |
| ZNF100 | 0.460427 | 0.383418 |
| ARNTL2 | 0.460437 | 0.495687 |
| EZH2 | 0.461723 | 0.394717 |
| ABCB10 | 0.466043 | 0.458422 |
| SFRS1 | 0.4705 | 0.43528 |
| ATAD2 | 0.472069 | 0.367528 |
| KLHL2 | 0.472132 | 0.478127 |
| RIF1 | 0.481235 | 0.477163 |
| A_32_P108420 | 0.481256 | 0.460032 |
| DLEU2 | 0.484133 | 0.348859 |
| FAM29A | 0.484236 | 0.437267 |
| MNS1 | 0.487367 | 0.385271 |
| PFKFB2 | 0.493362 | 0.442273 |
| ARHGAP11A | 0.496593 | 0.498053 |
| DKFZP586B0319 | 0.496614 | 0.350466 |
| CENPK | 0.497873 | 0.456985 |
| CDCA7 | 0.499617 | 0.373323 |

TABLE 3

| Gene ID | PARP AVG | DNAPK avg |
|---|---|---|
| PTPRT | 7.70030415 | 12.38814811 |
| WNK4 | 5.192412457 | 4.938466637 |
| ART3 | 4.974833846 | 7.18782895 |
| SLC7A14 | 4.878305787 | 6.277619055 |
| ZNF536 | 4.772623742 | 4.533890985 |
| FOS | 4.619361735 | 6.830943546 |
| ANKRD38 | 4.586528889 | 3.715707353 |
| PTPRE | 4.220543988 | 7.216677218 |
| FAM135B | 4.17108797 | 4.00391668 |
| SATB1 | 3.853212082 | 5.278828097 |
| CXCL11 | 3.83455567 | 6.043351777 |
| AI674800 | 3.774655874 | 3.359935881 |
| PCDH19 | 3.726251362 | 5.649671458 |
| CLIP3 | 3.592403912 | 4.2223238 |
| SNAP25 | 3.586151379 | 3.459765982 |
| DGKG | 3.560303403 | 3.727748526 |
| OR51E1 | 3.552673916 | 3.643674539 |
| MDK | 3.533255998 | 4.05183402 |
| TFF3 | 3.488900406 | 5.856143397 |
| PGF | 3.414377034 | 4.023247125 |
| WNK4 | 3.393173305 | 3.602688988 |
| BMX | 3.379298932 | 5.014522159 |
| RAB42 | 3.338726755 | 3.816174831 |
| C21orf88 | 3.330321368 | 4.672643797 |
| GUCA2A | 3.322560895 | 5.842426522 |
| GPR120 | 3.243830764 | 3.22160672 |
| SOX30 | 3.233004748 | 3.15963452 |
| KCNB1 | 3.198906582 | 5.300129036 |
| YPEL3 | 3.183857812 | 3.732482033 |
| DIO3 | 3.139929867 | 3.079130444 |
| MYO1G | 3.103429374 | 2.915413073 |
| CD200 | 3.097167617 | 3.119397032 |
| PTCHD2 | 3.058486389 | 2.060747789 |
| C9orf4 | 3.025912731 | 2.821314456 |
| CUEDC1 | 3.01445004 | 3.479976938 |
| BMX | 2.976711988 | 5.688902163 |
| FAM14A | 2.956079502 | 5.947640529 |
| SLFN5 | 2.955286494 | 3.731427691 |
| C1QTNF1 | 2.906643261 | 3.23188441 |
| LOC202134 | 2.870954285 | 3.144101823 |
| FOXN2 | 2.861072924 | 2.841886033 |
| DENND2A | 2.855315895 | 3.299616368 |
| LOC647502 | 2.818920623 | 2.335904836 |
| C12orf51 | 2.811883289 | 2.567690246 |
| NTF5 | 2.809855383 | 2.949001762 |
| Kua-UEV | 2.80509674 | 2.811475466 |
| SLC2A10 | 2.793323573 | 2.81208 |
| NEURL | 2.781187661 | 3.585403804 |
| C20orf74 | 2.776409737 | 2.781188481 |
| TCN2 | 2.769731164 | 2.467443388 |
| KLHL24 | 2.768599437 | 2.707371219 |
| CXCL11 | 2.762173126 | 4.649396729 |
| MRAS | 2.746989189 | 4.892104128 |
| PTPRT | 2.711533306 | 2.955710982 |
| KREMEN1 | 2.708394012 | 2.048622669 |
| ABCC3 | 2.707233358 | 6.689314952 |
| LOC730091 | 2.701996022 | 5.8574316 |
| DDX17 | 2.696100445 | 2.196304099 |
| NAP5 | 2.695599445 | 3.433230297 |
| HSD17B2 | 2.687121255 | 5.041617614 |
| GRIA2 | 2.686483176 | 2.947342554 |
| ARRDC3 | 2.681252648 | 2.053635972 |
| KLC4 | 2.680830825 | 2.930158932 |
| TMEM30B | 2.668044825 | 2.090521952 |
| GRK4 | 2.662371456 | 4.490892784 |
| TMEM169 | 2.650213657 | 5.55493138 |
| KIAA1853 | 2.649369301 | 3.276867201 |
| CIB2 | 2.644661881 | 3.393110914 |
| HRNBP3 | 2.641760737 | 2.060505756 |
| PPFIA2 | 2.632260965 | 4.241246332 |
| ANGPTL2 | 2.629201451 | 2.651022223 |
| KIAA0152 | 2.628899789 | 2.888697361 |
| RASSF4 | 2.627694026 | 3.321657122 |
| TPCN1 | 2.627080083 | 2.293608891 |
| SLC9A3 | 2.606581088 | 2.278199224 |
| IGFBP7 | 2.605145011 | 5.418048859 |
| RC3H1 | 2.602608321 | 2.679790886 |
| SLC4A4 | 2.596961899 | 2.790356882 |
| FSTL4 | 2.586459217 | 3.424040811 |
| KCNC2 | 2.57353773 | 3.1137801 |
| C15orf48 | 2.569924713 | 3.682070272 |
| TLN2 | 2.566907798 | 2.402090983 |
| KIAA1946 | 2.558402003 | 2.212658711 |
| ABTB1 | 2.556338109 | 2.525222338 |
| LIX1 | 2.554646943 | 4.473526016 |
| KIAA0319L | 2.540977166 | 2.631219205 |
| NY-REN-7 | 2.514299984 | 2.71642234 |
| RPL10 | 2.508924582 | 2.39084502 |
| AMT | 2.504305276 | 2.005439682 |
| LOXL4 | 2.498175214 | 2.881338949 |
| SHC2 | 2.493785892 | 2.960059429 |
| SV2B | 2.490237445 | 4.465935075 |
| HAB1 | 2.486545766 | 2.599558782 |
| IGFBP6 | 2.48608148 | 2.82969242 |
| LGICZ1 | 2.478803133 | 2.404460254 |
| HIP1 | 2.475583982 | 2.248710783 |
| NAV1 | 2.466656695 | 2.462255496 |
| ABTB1 | 2.458763297 | 2.44099941 |
| HADHA | 2.457656294 | 2.19746695 |
| CCDC3 | 2.45473911 | 2.729498938 |
| PCMTD1 | 2.454075598 | 2.433000175 |
| C20orf102 | 2.441737478 | 3.219162291 |
| KIAA1212 | 2.440569185 | 3.286796191 |
| HOXC8 | 2.440309748 | 2.588697282 |
| SNIP | 2.439988394 | 2.865091543 |
| FOXO4 | 2.438701475 | 2.836579324 |
| AMOTL1 | 2.436568985 | 2.120843585 |
| EXT1 | 2.435215249 | 2.20794906 |
| LOC642852 | 2.434432243 | 2.70856026 |
| NEFM | 2.434016648 | 2.989242813 |
| FCGRT | 2.431672727 | 2.642880078 |
| SLC24A3 | 2.422565647 | 4.01825443 |
| NAP5 | 2.412047823 | 3.594028453 |
| CLSTN3 | 2.407236865 | 2.082550975 |
| HRASLS3 | 2.399853986 | 2.533495743 |
| GRIA2 | 2.397383796 | 3.652310525 |
| MALAT1 | 2.396115888 | 3.19757031 |
| LPP | 2.392200462 | 2.94913643 |
| PHF2 | 2.392136575 | 2.282561209 |
| EIF2AK2 | 2.389472678 | 2.027073909 |
| CTGF | 2.387586467 | 3.991631002 |
| SH3KBP1 | 2.38239472 | 3.621110522 |
| TSPAN1 | 2.376540238 | 2.396320761 |
| NLRP1 | 2.364919283 | 4.206623285 |
| SCN8A | 2.355579119 | 2.030031227 |
| MSRB3 | 2.351674107 | 2.447156244 |
| PRSS2 | 2.348646256 | 3.12927397 |
| ERC1 | 2.32685231 | 2.197182341 |
| SNIP | 2.328384471 | 3.267025165 |
| MYST4 | 2.325062115 | 2.327690499 |
| MYO5A | 2.32007226 | 2.196405936 |
| RYR1 | 2.318345511 | 2.732240039 |
| PBX1 | 2.318003505 | 3.0127011 |
| PCMTD1 | 2.317459794 | 2.280181639 |
| VANGL1 | 2.316719497 | 2.512150541 |
| CASC4 | 2.316536952 | 2.181142984 |
| MYO1G | 2.316142321 | 2.851614965 |
| Kua-UEV | 2.31330771 | 2.366885725 |
| KIF12 | 2.307215533 | 2.408909217 |
| MAP3K10 | 2.303608517 | 2.519000903 |
| JAM3 | 2.303427201 | 3.09488908 |
| VTN | 2.300978813 | 3.370689768 |
| TOX | 2.300881316 | 2.544735637 |
| KCNK13 | 2.295870346 | 2.555604765 |
| HSPG2 | 2.284879564 | 2.800786972 |
| PLEKHB1 | 2.280363904 | 3.580786601 |
| ENO2 | 2.278387098 | 2.234038824 |
| CPM | 2.276534268 | 3.278718155 |
| C21orf34 | 2.275673738 | 2.787111113 |
| MIER1 | 2.274298017 | 2.093847974 |
| CCDC125 | 2.270932195 | 2.092521223 |
| SCN4B | 2.26277934 | 2.94343761 |
| CDH22 | 2.258925812 | 4.637843664 |
| ITM2A | 2.252059264 | 3.074315034 |
| LOC157860 | 2.25077237 | 3.156891246 |
| SERPINA1 | 2.250484807 | 2.379128952 |
| TMEM45A | 2.245966984 | 3.204248658 |
| CCDC113 | 2.245396216 | 3.073031202 |
| ADPRH | 2.245239526 | 2.279578032 |
| C20orf23 | 2.242990953 | 2.438202896 |
| CACNG6 | 2.239689235 | 3.373792367 |
| S100A6 | 2.239337306 | 2.926295924 |
| PRSS2 | 2.239242512 | 2.942914902 |
| MB | 2.236818075 | 2.777079308 |
| TMC8 | 2.23542049 | 2.678840856 |
| PDE6D | 2.230404847 | 2.450824763 |
| PIK3AP1 | 2.228892026 | 2.315956061 |
| VASP | 2.228651624 | 2.476634349 |
| SLC13A3 | 2.22850027 | 3.555793437 |
| COL6A2 | 2.227050302 | 2.012069404 |
| FLOT2 | 2.226659368 | 2.347385797 |
| DUSP6 | 2.224398559 | 3.60111194 |
| SVOPL | 2.222722559 | 4.673269409 |
| ERG | 2.222269589 | 2.298243108 |
| NEURL | 2.222051594 | 2.792572767 |
| KCNJ3 | 2.218303855 | 3.460455649 |
| ZNF467 | 2.214526461 | 2.136862893 |
| DLGAP4 | 2.214315782 | 2.284322878 |
| LOC253264 | 2.213666379 | 2.802255771 |
| ACPP | 2.21299316 | 2.784264001 |
| LY6G5C | 2.212215363 | 2.257931201 |
| FBXO11 | 2.20466552 | 2.42517869 |
| ACPP | 2.203872836 | 2.205342388 |
| LOC388242 | 2.202646103 | 2.81284309 |
| YPEL2 | 2.19815348 | 3.1780016 |
| PGPEP1 | 2.19789844 | 3.064487812 |
| GOLPH4 | 2.197794144 | 2.340983023 |
| PHF2 | 2.194053426 | 2.164856842 |
| NACAD | 2.192685965 | 3.886551785 |
| COL14A1 | 2.192001928 | 4.351479533 |
| RGPD5 | 2.191393806 | 2.368557763 |
| FLJ31356 | 2.190107395 | 3.284793275 |
| ACTA2 | 2.188752369 | 3.423238856 |
| LPIN2 | 2.188005608 | 2.516460636 |
| ZNF658 | 2.184714845 | 2.219632838 |
| TPCN1 | 2.178778226 | 2.496042717 |
| KCNK3 | 2.177351722 | 2.299537884 |
| GPR120 | 2.173726606 | 2.266339444 |
| NOV | 2.172924095 | 2.545149432 |
| RALB | 2.172864079 | 2.15887803 |
| LIG3 | 2.165928404 | 2.17568689 |
| SELV | 2.165578513 | 2.884150641 |
| MMP7 | 2.162322561 | 3.664149548 |
| WDR78 | 2.162308678 | 2.199790444 |
| SLC18A1 | 2.162183562 | 2.155280986 |
| SHE | 2.162113469 | 2.189933812 |
| OTUD5 | 2.160873192 | 2.016093783 |
| GPR161 | 2.160175377 | 2.048701761 |
| ENC1 | 2.156226966 | 2.653489225 |
| EMID1 | 2.153494747 | 2.611273004 |
| H6PD | 2.150517519 | 2.184824285 |
| WASF2 | 2.148444617 | 2.094248538 |
| TSPAN2 | 2.14679634 | 2.74722941 |
| NPAL2 | 2.14594161 | 2.371281823 |
| PSD3 | 2.144400024 | 2.111979851 |
| BTC | 2.142341323 | 3.422432613 |
| SPTBN4 | 2.141977197 | 3.259752072 |
| SLC43A2 | 2.137531668 | 2.483782651 |
| COPZ2 | 2.137326477 | 2.456759912 |
| CCDC88 | 2.136327761 | 2.497885114 |
| CDH7 | 2.131871803 | 2.494388665 |
| FBXO27 | 2.127484462 | 2.080622476 |
| MEA1 | 2.125922784 | 2.083684382 |
| KCTD16 | 2.125458545 | 2.473628683 |
| GPR143 | 2.123931251 | 2.203760349 |
| NMNAT2 | 2.119644166 | 2.065755117 |
| ZDHHC17 | 2.116381602 | 2.173788293 |
| ZNF618 | 2.115424875 | 2.226712907 |
| SLC35F3 | 2.112739731 | 2.631890729 |
| LIMD2 | 2.110467651 | 2.442788665 |
| SVOP | 2.109529156 | 2.832215131 |
| BACE1 | 2.107283138 | 2.611984337 |
| ERC2 | 2.102506647 | 2.928585332 |
| CHRDL2 | 2.102148378 | 2.286754203 |
| ABLIM3 | 2.101433028 | 2.255025509 |
| APCDD1 | 2.099848086 | 2.75921464 |
| TMEM16B | 2.099183718 | 3.165944622 |
| NPR2 | 2.098483672 | 2.573269113 |
| PALLD | 2.091088474 | 3.41721504 |
| KIAA1913 | 2.088103402 | 2.471119087 |
| BCMO1 | 2.086638241 | 2.291138156 |
| NEU4 | 2.085765676 | 2.967859266 |
| SORBS2 | 2.085168811 | 2.307658986 |
| APOD | 2.084253506 | 2.376549379 |
| PTPRE | 2.081370449 | 4.041988731 |
| GDAP1L1 | 2.079600324 | 2.730600445 |
| TNFSF15 | 2.074546468 | 2.344583658 |
| RAB3C | 2.072999378 | 3.034200049 |
| RAPGEF1 | 2.068392895 | 2.219144822 |
| KIAA0152 | 2.066501168 | 2.195504182 |
| ZNF776 | 2.064925357 | 2.257451007 |
| RENBP | 2.063193717 | 2.769437985 |
| LOC439914 | 2.056251994 | 2.929069394 |
| FLOT2 | 2.054778173 | 2.178927051 |
| MCAM | 2.054439946 | 2.392804947 |
| B3GALT4 | 2.050369903 | 3.37957417 |
| B3GALT4 | 2.048325408 | 3.184313949 |
| LIM2 | 2.047933177 | 2.229626062 |
| CRABP2 | 2.044886286 | 2.288684191 |
| PRRC1 | 2.044645188 | 2.103451335 |
| SCARNA17 | 2.044008637 | 2.370437108 |
| CDRT4 | 2.043086688 | 4.742016905 |
| ACIN1 | 2.039520485 | 2.01135997 |
| CCDC88 | 2.035788825 | 2.555526079 |
| RBP1 | 2.035457249 | 2.522963799 |
| ZNF704 | 2.034631367 | 2.214568178 |
| ChGn | 2.031489883 | 2.27215819 |
| FNDC4 | 2.028600829 | 2.22360749 |
| C20orf194 | 2.027046038 | 2.691134303 |
| TNNC2 | 2.026078803 | 2.361715798 |
| AMT | 2.02564435 | 2.253505214 |
| UFM1 | 2.025116237 | 2.134596357 |
| LOXL4 | 2.020069462 | 2.011686735 |
| CXCL9 | 2.019575554 | 3.668812975 |
| SFTPD | 2.019476688 | 3.127006415 |
| HIP1 | 2.01943097 | 2.521317756 |
| SMARCD3 | 2.015494327 | 2.622630043 |
| NDRG2 | 2.013153799 | 2.349000533 |
| PRSS3 | 2.011519053 | 2.511323755 |
| RNF152 | 2.008667575 | 2.952687095 |
| MAP1LC3A | 2.00822473 | 2.318817285 |
| ICAM1 | 2.003071273 | 2.80829072 |

TABLE 4

| Assay | Gene/Region | Sequence | Sequence 5′ to 3′ |
|---|---|---|---|
| Expression qPCR | DNALIG4 | NM_001098268 | AGCTGGGATTCTCTGGTTCA |
| Expression qPCR | DNALIG4 | NM_001098268 | TGCAAAAGGAACGTGAGATG |
| Expression qPCR | PARP1 | NM_001618 | GCGTGAAGGCGAATGCCAGC |
| Expression qPCR | PARP1 | NM_001618 | TGTAGCCTGTCACGGGCGCT |
| Expression qPCR | XRCC4 | NM_022406.2 | CCTCAGGAGAATCAGCTTCAA |
| Expression qPCR | XRCC4 | NM_022406.2 | GTCTTCTGGGCTGCTGTTTC |
| Expression qPCR | ATM | NM_000051.3 | GCTGTGAGAAAACCATGGAAG |
| Expression qPCR | ATM | NM_000051.3 | AGTTTCATCTTCCGGCCTCT |
| Expression qPCR | ATR | NM_001184.3 | AAGCGCCACTGAATGAAACT |
| Expression qPCR | ATR | NM_001184.3 | AACGGCAGTCCTGTCACTCT |
| Expression qPCR | DNA-PKcs | NM_001081640.1 | TGCCAATCCAGCAGTCATTA |
| Expression qPCR | DNA-PKcs | NM_001081640.1 | TGAAAGCCCACTCTCTGGTT |
| Expression qPCR | ERG | NM_004449.3 | CGCAGAGTTATCGTGCCAGCAGAT |
| Expression qPCR | ERG | NM_004449.3 | CCATATTCTTTCACCGCCCACTCC |
| Expression qPCR | ETV1 | NM_004956.3 | ATAGCAGCTACCCCATGGAC |
| Expression qPCR | ETV1 | NM_004956.3 | TCAGACATCTGGCGTTGGTA |
| Expression qPCR | EZH2 | NM_004456.3 | AGAATGGAAACAGCGAAGGA |
| Expression qPCR | EZH2 | NM_004456.3 | TCAATGAAAGTACCATCCTGATCT |
| Expression qPCR | BRCA2 | NM_000059.3 | GCCGTACACTGCTCAAATCA |
| Expression qPCR | BRCA2 | NM_000059.3 | TTTGAAGTCATCTGGGCTGA |
| Expression qPCR | DNMT3a | NM_022552.3 | CACCGGCCATACGGTGGAGC |
| Expression qPCR | DNMT3a | NM_022552.3 | TCGTGGTCTTTGGAGGCGAGAGT |
| Expression qPCR | ZNF100 | NM_173531.3 | TGGAGGAGTGGCAATGCCTGGA |
| Expression qPCR | ZNF100 | NM_173531.3 | TGGCTTAGTGAGAGCAATACCTGCCAA |
| Expression qPCR | PBX1 | NM_002585.2 | AGCCGGACCAGGCCCATCTC |
| Expression qPCR | PBX1 | NM_002585.2 | CTTCCGCCGCGCATCCAGAA |
| Expression qPCR | ZNF618 | NM_133374.2 | CTCCTCCCGCACGGACCCAT |
| Expression qPCR | ZNF618 | NM_133374.2 | TTCAAGCGCTCCCTGCTCGC |
| Expression qPCR | KLK3 (PSA) | NM_001648.2 | GAGCACCCCTATCAACCCCCTATT |
| Expression qPCR | KLK3 (PSA) | NM_001648.2 | AGCAACCCTGGACCTCACACCTAA |
| Expression qPCR | β-Actin | NM_001101.3 | CGCGAGAAGATGACCCAGAT |
| Expression qPCR | β-Actin | NM_001101.3 | GAGTCCATCACGATGCCAGT |
| ChIP-qPCR | PLA1A | Promoter (NM_134102) | TGGCCACCCAGAGATGCAGGA |
| ChIP-qPCR | PLA1A | Promoter (NM_134102) | ACACACTGTCCCTCTTTGAGCCA |
| ChIP-qPCR | TMPRSS2 | Enhancer (NM_005656) | TGGAGCTAGTGCTGCATGTC |
| ChIP-qPCR | TMPRSS2 | Enhancer (NM_0D5656) | CTGCCTTGCTGTGTGAAAAA |
| ChIP-qPCR | FKBP5 | Enhancer (NM_001145775) | GGTTCCTGGGCAGGAGTAAG |
| ChIP-qPCR | FKBP5 | Enhancer (NM_001145775) | AACGTGGATCCCACACTCTC |
| ChIP-qPCR | KLK3 (PSA) | Enhancer (NM_001030050) | CCTAGATGAAGTCTCCATGAGCTACA |
| ChIP-qPCR | KLK3 (PSA) | Enhancer (NM_001030050) | GGGAGGGAGAGCTAGCACTTG |

TABLE 5
siRNA sequences

| siRNA | Sequence |
|---|---|
| PARP1 siRNA 1 | GUUCUUAGCGCACAUCUUG |
| PARP1 siRNA 2 | CCAAUAGGCUUAAUCCUGU |
| DNA-Pkcs siRNA 1 | GAGCAUCACUUGCCUUUAA |
| DNA-PKcs siRNA 2 | AGAUAGAGCUGCUAAAUGU |

### Example 2

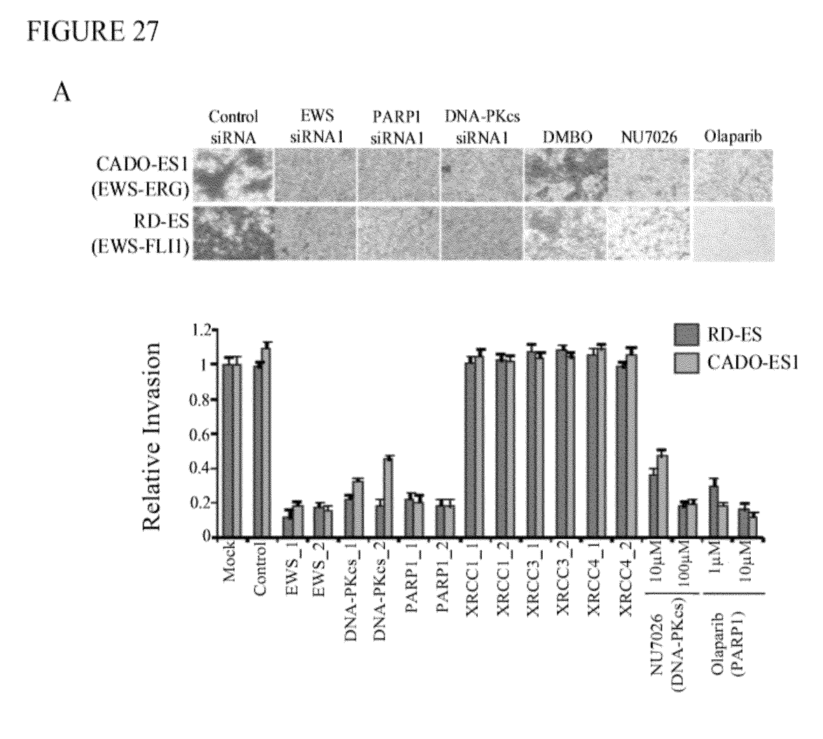

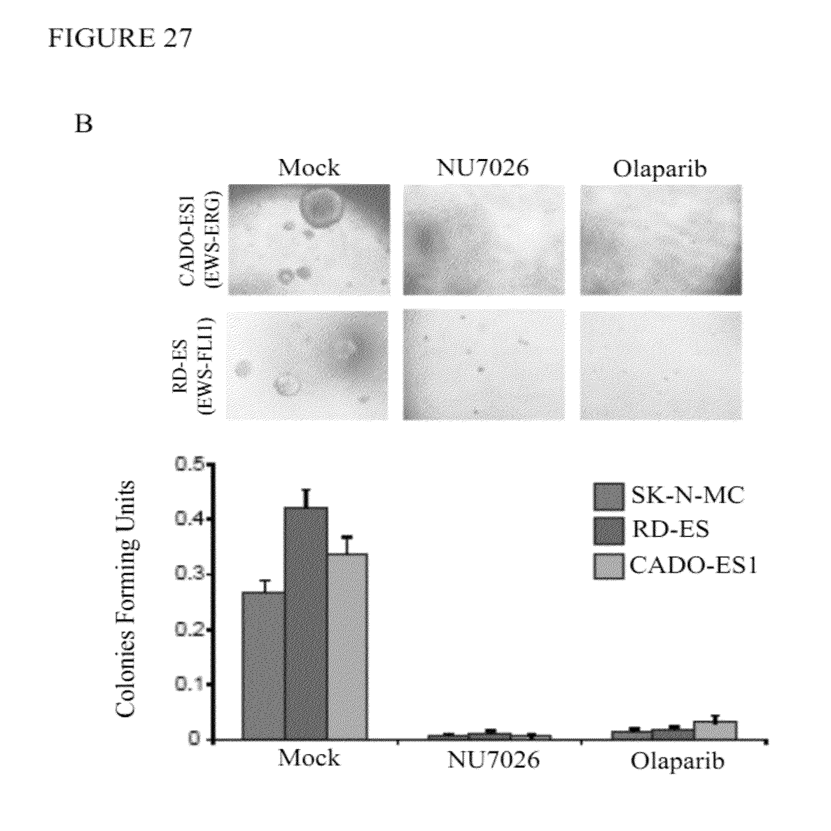

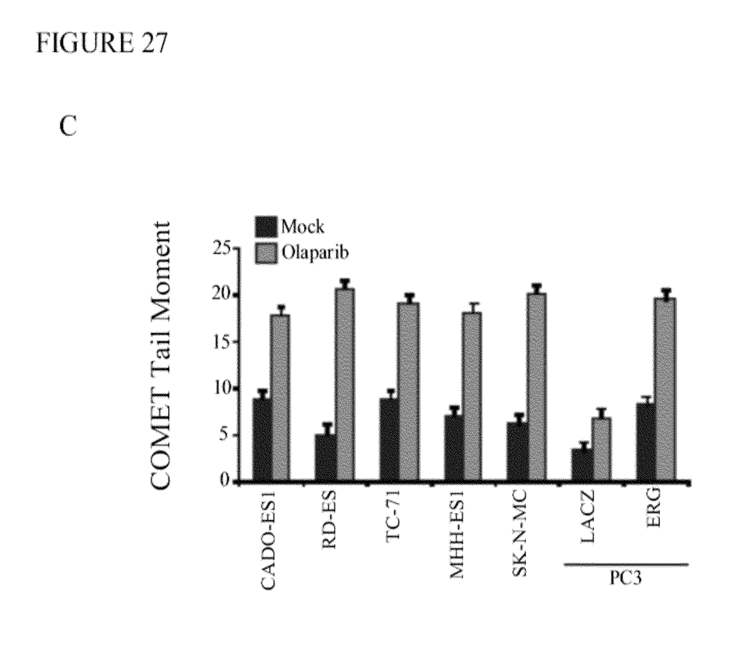

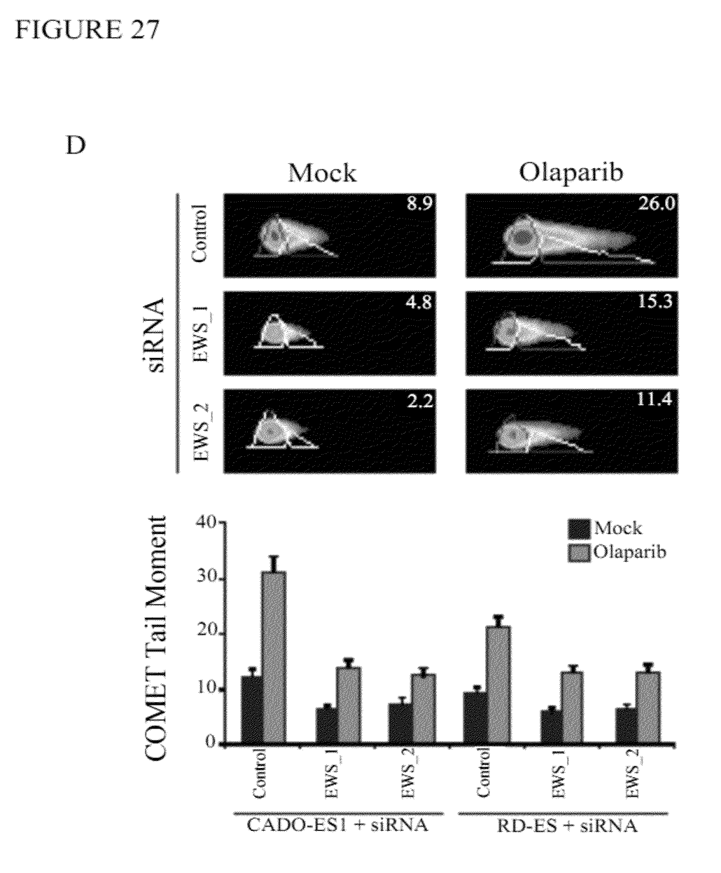

The effect of PARP1 inhibitors on Ewing's sarcoma cell lines was assayed. CADO-ES1 or RD-ES1 cells were treated with siRNA for 48 hours. Cells were then harvested and plated in a transwell migration assay. Forty eight hours later, cells were stained with crystal violet and the total number or invaded cells was quantified. Soft agar colony formation was performed on cells treated with or without Olaparib. Neutral COMET assays were performed on a panel of Ewing's Sarcoma cell lines. PC3 was used as an ETS negative control and PC3-ERG for comparison to an ETS positive model. Cells were treated with or without 10 μM Olaparib for 48 hours. D) As in C, except cells were pre-treated with siRNA for 48 hours. All experiments were run in triplicate. Error bars are standard error of the mean. Results are shown in FIG. 27. Ewing's sarcoma cell lines were shown to be sensitive to PARP1

### Example 3

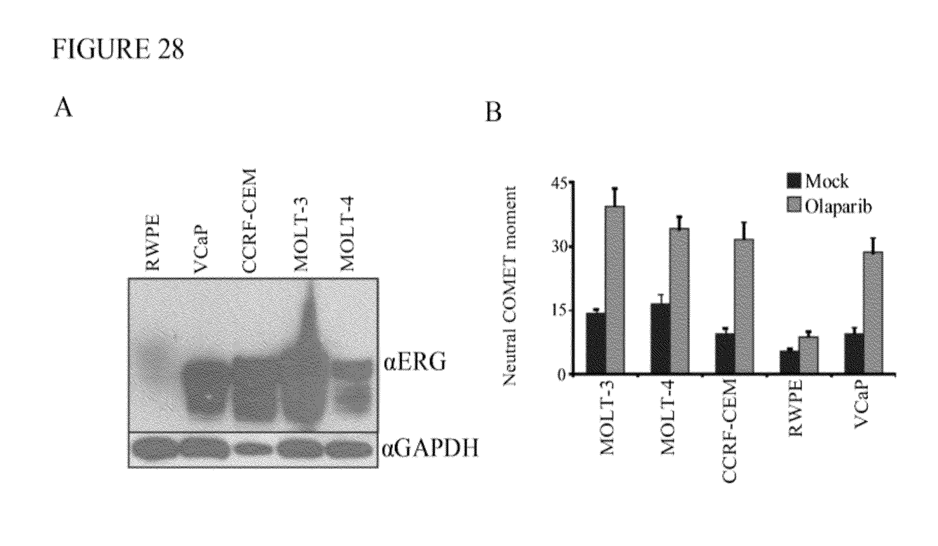

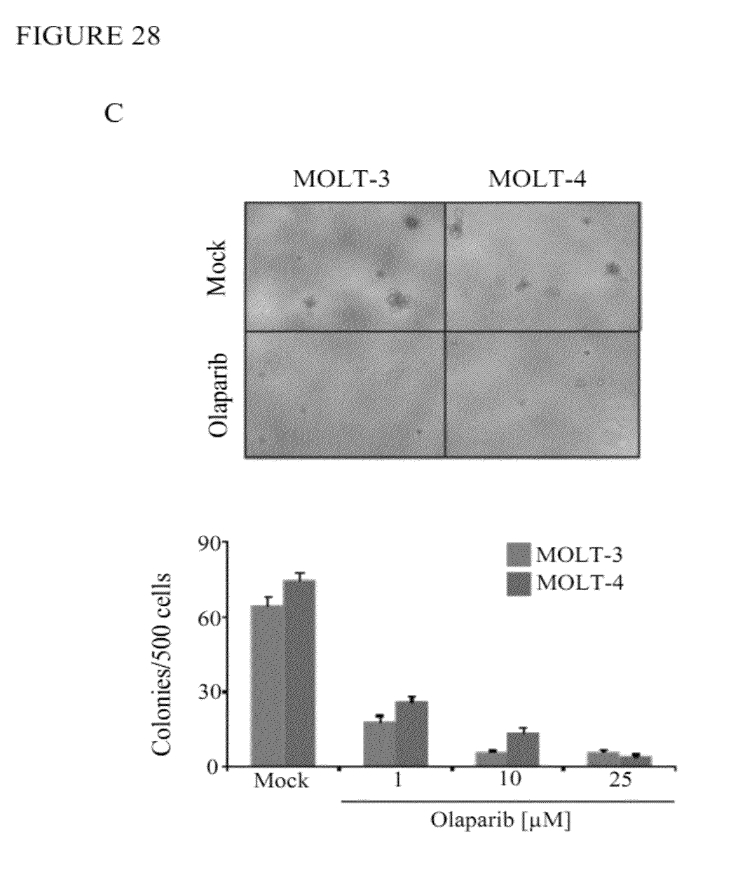

The effect of PARP1 inhibitors on T-ALL cell lines was assayed using Western blot analysis of several T-ALL cell lines comparing total ERG expression levels. In addition, neutral COMET assays were performed on a panel of T-ALL cell lines. RWPE was used as an ETS negative control and VCaP cells were used for comparison to an ETS positive model. Cells were treated with or without 10 μM Olaparib for 48 hours as indicated. Soft agar colony formation was performed on cells treated with or without Olaparib as indicated. All experiments were run in triplicate. Error bars are standard error of the mean. Results are shown in FIG. 28 and indicated that T-ALL cell lines are sensitive to PARP1 inhibition.

### Example 4

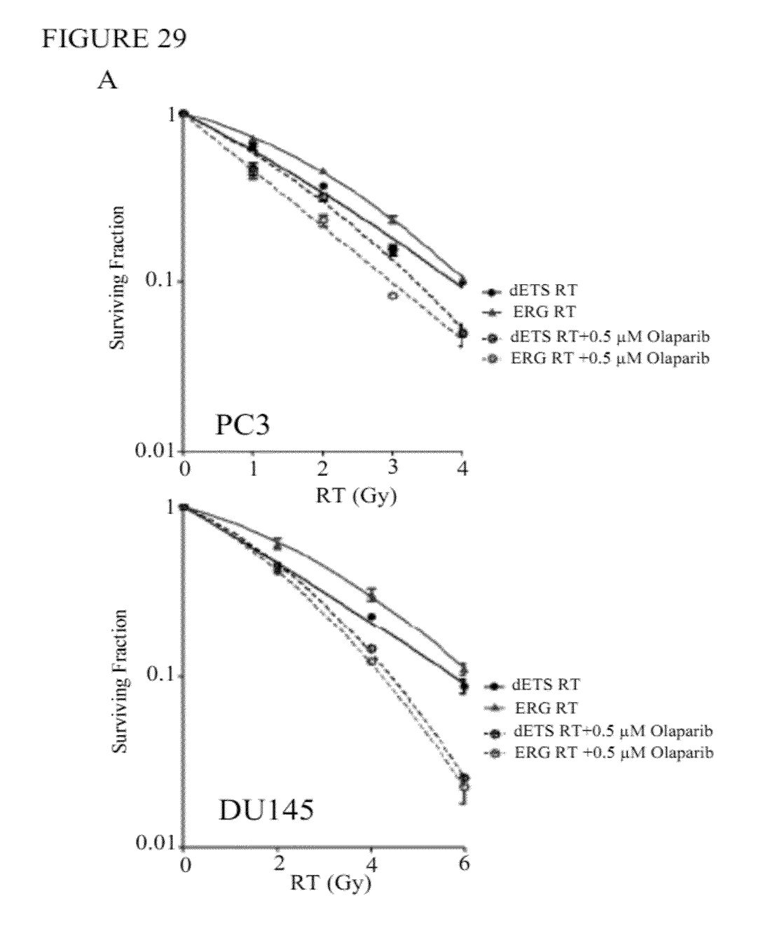

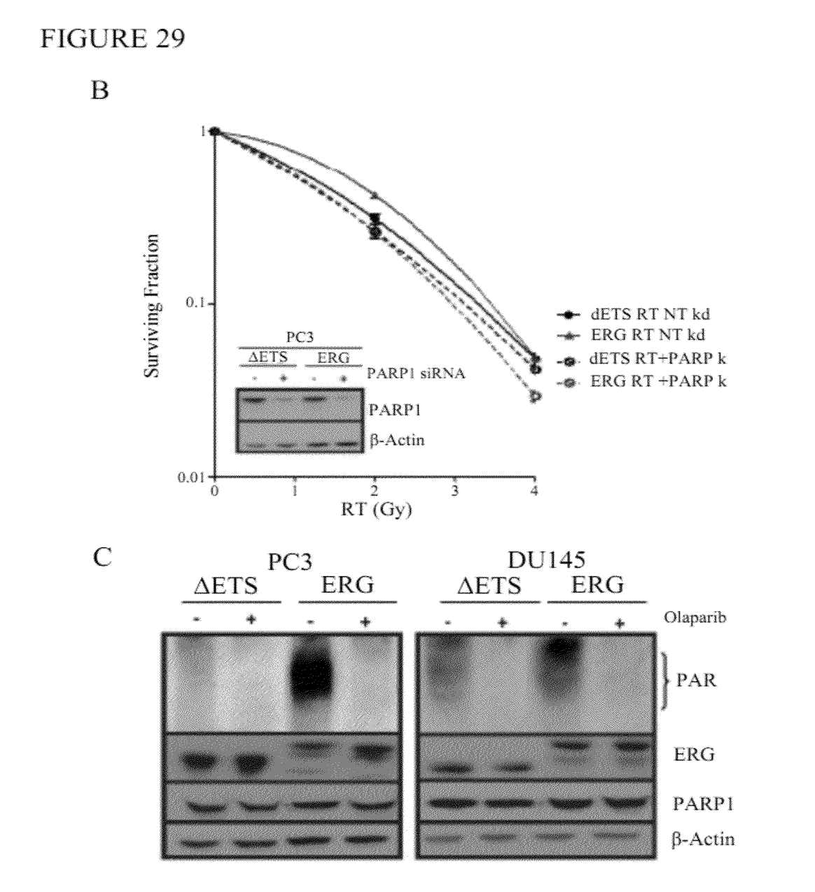

The effect of ETS overexpression on radioresistance was assayed. Isogenic PC3 or DU145 models overexpressing either T2:ERG or ERG with a deleted ETS domain were pre-treated with or without 10 μM Olaparib and then treated with different doses of radiation as indicated. Colony formation was assessed. Experiments were also performed in which cells were pre-treated with either PARP1 or control siRNA prior to radiation. Western blot analysis was used to assess PARP knockdown efficiency in the inset. Total Poly(ADP-ribose) (or PAR) levels were assessed by Western blot analysis from total cell lysates. All experiments were run in triplicate. Error bars are standard error of the mean. Results are shown in FIG. 29 and demonstrate that PARPi blocks ERG induced radiation resistance in PC3 and DU145 cells.

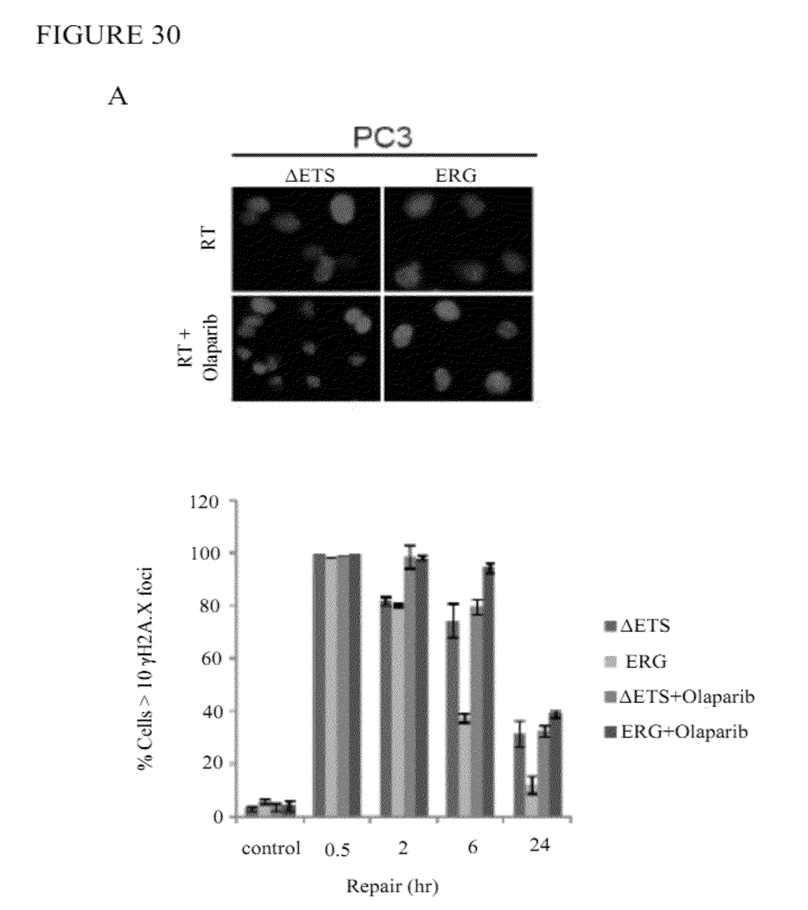

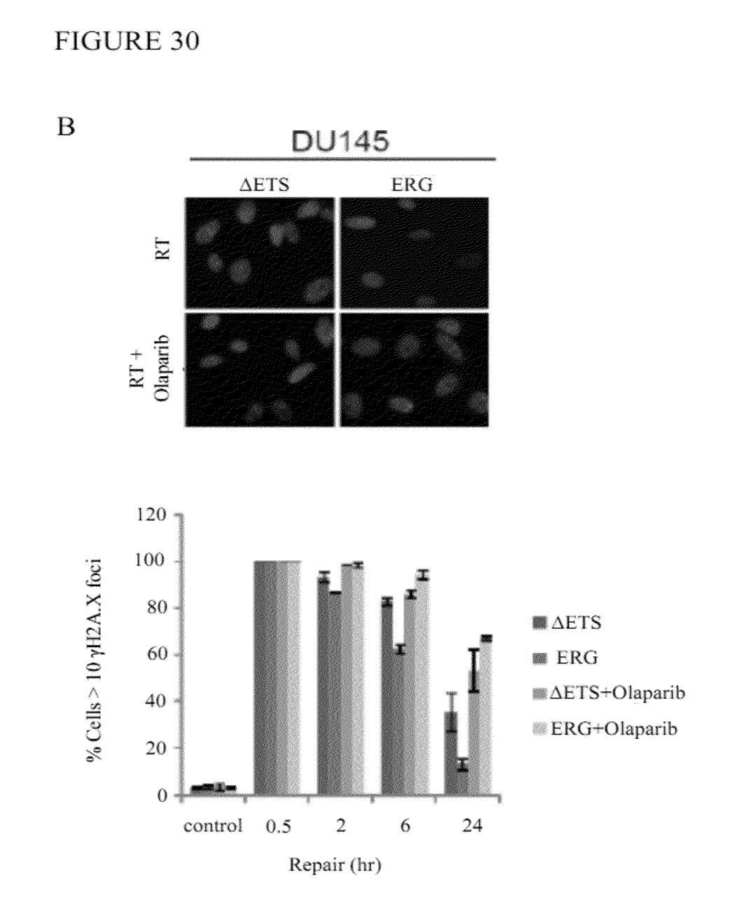

The effect of ETS overexpression on repair of DNA double strand breaks was assayed. Isogenic PC3 and DU145 models, respectively, overexpressing either T2:ERG or ERG with a deleted ETS domain were pre-treated with or without 10 μM Olaparib and then treated with radiation. Immunofluorescence staining of γH2A.X foci was used to assess total levels of DNA damage and repair rate. The number of cells with >10 γH2A.X foci were quantified at each time point as indicated. Representative photomicrographs were taken 24 hours after treatment using an Olympus confocal microscope with a 100× Oil objective. All experiments were run in triplicate. Error bars are standard error of the mean. Results are shown in FIG. 30 and demonstrate that ERG-enhances DNA DSB damage repair, which is blocked by Olaparib.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method of inhibiting a biological activity of PARP1 in a cell, wherein said cell comprises a gene fusion of an ETS family member gene, comprising contacting said cell with a molecule that inhibits at least one biological activity of PARP1.

2. The method of claim 1, wherein said ETS family member gene is ERG.

3. The method of claim 1, wherein said molecule is an siRNA.

4. The method of claim 1, wherein said molecule is a small molecule.

5. The method of claim 1, wherein said small molecule is selected from the group consisting of 8-hydroxy-2-methylquinazolinone (NU1025), AZD2281 (Olaparib), BSI-201, ABT-888, AG014699, CEP 9722, MK 4827, LT-673 and 3-aminobenzamide.

6. The method of claim 5, wherein said small molecule is Olaparib.

7. The method of claim 1, wherein said cell is a cancer cell.

8. The method of claim 7, wherein said cancer cell is a prostate cancer cell.

9. The method of claim 1, wherein said cell is in vivo.

10. The method of claim 9, wherein said cell is in an animal.

11. The method of claim 10, wherein said animal is a human.

12. The method of claim 1, wherein said cell is ex vivo.

13. The method of claim 1, wherein said ETS family member gene is fused to an androgen regulated gene.

14. The method of claim 13, wherein said ETS family member gene is ERG and said androgen regulated gene is TMPRSS2.

15. The method of claim 1, wherein said biological activity is selected from the group consisting of growth of said cell and invasion of said cell.

16. The method of claim 1, further comprising the step of administering a known chemotherapeutic agent to said cell.

17. A method, comprising:
a) assaying a cancer sample from a subject for the presence or absence of a gene fusion comprising an ETS family member; and
b) administering a PARP1 inhibitor to said subject when said cancer sample has the presence of said gene fusion comprising an ETS family member.

18. The method of claim 17, wherein said PARP1 inhibitor inhibits at least one biological activity of PARP1.

19. The method of claim 18, wherein said biological activity is selected from the group consisting of growth of said cell and invasion of said cell.

20. The method of claim 17, wherein said subject has been diagnosed with prostate cancer.

21. The method of claim 16, further comprising the step of administering a known chemotherapeutic agent to said subject.


SEQUENCE LISTING (99 sequences)

SEQ ID NO: 1 - DNA, 20 bp, Artificial Sequence
  Synthetic
ccccattgacttgcctagaa

SEQ ID NO: 2 - DNA, 20 bp, Artificial Sequence
  Synthetic
ggcttttaggggatcttcca

SEQ ID NO: 3 - protein, 15 aa, Homo sapiens
VVSEDFLQDVSASTK

SEQ ID NO: 4 - protein, 13 aa, Homo sapiens
QQVPSGESAILDR

SEQ ID NO: 5 - DNA, 20 bp, Artificial Sequence
  Synthetic
agctgggattctctggttca

SEQ ID NO: 6 - DNA, 20 bp, Artificial Sequence
  Synthetic
tgcaaaaggaacgtgagatg

SEQ ID NO: 7 - DNA, 20 bp, Artificial Sequence
  Synthetic
gcgtgaaggcgaatgccagc

SEQ ID NO: 8 - DNA, 20 bp, Artificial Sequence
  Synthetic
tgtagcctgtcacgggcgct

SEQ ID NO: 9 - DNA, 21 bp, Artificial Sequence
  Synthetic
cctcaggagaatcagcttcaa

SEQ ID NO: 10 - DNA, 20 bp, Artificial Sequence
  Synthetic
gtcttctgggctgctgtttc

SEQ ID NO: 11 - DNA, 21 bp, Artificial Sequence
  Synthetic
gctgtgagaaaaccatggaag

SEQ ID NO: 12 - DNA, 20 bp, Artificial Sequence
  Synthetic
agtttcatcttccggcctct

SEQ ID NO: 13 - DNA, 20 bp, Artificial Sequence
  Synthetic
aagcgccactgaatgaaact

SEQ ID NO: 14 - DNA, 20 bp, Artificial Sequence
  Synthetic
aacggcagtcctgtcactct

SEQ ID NO: 15 - DNA, 20 bp, Artificial Sequence
  Synthetic
tgccaatccagcagtcatta

SEQ ID NO: 16 - DNA, 20 bp, Artificial Sequence
  Synthetic
tgaaagcccactctctggtt

SEQ ID NO: 17 - DNA, 24 bp, Artificial Sequence
  Synthetic
cgcagagttatcgtgccagcagat

SEQ ID NO: 18 - DNA, 24 bp, Artificial Sequence
  Synthetic
ccatattctttcaccgcccactcc

SEQ ID NO: 19 - DNA, 20 bp, Artificial Sequence
  Synthetic
atagcagctaccccatggac

SEQ ID NO: 20 - DNA, 20 bp, Artificial Sequence
  Synthetic
tcagacatctggcgttggta

SEQ ID NO: 21 - DNA, 20 bp, Artificial Sequence
  Synthetic
agaatggaaacagcgaagga

SEQ ID NO: 22 - DNA, 24 bp, Artificial Sequence
  Synthetic
tcaatgaaagtaccatcctgatct

SEQ ID NO: 23 - DNA, 20 bp, Artificial Sequence
  Synthetic
gccgtacactgctcaaatca

SEQ ID NO: 24 - DNA, 20 bp, Artificial Sequence
  Synthetic
tttgaagtcatctgggctga

SEQ ID NO: 25 - DNA, 20 bp, Artificial Sequence
  Synthetic
caccggccatacggtggagc

SEQ ID NO: 26 - DNA, 23 bp, Artificial Sequence
  Synthetic
tcgtggtctttggaggcgagagt

SEQ ID NO: 27 - DNA, 22 bp, Artificial Sequence
  Synthetic
tggaggagtggcaatgcctgga

SEQ ID NO: 28 - DNA, 27 bp, Artificial Sequence
  Synthetic
tggcttagtgagagcaatacctgccaa

SEQ ID NO: 29 - DNA, 20 bp, Artificial Sequence
  Synthetic
agccggaccaggcccatctc

SEQ ID NO: 30 - DNA, 20 bp, Artificial Sequence
  Synthetic
cttccgccgcgcatccagaa

SEQ ID NO: 31 - DNA, 20 bp, Artificial Sequence
  Synthetic
ctcctcccgcacggacccat

SEQ ID NO: 32 - DNA, 20 bp, Artificial Sequence
  Synthetic
ttcaagcgctccctgctcgc

SEQ ID NO: 33 - DNA, 24 bp, Artificial Sequence
  Synthetic
gagcacccctatcaaccccctatt

SEQ ID NO: 34 - DNA, 24 bp, Artificial Sequence
  Synthetic
agcaaccctggacctcacacctaa

SEQ ID NO: 35 - DNA, 20 bp, Artificial Sequence
  Synthetic
cgcgagaagatgacccagat

SEQ ID NO: 36 - DNA, 20 bp, Artificial Sequence
  Synthetic
gagtccatcacgatgccagt

SEQ ID NO: 37 - DNA, 21 bp, Artificial Sequence
  Synthetic
tggccacccagagatgcagga

SEQ ID NO: 38 - DNA, 23 bp, Artificial Sequence
  Synthetic
acacactgtccctctttgagcca

SEQ ID NO: 39 - DNA, 20 bp, Artificial Sequence
  Synthetic
tggagctagtgctgcatgtc

SEQ ID NO: 40 - DNA, 20 bp, Artificial Sequence
  Synthetic
ctgccttgctgtgtgaaaaa

SEQ ID NO: 41 - DNA, 20 bp, Artificial Sequence
  Synthetic
ggttcctgggcaggagtaag

SEQ ID NO: 42 - DNA, 20 bp, Artificial Sequence
  Synthetic
aacgtggatcccacactctc

SEQ ID NO: 43 - DNA, 26 bp, Artificial Sequence
  Synthetic
cctagatgaagtctccatgagctaca

SEQ ID NO: 44 - DNA, 21 bp, Artificial Sequence
  Synthetic
gggagggagagctagcacttg

SEQ ID NO: 45 - RNA, 19 nt, Artificial Sequence
  Synthetic
guucuuagcgcacaucuug

SEQ ID NO: 46 - RNA, 19 nt, Artificial Sequence
  Synthetic
ccaauaggcuuaauccugu

SEQ ID NO: 47 - RNA, 19 nt, Artificial Sequence
  Synthetic
gagcaucacuugccuuuaa

SEQ ID NO: 48 - RNA, 19 nt, Artificial Sequence
  Synthetic
agauagagcugcuaaaugu

SEQ ID NO: 49 - protein, 13 aa, Artificial Sequence
  Synthetic
VPQQDWLSQPPAR

SEQ ID NO: 50 - protein, 16 aa, Artificial Sequence
  Synthetic
VIVPADPTLWSTDHVR

SEQ ID NO: 51 - protein, 14 aa, Artificial Sequence
  Synthetic
ETPLPHLTSDDVDK

SEQ ID NO: 52 - protein, 11 aa, Artificial Sequence
  Synthetic
AAQPSPSTVPK

SEQ ID NO: 53 - protein, 9 aa, Artificial Sequence
  Synthetic
MTDPDEVAR

SEQ ID NO: 54 - protein, 17 aa, Artificial Sequence
  Synthetic
YPSDLPYMGSYHAHPQK

SEQ ID NO: 55 - protein, 9 aa, Artificial Sequence
  Synthetic
LPVLAGCLK

SEQ ID NO: 56 - protein, 8 aa, Artificial Sequence
  Synthetic
VFLALAAK

SEQ ID NO: 57 - protein, 11 aa, Artificial Sequence
  Synthetic
VTELALTASDR

SEQ ID NO: 58 - protein, 13 aa, Artificial Sequence
  Synthetic
SLGTIQQCCDAID

SEQ ID NO: 59 - protein, 12 aa, Artificial Sequence
  Synthetic
LNESTFDTQITK

SEQ ID NO: 60 - protein, 17 aa, Artificial Sequence
  Synthetic
INQVFHGSCITEGNELK

SEQ ID NO: 61 - protein, 9 aa, Artificial Sequence
  Synthetic
QDAQVVLYR

SEQ ID NO: 62 - protein, 13 aa, Artificial Sequence
  Synthetic
SQGCSEQVLTVLK

SEQ ID NO: 63 - protein, 11 aa, Artificial Sequence
  Synthetic
DQNILLGTTYR

SEQ ID NO: 64 - protein, 14 aa, Artificial Sequence
  Synthetic
LSDFNDITNMLLLK

SEQ ID NO: 65 - protein, 11 aa, Artificial Sequence
  Synthetic
DSLIFLVDASK

SEQ ID NO: 66 - protein, 17 aa, Artificial Sequence
  Synthetic
IMLFTNEDNPHGNDSAK

SEQ ID NO: 67 - protein, 8 aa, Artificial Sequence
  Synthetic
ILELDQFK

SEQ ID NO: 68 - protein, 12 aa, Artificial Sequence
  Synthetic
DIISIAEDEDLR

SEQ ID NO: 69 - protein, 16 aa, Artificial Sequence
  Synthetic
TFNTSTGGLLLPSDTK

SEQ ID NO: 70 - protein, 14 aa, Artificial Sequence
  Synthetic
SDSFENPVLQQHFR

SEQ ID NO: 71 - protein, 17 aa, Artificial Sequence
  Synthetic
NIPPYFVALVPQEEELD

SEQ ID NO: 72 - protein, 13 aa, Artificial Sequence
  Synthetic
ELVYPPDYNPEGK

SEQ ID NO: 73 - protein, 9 aa, Artificial Sequence
  Synthetic
VEYSEEELK

SEQ ID NO: 74 - protein, 5 aa, Artificial Sequence
  Synthetic
QELLE

SEQ ID NO: 75 - protein, 11 aa, Artificial Sequence
  Synthetic
HIEIFTDLSSR

SEQ ID NO: 76 - protein, 11 aa, Artificial Sequence
  Synthetic
LGGHGPSFPLK

SEQ ID NO: 77 - protein, 10 aa, Artificial Sequence
  Synthetic
LTIGSNLSIR

SEQ ID NO: 78 - protein, 16 aa, Artificial Sequence
  Synthetic
ETVYCLNDDDETEVLK

SEQ ID NO: 79 - protein, 13 aa, Artificial Sequence
  Synthetic
ANPQVGVAFPHIK

SEQ ID NO: 80 - protein, 12 aa, Artificial Sequence
  Synthetic
TDTLEDLFPTTK

SEQ ID NO: 81 - protein, 17 aa, Artificial Sequence
  Synthetic
DQVTAQEIFQDNHEDGP

SEQ ID NO: 82 - protein, 193 aa, Homo sapiens
MIQTVPDPAAHIKEALSVVSEDQSLFECAYGTPHLAKTEMTASSRVPQQDWLSQPPARVTIKMECNPSQVNGSRNSPDECSVAKGGKMVGSPDTVGMNYGSYMEEKHMPPPNMTTNERRVIVPADPTLWSTDHVRQWLEWAVKEYGPHPPALPVTSSSFFAAPNPYWNSPTGGIYPNTRLPTSHMPSHLGTYY

SEQ ID NO: 83 - protein, 13 aa, Artificial Sequence
  Synthetic
MIQTVPDPAAHIK

SEQ ID NO: 84 - protein, 8 aa, Artificial Sequence
  Synthetic
YGTPHLAK

SEQ ID NO: 85 - protein, 16 aa, Artificial Sequence
  Synthetic
TEMTASSSSDYGQTSK

SEQ ID NO: 86 - protein, 13 aa, Artificial Sequence
  Synthetic
VPQQDWLSQPPAR

SEQ ID NO: 87 - protein, 14 aa, Artificial Sequence
  Synthetic
ETPLPHLTSDDVDK

SEQ ID NO: 88 - protein, 14 aa, Artificial Sequence
  Synthetic
SAWTGHGHPTPQSK

SEQ ID NO: 89 - protein, 12 aa, Artificial Sequence
  Synthetic
PQQDWLSQPPAR

SEQ ID NO: 90 - protein, 9 aa, Artificial Sequence
  Synthetic
DWLSQPPAR

SEQ ID NO: 91 - protein, 7 aa, Artificial Sequence
  Synthetic
LSQPPAR

SEQ ID NO: 92 - protein, 6 aa, Artificial Sequence
  Synthetic
SQPPAR

SEQ ID NO: 93 - protein, 5 aa, Artificial Sequence
  Synthetic
QPPAR

SEQ ID NO: 94 - protein, 4 aa, Artificial Sequence
  Synthetic
PPAR

SEQ ID NO: 95 - protein, 81 aa, Homo sapiens
IQLWQFLLELLSDSSNSSCITWEGTNGEFKMTDPDEVARRWGERKSKPNMNYDKLSRALRYYYDKNIMTKVHGKRYAYKFD

SEQ ID NO: 96 - protein, 81 aa, Homo sapiens
IQLWQFLLELLTDKSCQSFISWTGDGWEFKLSDPDEVARRWGKRKNKPKMNYEKLSRGLRYYYDKNIIHKTAGKRYVYRFV

SEQ ID NO: 97 - protein, 84 aa, Homo sapiens
IRLYQFLLDLLRSGDMKDSIWWVDKDKGTFQFSSKHKEALAHRWGIQKGNRKKMTYQKMARALRNYGKTGEVKKVKKKLTYQFS

SEQ ID NO: 98 - protein, 81 aa, Homo sapiens
LQLWQFLVALLDDPSNSHFIAWTGRGMEFKLIEPEEVARRWGIQKNRPAMNYDKLSRSLRYYYEKGIMQKVAGERYVYKFV

SEQ ID NO: 99 - protein, 25 aa, Homo sapiens
LQFLLLIWFARWGKKMYKRLRYKKY